(19) United States
(12) Patent Application Publication
(10) Pub. No.: US 2013/0303941 A1
(43) Pub. Date: Nov. 14, 2013

(54) Method and Apparatus for Evaluating Dynamic Middle Ear Muscle Activity

(71) Applicants: Stephen W. Porges, Chicago, IL (US); Gregory F. Lewis, Chicago, IL (US)

(72) Inventors: Stephen W. Porges, Chicago, IL (US); Gregory F. Lewis, Chicago, IL (US)

(73) Assignee: The Board of Trustees of the University of Illinois, Urbana, IL (US)

(21) Appl. No.: 13/992,450

(22) PCT Filed: Dec. 13, 2011
(86) PCT No.: PCT/US11/64602

§ 371 Date: Jul. 29, 2013

Related U.S. Application Data
(60) Provisional application No. 61/422,296, filed on Dec. 13, 2010.

(51) Int. Cl. A61B 5/12

(52) U.S. Cl. CPC A61B 5/125
USPC 600/559

(57) ABSTRACT

Provided are methods and devices for evaluating dynamic middle ear muscle activity in a subject. A probe is provided having a speaker and a microphone in sound-wave communication with an eardrum associated with the middle ear muscle of the subject. A sound wave is generated from the speaker and transmitted to the eardrum. The sound wave that is reflected is detected and a reflected sound wave property measured. The input sound wave may be comb input to fully extend ossicle movement in all available vibratory modes, thereby providing maximum information as to dynamic middle ear muscle activity.

### CROSS-REFERENCE TO RELATED APPLICATIONS

This application claims the benefit of U.S. Provisional Application No. 61/422,296, filed Dec. 13, 2010, which is specifically incorporated by reference to the extent not inconsistent with the disclosure herein.

### BACKGROUND OF THE INVENTION

Our ability to listen to human voice in background sounds is dependent on the function of our middle ear muscles. The dynamic regulation of the middle ear muscles function as an “anti-masking” mechanism to enable the extraction of human voice from background sounds. As the acoustic features of our modern work and living environments have become more complex and often noisy, our ability to follow verbal instructions is dependent on the adequate functioning of the middle ear muscles. Currently, middle ear muscle function is monitored through clinical tympanometry and acoustic reflex testing, which do not measure the time-varying nature of the middle ear muscles tension. Those clinical tools, although sensitive to severe damage in the neural regulation of the muscles and gross deformations of the bone structure, are not capable of monitoring the dynamic changes in muscle tension necessary to dampen extraneous sounds in the background and to foster the intelligibility of human speech.

### CURRENT STATE OF THE ART

In clinical audiology middle ear function is typically assessed in two ways, tympanometry and acoustic reflex (AR) threshold testing. Tympanometry seals a probe in the auditory canal, applies positive and negative pressure to the outside of the eardrum, and records the volume of the space between the probe and eardrum. Tympanometry can reveal perforations in the eardrum and structural abnormalities in the chain of bones in the middle ear. AR threshold tests measure contraction of the middle ear muscles in response to loud noise. This reflexive contraction is assumed to protect the inner ear by increasing stiffness in the chain of bones. Since the contraction functionally reflects more of the incoming acoustic energy away from the middle and inner ear, AR tests were first based on acoustic immittance and later upon acoustic reflectance measurements. AR tests use either pure tones or broadband noise to elicit the contraction. In testing AR, stimuli are presented at increasing intensity levels until the smallest reliable change in reflected energy necessary is recorded. AR thresholds indicate if and at what intensity the middle ear muscles contract. The existence or lack of a reflex contraction and the intensity of acoustic challenge required to obtain it are relevant clinical features but further parameters of the middle ear muscle function are typically not measured, including resting tension on the middle ear muscles in an active listening environment.

Various devices are disclosed that can be configured for measuring acoustic reflectance, such as U.S. Pat. Nos. 6,048,320, 5,792,072, 3,949,735, 3,757,769, EP Pub. No. 0674874. Those devices and methods, however, do not provide an input sound that is appropriate for assessing the state of a fully activated middle ear. In particular, certain prior art devices are relevant to a static ear in silent environment, as reflected by the prior art that is confined to sound input in the form brief impulses, such as about 100 msec and less.

### SUMMARY OF THE INVENTION

Methods and devices of the present invention provide a rapid, sensitive, reliable and non-abrasive means for evaluating status of the middle ear, including the tension of middle ear muscles. This is relevant as the status of middle ear muscles impact the ability of the middle ear to absorb/reflect sound waves, thereby impacting hearing and sound processing. The middle ear, however, is difficult to characterize in that there are related confounding parameters, including not only the status of the muscles, but the vibration of the interdependent ossicles and also the tympanic membrane. Accordingly, there is a need for methods and devices to better assess status of dynamic middle ear muscle activity, in contrast to methods and devices that assess the status of the static middle ear.

The methods and devices are particularly useful in assessing clinical disorders, including providing information that may be used to determine whether a particular disorder may be relevant for a given individual. Examples include autism, post-traumatic stress disorder, and language delays associated with the processing of human speech in day-to-day environment (e.g., noisy). A substantial fraction of all autistic individuals report auditory hypersensitivities and the underlying mechanism for most is related to the middle ear muscles. Many of the clinical symptoms associated with “central auditory processing” problems are, in fact, due to the “transfer” function of the middle ear structures. If the information (higher harmonics of human speech) is disrupted by the middle ear and not getting to the inner ear, the relevant information for speech processing and language development cannot get to the brain for processing. Accordingly, dynamic middle ear assessment and evaluations is important, making tools that access that assessment and evaluation important and relevant.

Provided herein are various methods, and devices for implementing any of the methods, such as for evaluating dynamic middle ear muscle activity in an ear. Similarly, the method may also be described as measuring a resting tension of middle ear muscles in a subject. Because the method does not rely on subject response, the measure of dynamic middle ear muscle activity and status is objective, fast and reliable, having good repeatability.

In an embodiment, the method is for evaluating dynamic middle ear muscle activity in a subject having ossicles by introducing a non-harmonic acoustic input to an ear of the subject. The non-harmonic acoustic input is specially configured to ensure appropriate movement of the ossicles by use of a comb input that includes frequencies in each of a low frequency range, a middle frequency range and high frequency range. The three frequency ranges span an input frequency range. In an aspect, the frequency range is at least greater than or equal to 100 Hz and less than or equal to 10,000 Hz, such as greater than or equal to 50 Hz and less than or equal to 15,000 Hz, and any sub-ranges therein, as desired. The method is particularly applicable for an ear having an intact ossicle chain having ossicles capable of movement in ossicle directions. In this manner, the non-harmonic acoustic input generates movement of the ossicles in all available ossicle directions. The movement of ossicles in all available ossicle directions by the input is referred to as a middle ear that is “dynamic”. Various other conventional devices in the art, in contrast, suffer from the limitation that the ossicles are not necessarily moving in all possible directions, so that any measurement from those devices and methods cannot be characterized as “dynamic”. The reflected energy from the ear is measured during the non-harmonic acoustic input that generates movement of the ossicles in all available direction, thereby evaluating dynamic middle ear muscle activity. In an aspect, the reflected energy is measured by any of the devices provided herein.

Although the methods provided herein are not limited to any particular frequency range, in one aspect the low frequency range is less than or equal to approximately 1000 Hz; the middle frequency range is greater than approximately 1000 Hz and less than approximately 3000 Hz; and the high frequency range is greater than or equal to approximately 3000 Hz.

In an embodiment, the measuring step has a measuring time period and the non-harmonic acoustic input is continuously introduced to the ear during the measuring time period. In an aspect, the time period is about 0.5 seconds, about 1 second, about 10 seconds, or is greater than and equal to 0.5 seconds and less than or equal to 10 seconds.

In an aspect, the non-harmonic acoustic input is continuously introduced to the ear for a time that is greater than or equal to 0.5 second and, optionally, less than or equal to 20 seconds.

In an embodiment, the reflected energy is measured over a measuring frequency range and dynamic middle ear muscle activity is obtained as a function frequency. The measured reflected energy, such as a magnitude, is optionally displayed or otherwise quantified and communicated to the subject or the researcher. In an aspect, the measuring frequency range is selected from a range that is greater than or equal to 200 Hz and less than or equal to 5000 Hz.

In an embodiment, the evaluating is by obtaining a magnitude of the reflected energy at a measured frequency. In an embodiment, the evaluating is by obtaining a phase shift of the reflected energy at a measured frequency. In an aspect, the method further comprises comparing the obtained magnitude against a reference from a normal subject, or from a population of normal subjects. In this manner, the magnitude of the reflected energy over a range of measured frequency can be compared to a reference.

The method can be used with any number or variety of algorithms useful in comparing values or data plots. For example, the most straightforward algorithm is calculating a difference between the obtained magnitude and the reference magnitude at a one or more measured frequency that is within the range of measured frequency. More complex and/or fine-tuned algorithms may be used to more precisely detect differences between a subject and reference, such as by weighting values at a certain frequency, frequencies, or ranges to provide greater emphasis to the differences at certain frequencies. Accordingly, a composite measure may be calculated by weighting at a one or more weighted frequency value. In an aspect, the weighted frequency value corresponds to a frequency associated with an atypical hearing condition or a sound processing defect. This aspect recognizes that, depending on the atypical condition, certain frequencies may be more relevant than others. Similarly, depending on the subject, certain frequencies may be more relevant (e.g., young versus old).

In an embodiment, any of the methods further relate to using an algorithm to provide quantification of the reflected or absorbed energy in terms of typical/atypical, normal/abnormal or pass/fail, for one or more conditions. Other parameters besides a weighted frequency may be used to provide more tailored or specific information. For example, areas or shapes defined by the curve over a frequency range. One useful portion of the curve is the profile in the region of the higher formants of speech, such as about 1200-3500 Hz. The width and depth of a bowl or cup region of the plotted data can be used to provide statistical information useful in providing information as to whether a subject is atypical, such as a description of the cup width (e.g., inflection point position), depth of the cup, curvature or slope at particular frequencies, etc.

In an aspect, the atypical hearing defect is difficulty in hearing speech in a noisy environment and the weighted frequency value is selected from a frequency that is greater than 1300 HZ; hypersensitivity to speech and the weighted frequency value is selected from a frequency that is between about 1300 Hz and 4000 Hz; hearing loss and the weighted frequency value is selected from a frequency that is between about 1000 Hz and 5000 Hz; hypersensitivity to noise and the weighted frequency value is between about 50 Hz and 1000 Hz; or impaired language development and the weighted frequency value is greater than 1300 Hz. Other parameters useful may be used by an algorithm. For example, an area under or between curves may be calculated. The curvature, profile depth and/or profile width may be quantified and used to assist in quantifying the difference between the subject and reference.

In an embodiment, the comb input comprises a plurality of components each having a non-harmonic frequency, said components spanning a frequency range that is greater than or equal to about 50 Hz and less than or equal to about 15000 Hz. In this manner, the comb input spans the vibration modes of the ossicles. In an aspect, at least two components are provided in each of the low, middle and high frequency ranges. In an aspect, the components have a total number selected from a range that is greater than or equal to 3 and less than or equal to 100. In an aspect, the component number is greater than or equal to 10 and less than or equal to 20. In an aspect, the component number is 15. In an aspect, the comb input comprises components that are not integer harmonics.

In an aspect, the components have substantially equivalent power levels to the other components, and said power levels remain substantially constant during said introducing step. In an aspect, the components each have the same power level.

In an aspect, the power or amplitude of the components is selected to be sub-threshold or substantially sub-threshold, so that an acoustic reflex response of the middle ear muscles is avoided.

In an embodiment, any of the methods provided herein further comprise selecting the comb input to minimize or avoid generating standing waves of air pressure on the reflected energy. In this manner, harmonic components with respect to the ear canal are avoided. In addition, integer harmonics within the comb input are avoided (e.g., no component is an integer multiple of another component).

In an aspect, each component is a non-square wave having a full-width at half-maximum that is less than or equal to 10 Hz, less than or equal to 5 Hz, or less than or equal to 1 Hz.

Any of the methods provided herein optionally relate to an evaluating step that is determining the difference between the measured reflected energy and a normal reflected energy from a normal subject. In an embodiment, the middle ear muscle activity is identified as atypical.

Any of the methods related to obtaining information useful for diagnosing a middle-ear related abnormality, wherein the abnormality is selected from the group consisting of: conductive hearing loss; auditory processing deficits; noise hypersensitivity; speech hypersensitivity and speech hyposensitivity. In an aspect, the information corresponds to higher reflected energy at a higher frequency, wherein the higher frequency is greater than or equal to 1000 Hz, 1200 Hz, 2000 Hz, or is between 1200 Hz and 4500 Hz.

In an embodiment, the method further comprises quantifying dynamic middle ear muscle activity for a subject suspected of a clinical disorder or under a therapeutic treatment of a clinical disorder. In an aspect, the clinical disorder is autism, post-traumatic stress disorder, language delay, language disorder, or hearing disorder.

Any of the methods provided herein may be performed on the left ear, the right ear, or both left and right ear, such as simultaneously or separately and sequentially. In an aspect, the method further comprises presenting a middle ear muscle acoustic challenge to an ear contralateral to the ear in sound-wave communication with the non-harmonic acoustic input.

The methods and devices provided herein can be useful in assessing the effectiveness of a therapeutic intervention, such as by the subject with a therapeutic intervention and monitoring the effectiveness of the therapeutic intervention by repeating the evaluation of dynamic middle ear activity after the therapeutic intervention.

In an aspect, any of the methods provided herein further comprise introducing a probe tone to the ear at a frequency and intensity selected to minimize variation in the reflected energy across different subjects.

In an embodiment, any of the methods disclosed herein may be described as measuring a resting tension of middle ear muscles in a subject having an intact ossicle chain by exciting each ossicle of the ossicle chain by introducing a non-harmonic acoustic input to an ear of the subject, thereby causing each of the ossicles to move in all available ossicle movement directions. In other words, the input frequencies are selected so that the ossicles vibrate in all modes, thereby fully extending the ossicles in each mode (range of motion). Reflected energy from the ear during the non-harmonic acoustic input that generates movement of the ossicles in all available directions is measured, thereby measuring the resting tension of middle ear muscles. In an aspect, the measured resting tension of the middle ear muscle provides information useful in diagnosing a hearing or psychiatric condition. In an aspect, the acoustic input is sub-threshold or substantially sub-threshold. In an aspect, the acoustic input, or a portion thereof, is at or above threshold, so that the subject undergoes an acoustic reflex, and the device or method provides information related to muscle activity before, during and/or after the acoustic response. In one embodiment, overlaying the comb input is a probe input of a selected frequency and intensity sufficient to elicit an acoustic reflex response.

In an embodiment, any of the methods described herein provide a high-reliability status of the middle ears of both ears of the subject is assessed in an assessment time that is fast, such as less than or equal to five minutes. In an embodiment, the method is characterized as non-intrusive or non-abrasive, in that the need for chirping, clicking or other audible sounds is not necessary.

In another embodiment, provided is a device for measuring a resting tension of middle ear muscles in an active ear of a subject. In an aspect, the device comprises a signal generator for generating a steady-state non-harmonic acoustic input comprising a comb input; a speaker for emitting a sound wave that is generated from the signal generator; a probe containing the speaker for positioning the speaker in sound-communication with an ear. The emitted sound wave vibrates ossicles of an intact ossicle chain of the ear in all available ossicle directions. A microphone is in sound wave communication with the speaker for detecting a reflected sound wave of the emitted sound wave during ossicle vibration in all available ossicle directions and a processor for calculating changes in an acoustic transfer function from middle ear muscle movement based on a reflectance phase shift or magnitude change between the emitted sound wave and the reflected sound wave. The emitted sound wave, detected reflected sound wave, and calculated acoustic transfer function are continuous and synchronized with the emitted sound wave.

In an aspect, the acoustic transfer function is calculated by spectral analysis with frequency dependent resolution having a tolerance for each component of the comb signal within 0.1 radians per second, thereby minimizing effects of bodily noise.

In an embodiment, the comb input comprises a plurality of components each having a non-harmonic frequency, the components spanning a frequency range that is greater than or equal to about 50 Hz and less than or equal to about 15000 Hz, and at least one component is in each of a low frequency range that is less than or equal to about 1000 Hz, a middle frequency range greater than approximately 1000 Hz and less than approximately 3000 Hz; and high frequency range greater than or equal to approximately 3000 Hz.

We describe a new assessment method and apparatus to dynamically evaluate the temporal features of middle ear muscle function. Middle ear muscle function is thoroughly characterized by monitoring the acoustic transmission properties of the measured ear, the acoustic transfer function (ATF). As used herein, “ATF” refers to the formula which relates incoming sound energy, measured at the eardrum, to perceived sound energy, as exists within the sense organ of the cochlea. The ATF encompasses the two parameters of this frequency dependent formula, magnitude and phase. Acoustic energy reflectance at the eardrum is inversely related to the ATF. The Reflectance Transfer Function (RTF) relates incoming sound energy within the ear canal to outgoing sound energy at the same position in the ear canal. As used herein “reflectance properties” refers to components of the total RTF. Contraction of the middle ear muscles alters the ATF and the RTF. The method and device estimate a subject's ATF from a baseline measure of the RTF, the energy reflectance properties at one or more frequencies. The method and device quantify changes in the RTF in both the time and frequency domain. The technology has applications in clinical audiometry as well as in the identification of potential mechanisms underlying or contributing to several clinical features including hyperacusis, central auditory processing difficulties, and difficulties in listening to speech in noisy environments.

The methods and devices described herein facilitate the extraction of new information describing middle ear muscle function that is not attainable through either tympanometry or AR threshold testing. A new method is described for tracking changes in the energy reflectance properties of the tympanic membrane. Middle ear muscle contraction alters the ATF and also these reflectance properties. Due to individual differences in physical structure and neural regulation, the functional impact of muscle contractions varies widely between individuals. Within individuals, middle ear muscle function is variable as muscle tone varies from flaccid to contraction. The new technology provides an opportunity to assess both supra- and sub-reflexive levels of contractions and to measure changes in middle ear muscle status in response to various acoustic challenges (e.g., words in noise, music, etc.), as well as psychological state (e.g., anxiety, focus, etc.). Thus, the method provides the first demonstration of dynamic adjustments of the middle ear muscles at and below the threshold required to elicit the AR, and the capacity to measure, assess and make diagnosis based on one or more measured parameters related to middle ear muscle activity including a reflected sound wave phase shift and reflected sound wave change in intensity or magnitude at one or more carrier frequencies within a probe tone.

Provided herein are methods and devices for evaluating dynamic middle ear muscle activity. The methods and devices provide increased sensitivity, including evaluations in the sub-threshold stimulus range and expanded temporal resolution. Conventional methods, in contrast, provide evaluation related to a response at a measured threshold stimulus that elicits an AR. In an aspect, the invention measures a property of a sound wave that is generated from the probe, and subsequently reflected off the eardrum, such as by measuring the reflected sound wave energy (e.g., intensity or magnitude) or by the phase shift of the reflected sound wave. In an aspect a plurality of pure tones are combined in the probe sound wave, and the phase and magnitude of each component of the reflected wave is tracked. In an aspect the individual phase and magnitude signals are combined to create a more sensitive global measure of middle ear muscle function. Movement of the middle ear impacts the movement or properties of the eardrum, which in turn will affect the reflected sound wave property. The reflected wave property is used to characterize or evaluate middle ear movement. In an aspect, the information used for diagnosis relates to reflected energy from the active (or dynamic) middle ear, during the comb input, including comb input that is sub-acoustic or partially sub-acoustic.

Middle ear movement characterization or evaluation is useful to provide diagnosis of a patient's hearing or to diagnose a hearing condition, such as a hearing condition requiring additional testing, intervention or treatment. In an aspect, the device and method relates to a generated sound wave that is sub-threshold in intensity. “Sub-threshold” refers to an intensity that is less than the intensity required to elicit an acoustic reflex related to tetanic contraction. In an aspect the stimulus is ipsilateral. In an aspect the stimulus is contralateral. In an aspect the stimulus is both ipsilateral and contralateral. In an aspect, the sound wave generated by the probe (and in which the probe detects a corresponding sound wave reflected from the ear) is a sine wave or a more complex sound wave such as that corresponding to the combination of multiple sine waves. One or more parameters of the reflected sound wave can be used to characterize the dynamic response of middle ear activity, including a characterization that indicates the presence, absence, or deficiency of middle ear muscle activity. In this manner, the devices and methods are capable of assessing activity for generated sound waves at intensities that corresponding AR and tympanometry devices cannot assess.

### BRIEF DESCRIPTION OF THE DRAWINGS

FIG. 1 is a flow diagram of one embodiment of the method and device.

FIG. 2: Spectrogram of text-to-speech recording of the number eight. Note the spectral density in the frequency region 1200 to 4500 Hz.

FIG. 3: Spectrogram of text-to-speech recording of the number four. Note the spectral density in the frequency region 1200 to 4500 Hz.

FIG. 4: Spectrogram of text-to-speech recording of the number seven. Note the spectral density in the frequency region 1200 to 4500 Hz.

FIG. 5: Spectrogram of the noise component of the numbers in noise task. This masking noise was combined with the text-to-speech recordings (see FIGS. 2-4). Note the restriction of energy to frequencies below 1000 Hz.

FIG. 6: Recorded noise levels from one trial of the Numbers in Noise task. The solid line indicates the noise level at the end of a run of correct responses and the dashed line the level at the end of a run of incorrect responses. The noise level is linearly related to dB SPL and the units are arbitrary. The box indicates the final responses used to calculate the 50% threshold for detection.

FIG. 7: Spectral density of the MESA stimulus signal. Note the equal intensity of the narrowband components in the signal.

FIG. 8: Block diagram of the MESA measurement setup. The circle represents the subject's ear canal, within which the probe is placed.

FIG. 9: EqL measurement in the right and left ears. Note the similarity between the psychoacoustic measures in each ear.

FIG. 10: Three right ear measurements from one subject. The dashed line represents a normative measure, based on a small sample collected during pilot testing of the device. The probe was replaced between the second and third recordings.

FIG. 11: Two left ear measurements from one subject. The dashed line represents a normative measure, based on a small sample collected during pilot testing of the device. The probe was replaced once between the recordings.

FIG. 12: Between-subject variance in MESA at each frequency. Note the minima around 1000 Hz, the point of normalization for the measure.

FIG. 13: MESA measurements in each ear. Error bars represent +/−1 SE.

FIG. 14: Correlation between MESA at each frequency and NiN_{—}50 in the right ear. N=17, * indicates p<0.05. Note the negative correlations across the range of frequencies above 1000 Hz.

FIG. 15: Correlation between MESA at each frequency and NiN_{—}50 in the left ear. N=17, * indicates p<0.05. Note the significant correlations at the lowest frequencies measured.

FIG. 16: Scatter plot: right ear noise tolerance and MESA mid-frequency level. Note the strong correlation between the summary statistic and NiN_{—}50. This indicates that subjects with the greatest absorption of energy in the mid-frequency range tolerated the highest levels of noise in the speech intelligibility task.

FIG. 17: Scatter plot: left ear noise tolerance and MESA low-frequency level. Note the correlation between the summary statistic and NiN_{—}50. This indicates that subjects with the greatest reflection of energy in the low-frequency range tolerated the highest levels of noise in the speech intelligibility task.

FIG. 18: Right Ear MESA Profile: Split-half groups based on NiN-R. Error bars represent +/−1 SE of the mean (high noise tolerance group, N=8; low noise tolerance group, N=9).

FIG. 19: Left Ear MESA Profile: Split-half groups based on NiN-L. Error bars represent +/−1 SE of the mean (high noise tolerance group, N=8; low noise tolerance group, N=9).

FIG. 20: Right Ear MESA below 1000 Hz. Error bars represent +/−1 SE of the mean (high noise tolerance group, N=8; low noise tolerance group, N=9).

FIG. 21: Left Ear MESA below 1000 Hz. Error bars represent +/−1 SE of the mean (high noise tolerance group, N=8; low noise tolerance group, N=9).

FIG. 22: Correlation between left ear loudness scaling and individual frequencies of the MESA measure. Note the consistent pattern of positive correlations for the frequencies greater than 1000 Hz. N=17. +p<0.01, *p<0.05.

FIG. 23: Scatter plot of MESA and EqL scaling in the left ear. Note the strong correlation, r(17)=0.77, p<0.001. No such relationship existed for the right ear measures.

FIG. 24: Mean MESA for individuals with large and small loudness scaling in the left ear. Individuals with flatter profiles on the equal loudness task had less advantage for absorption above 2000 Hz. Error bars represent +/−1 SE (Large difference, N=8, Small difference, N=9).

FIG. 25: Correlation between the composite hyperacusis score C and each frequency of MESA. Note the strong left ear correlations with the lowest frequencies, the directionality change around the normalization point of 1000 Hz, and the similar relationship above 1000 Hz for both the right and left ears. * indicates p<0.05.

FIG. 26: Subject's right ear MESA profile at pre and post testing. Note the consistent measurement with a new probe used at each session.

FIG. 27: Subject's left ear MESA profile at pre and post testing. Note the change, with a region of increased absorption that is both wider and generally deeper above 2000 Hz

FIG. 28: Left ear MESA profile after one week of the auditory intervention and during pretesting at the two-month follow-up.

FIG. 29: Right ear MESA profile at pre and post testing during the follow-up visit. Note again the lack of change in the subject's right ear measurement.

FIG. 30: Right ear MESA profile at pre and post testing during the follow-up visit. Note again the change in the same direction as during the initial auditory intervention.

FIG. 31: Summary of left ear MESA measures for this case study. Note the consistent change in the left ear at the initial intervention and following only 75 minutes of audio at the follow-up visit. At post-testing the subject had a greater advantage for absorbing the frequencies of the higher formants than the normal hearing subjects.

FIG. 32: Frequency spectrum of reflected energy (relative to 1000 Hz) obtained from middle ear sound absorption system (MESA) from the left year of a normal and a test subject.

FIG. 33: Frequency spectrum of reflected energy (relative to 1000 Hz) obtained from middle ear sound absorption system (MESA) from a normal subject and a test subject with a reported hypersensitivity to speech sound.

### DETAILED DESCRIPTION OF THE INVENTION

The invention may be further understood by the following non-limiting examples. All references cited herein are hereby incorporated by reference to the extent not inconsistent with the disclosure herewith. Although the description herein contains many specificities, these should not be construed as limiting the scope of the invention but as merely providing illustrations of some of the presently preferred embodiments of the invention. For example, the scope of the invention should be determined by the appended claims and their equivalents, rather than by the examples given.

“Middle ear” refers to the portion of the ear internal to the eardrum and external to the oval window of the cochlea. In particular, the middle ear has three ossicles that vibrate, thereby transducing sound wave energy in air to a form that can be processed downstream in the ear (e.g., fluid waves in the cochlea). The middle ear also contains muscles that influence the movement of ossicles. The muscles may contract in response to loud sounds, effectively reducing the impact of loud sounds on the inner ear. This is referred to as the acoustic or tympanic reflex. Typically, the muscles have some resting tension, wherein the resting tension can vary between subjects.

“Middle ear muscle activity” refers to the action of the middle ear muscles on the acoustics in the ear, such as the amount of energy absorbed/reflected at the inner ear or middle ear.

“Dynamic middle ear muscle activity” refers to an evaluation of energy reflection/absorption while the ossicles are fully vibrating in each of the modes (see, e.g., Koike et al. J. Acoust. Soc. Am. 111(3):1306-1317 (2002)), so that there is movement in all possible directions, and generally with a maximum range of motion. The dynamic middle ear muscle activity may, however, occur for middle ear muscle that is at rest, under tension, or partial tension. In an aspect, the methods and devices provided herein are used for middle ear muscle that is at rest.

“Non-harmonic acoustic input” refers to a sound wave that is selected to span the frequency range of the ossicle modes but that minimize the build-up of standing waves of air pressure on the reflected energy. In this manner, the input fully extends the ossicles in each range of ossicle motion (i.e., mode) and, therefore, the reflected energy conveys the maximum amount of information on the resting tension of the middle ear muscles. Optionally, the input further comprises a probe signal component, including an adjustable probe signal in terms of frequency and amplitude. Optionally, the probe signal is sufficiently loud to elicit an acoustic response contraction and the comb input is used to observe middle ear muscles return to a “listening” state after the acoustic response contraction relaxes. In an aspect, the non-harmonic acoustic input of any of the methods provided herein is at a level that is sub-threshold, or significantly sub-threshold.

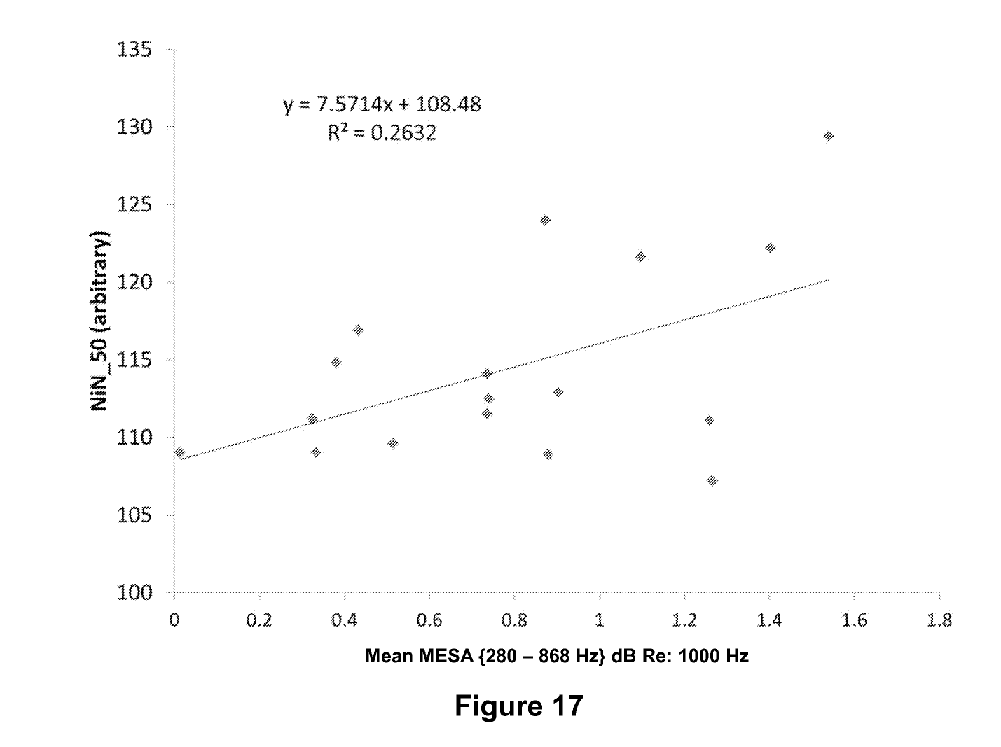

“Comb input” refers to the portion of the non-harmonic acoustic input that are individual components at individual frequencies, with each component having a narrow frequency spread and equivalent power to other components (see, e.g., FIG. 17). Accordingly, in this aspect “components” refer to the individual spike within the comb input. “Non-square wave” refers to a leading and/or lagging edge of a pulse that is not vertical. In addition, a non-square wave can have a well-defined full-width at half maximum. In contrast, a square-wave has leading and lagging edges that are vertical, and the width of the wave is generally independent of the fraction of maximum. In an embodiment, the non-square wave component has a slope that is within 10%, 5% or 1% of the slopes illustrated in FIG. 17.

A component that is not an “integer harmonic” refers to the frequency of a component that is not a multiple of any other component frequency in the comb input, thereby improving sensitivity and decreasing unwanted distortion.

“Reflected energy” refers to the input sound energy that is reflected from the ear and detected by a sensor. Reflected and absorbed energy equal the energy introduced to the ear in the form of an acoustic input. Knowing one parameter, therefore, provides the ability to calculate the other as the energy input is a known variable. Accordingly, higher reflected energy values can be associated with hearing loss, as there is less energy available to generate hearing-related signals to, for example, the brain for processing. A “transfer function” is the operator that relates the input energy to the reflected energy, and the transfer function can change depending on MEM activity or state.

As used herein, a power level is “substantially equivalent” if the difference in power between individual components is less than about 10%, less than about 5%, or less than about 1%.

“Atypical” refers to a measured reflected energy (or calculated absorbance) that is statistically significantly different from a reference or a normal individual.

A “reference” refers to a dynamic middle ear muscle activity from one or more persons that do not suffer abnormal hearing or sound processing. The reference may be from a library of such persons, so that statistical parameters are provided over a frequency range, such as an average, standard deviation, or other measure of confidence level. In this manner, evaluation of a subject can be better quantified with respect to confidence level that the dynamic response is statistically significantly different from normal, such as falling outside a predetermined number of standard deviations, or at a 95% or greater confidence level. Similarly, as desired and depending on the frequency range of interest, particular frequencies may be “weighted” to provide improved statistical analysis for determining whether an individual's measured reflected energy is typical or atypical. In this manner, important frequency values or ranges can be afforded more weight, so that differences between measured and reference are emphasized compared to other differences that may be less pertinent for the particular atypical hearing defect. The reference or normal may be obtained from the device itself or may be from a library of data.

As used herein, “significantly sub-threshold” refers to a sound intensity that is less than half the intensity required to recruit the brainstem acoustic reflex. In an aspect, the comb input is provided to the subject at an intensity that is sufficiently low so that there is no acoustic reflex response by the subject.

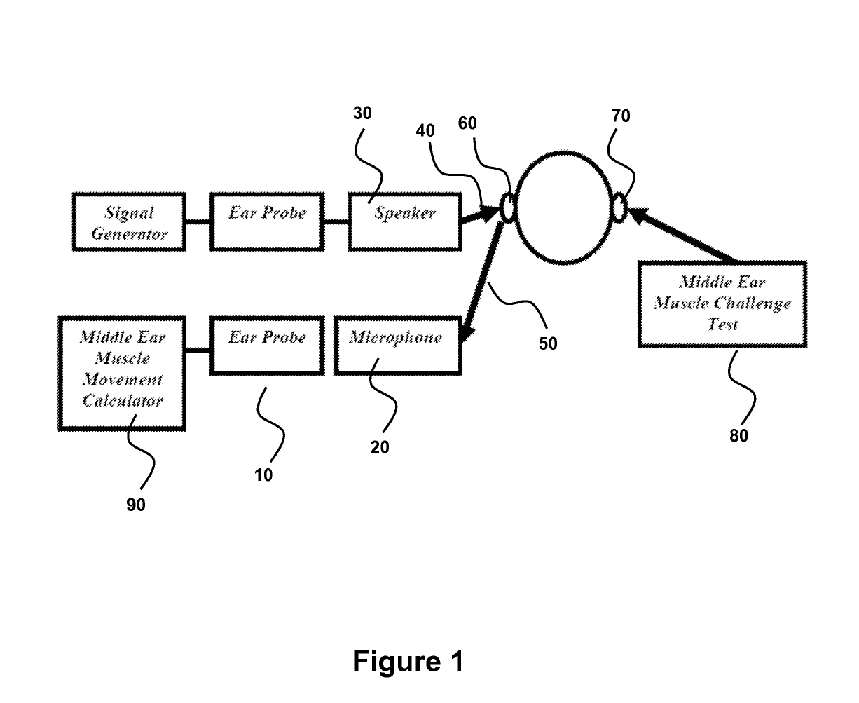

FIG. 1 provides a flow diagram of the device. In one ear a probe 10 is placed that contains both a small microphone 20 and a small speaker 30. Within the signal generator, a series of sinusoidal signals are combined by a digital processor to create the probe tone 40. This digital signal, Dᵢₙ, is converted to an analog voltage and driven through the speaker 30 located in the Ear Probe 10, creating a pressure wave in the ear canal that reflects off the measurement ear 60. The reflected wave 50 is converted to an analog voltage signal within the Microphone 20. The reflected wave 50 is digitized to create Dₒᵤₜ, a time synchronous representation of the reflected probe signal. In an aspect the reflected wave is filtered before digitization. In an aspect the digitized reflected wave is filtered before transfer function estimation.

The movement calculator 90 receives the two digital signals in bins of a fixed number of samples Nₛ. Thus, for a single calculation Dᵢₙ(1 . . . Nₛ) and Dₒᵤₜ(1 . . . Ns) are used to estimate the RTF based upon changes in the properties of the reflected wave. In an aspect, the output from the device is an intensity of the reflected sound wave, such as an intensity at a frequency, wherein the intensity is measure over a range of frequencies.

In an aspect, the RTF is estimated through spectral analysis consisting of Discrete Fourier Transformation of the input and output. In an aspect, the RTF is estimated through spectral analysis consisting of autoregressive modeling of the input and output. In an aspect, the RTF is estimated through spectral analysis consisting of Discrete Wavelet Transformation of the input and output.

Continuous output of the RTF estimate is generated by the processor. In an aspect, time varying amplitude of the carrier frequencies are visualized in real-time. In an aspect, time varying phase of the carrier frequencies are visualized in real-time.

In an aspect, properties of the multiple sinusoidal components are combined to create an optimal, individualized measure of middle ear muscle contraction.

In an aspect, the RTF in the absence of acoustic challenge is used to estimate the baseline acoustic transfer function, ATF. In an aspect, changes in reflectance properties during acoustic challenge are combined with the baseline ATF estimate to calculate dynamic changes in the ATF.

In an aspect, the baseline RTF is used to estimate the ATF, which is stored in memory. This ATF is combined with time-varying reflectance properties to allow real time visualization of the changing ATF.

As the middle ear muscles contract, the ossicles and eardrum are displaced, changing the distance between the source signal and the point of reflection. These changes in reflectance location within the middle ear alter the phase and magnitude of the probe in a frequency specific manner. By monitoring acoustic reflectance properties continuously at a plurality of frequencies, it is possible to detect middle ear muscle activity in response to acoustic stimulation below the threshold level of the acoustic reflex. By synchronizing the presentation of the acoustic challenge and the recorded signal, time parameters of the muscle response are calculated. By presenting middle ear muscle acoustic challenges 80 in the contralateral ear 70, the method facilitates assessment of dynamic middle ear muscle adjustments to shifts in signal and noise levels, as well as the signal to noise ratio (i.e., voice embedded in background sounds). This information has previously not been available. Examples of various components useful in the devices and methods disclosed herein are provided in the art, such as EP 0674874, U.S. Pat. No. 3,949,735 and PCT Pub. No. 2006/101935.

### Example 1

### Assessment of Resting Middle Ear Muscle Tone by a New Measure of Energy Reflectance

### List of Abbreviations in Example 1

### ASR—Acoustic stapedial reflex; dB—ecibel; dB(A) and dB(C)—Weighting scales for noise exposure; DPOAE—Distortion product otoacoustic emissions; MESAS—middle ear sound absorption system; MESA—Middle Ear Sound Absorption; EqL—Equal loudness contour measured by custom software; GUI—Graphical user interface; Hz—Hertz (1/second); LE—Left Ear; ME—Middle ear; MEM—Middle ear muscle; NiN—Numbers in Noise; NiN_{—}50—Output of NiN test, the 50% threshold for detection; OAE—Otoacoustic emissions; RE—Right Ear; SPL—Sound pressure level (dB Re: 20 μPa)

This example investigates the covariation between neural regulation of the middle ear muscles and functional measures of hearing associated with sensitivity to noise and the ability to understand spoken words in the presence of noise. The example employs a novel measure of sound reflection and absorption within the ear canal. Study design includes measurement parameters designed to test a model linking the neural regulation of the autonomic nervous system to the neural regulation of the striated muscles of the face and head.

MESAS measurements from the new device are contrasted with a new psychoacoustic measurement of hearing-in-noise performance, a standard psychoacoustic measurement of loudness scaling, and two self-report measures of hearing sensitivity. Middle ear muscle tone varies as a function of individual differences in neural regulation of peripheral sensory gating structures. MESAS measurements are optimized to maximize individual differences in energy reflectance from the ear canal due to variance in resting middle ear muscle tone.

Significant lateral differences in the functional impact of MESAS related to loudness perception and speech intelligibility are identified. In the right ear, the relationship between increased absorption of frequencies corresponding to the higher formants and improved speech intelligibility is confirmed. In the left ear, the hypothesized relationship existed between increased loudness differences between high and low tones and energy absorption in the frequency range of the higher formants is confirmed. These findings have implications for the autonomic regulation of peripheral sensory gating structures during a quiescent state. Differences in neural regulation may be lateralized in healthy populations, with functional significance in hearing and listening. Greater differences may occur in clinical populations, particularly those with receptive language difficulties. The measurements developed in this example improve the ability to measure these parameters of health and disease and to develop treatments that improve language reception.

This example investigates the covariation between neural regulation of the middle ear muscles and functional measures of hearing associated with sensitivity to noise and the ability to understand spoken words in the presence of noise. This effort is based on a theoretical model linking the neural regulation of the autonomic nervous system to the neural regulation of the striated muscles of the face and head as an integrated social engagement system to facilitate socially appropriate behaviors (Porges & Lewis, 2010). A social engagement system characterized by the integrated regulation of visceromotor (e.g., heart, lungs, etc.) and somatomotor components is unique to mammals (see Porges, 2007). The middle ear muscles are a component of this social engagement system.

The mammalian middle ear is a highly specialized transducer that couples the atmospheric environment to the inner ear sensory system. An understanding of the mechanisms and functions of the middle ear (ME) system has increased as technologies have improved and have enabled more sensitive measurements. The ME is a mechanical transducer, transforming airborne pressure waves into fluid borne waves within the cochlea. The ME is one in a series of filters along the transmission pathway from the environment to the brain-dependent processes resulting in the perception of sound. Transfer functions determine the mathematical relationship between the input and output from a system (i.e., the gain and delay as a function of frequency). Each filter in the auditory system has a transfer function and the estimation of these functions provide a better understanding of how the subjective perception of sound is related to the distribution of acoustic energy in the environment.

The first filter encountered by acoustic pressure waves is the external ear (pinna), followed by the auditory canal, the middle ear, the cochlea, and neural filters within the central auditory system. We measure small changes in the transfer function of the middle ear and energy reflection in the sealed ear canal. The ear canal filter is included in the measurement obtained, although preferably we measure variance in tension applied by two small muscles in the middle ear. The ear canal resonance (i.e., peak in the gain of the transfer function) has a peak around 2000 to 3000 Hz. It is assumed that the magnitude of this peak is partly a function of the status of the tympanic membrane (the ear drum). The tympanic membrane is the outermost aspect of the middle ear. Since changes in muscle tension within the middle ear change the characteristics of the tympanic membrane, and the ear canal system, the behavior of the whole system is considered.

The tympanic membrane is attached to the first of three small bones (ossicles) that transfer acoustic pressure wave into the fluid of the cochlea. The first bone in the ossicle chain is the malleus. The malleus is attached to the first of two muscles in the ME, the tensor tympani. As the malleus vibrates in response to acoustic pressure changes (i.e., waves) at the tympanic membrane, it induces motion in the second ossicle (the incus), which is coupled to the final ossicle (the stapes). Several aspects of this power transformation highlight the dependence of the auditory system on the ME.

Sensory systems transduce environmental information into neural impulses that are decoded and interpreted by central cortical networks. Sensory systems (e.g., vision, hearing, tactile, etc.) share the essential feature that they compress the range of environmental signals into a manageable range of biological values (e.g., single neuron firing rates). The ME plays an essential role in compression within the auditory system and functions similar to an automatic gain control that enables a more linear processing within a restricted range by higher neural circuits (Zwislocki, 2002). The acoustic stapedial reflex (ASR) is an example of an aspect of this automatic gain control. Loud sounds, detected in the cochlea, trigger a bilateral brainstem reflex that contracts the stapedius muscle, reducing the transmission of acoustic energy into the cochlea. The attenuation of acoustic energy transmission to the cochlea mediated by the ASR is frequency dependent (Pang, 1997; Liberman, 1998). Greater attenuation occurs to frequencies below 1000 Hz than to those above 1000 Hz (Pang, 1989). This transition point, from the maximum attenuation provided below 1000 Hz to the progressively smaller attenuation above 1000 Hz, coincides with the maximal gain provided by the ME structures (in an extracted preparation that does not include muscles, tendons or neural input). This gain maxima, between 1000 and 1200 Hz (Aibara, 2001), is the resonant frequency of the middle ear (in the absence of any soft tissue components). The 1000 Hz reference point for a roll-off in ME transmission, as a function of tension on the stapedial muscle has also been demonstrated in electroacoustic models of the middle ear (Lutman, 1979).

This example provides a new technology to identify and describe, in addition to this large transitory compression of low-frequency acoustic energy during reflexive contraction, a more tonic individual difference in the magnitude of resting middle ear muscle tension. The features of muscle tone influence the filter characteristics, although the features are characterized by individual differences of a magnitude noticeably smaller than the reflex.

Middle ear (ME) structures filter features of the acoustic environment and limit the transmission of acoustic energy to the inner ear and the central nervous system. Within the disciplines of speech and hearing sciences, the filter characteristics of ME have been minimally investigated. In contrast, these disciplines have placed a greater emphasis on the “downstream” structures (e.g., inner ear) and neural circuits (e.g., brainstem and cortical event related potentials) that are involved in processing acoustic information related to speech perception and language development. Current approaches to the study of ME structures have focused on the reflexive nature of the stapedius muscle (i.e., acoustic reflex). Additionally, ME structures have been evaluated to determine the physical nature of the ME. Clinically, this technique has been used to identify pathological conditions including ossification of the ME bones and tympanic membrane perfusion (Allen, 2005) and otitis media with effusion (Beers, 2010). Little attention has been paid to individual and situational differences in middle ear muscle (MEM) tone, which may bias or distort the acoustic information being processed via these downstream structures (e.g., olivary cochlear filtering).

This example tests the hypothesis that individual differences in MEM tone influences loudness, speech intelligibility, and the self-perception of noise sensitivity (i.e., hyperacusis). Loudness is a perceived construct, it only exists for the individual hearing the sound in that loudness is a judgment of intensity not an objective measurement. Loudness is the sense of intensity from barely detectable through the audible range up to intensities that cause physical pain. Speech intelligibility is defined for the purposes of this study as the functional ability to identify a spoken word by selecting it from a set of possible words. Human communication is complex and communication is possible without perfect word recognition. A simple measure of intelligibility is selected to minimize the contribution of cognitive factors such as attention and memory to the speech intelligibility index.

Hypersensitivity: Heightened sensitivity to sound is a feature of several psychiatric disorders (e.g., Williams syndrome, autism spectrum disorders (ASD), schizophrenia) (Khalfa, 2004). Conflicting reports (Gordon, 1986; Katzenell, 2001) have proposed a link between ME function and hypersensitivity to sound, although most admit that the disorder is highly heterogeneous and may arise from several mechanisms. The current research is based on the theoretical model of a social engagement system (e.g., Porges, 2007; Porges & Lewis, 2010), which provides a physiological model that explains a functional role of the MEMs in regulating the spectral content of acoustic information (i.e., selective filtering of acoustic information) received by the first neural transducers (hair cells) of the auditory system.

Tonic MEM tone provides an important first peripheral filter in the processing of acoustic information. An emergent integrated social engagement system occurs in mammalian species due to the common brainstem structures involved in regulating autonomic state via the vagus and the striated muscles of the face and head by feedback via several cranial facial muscles (Porges, 2007; Porges & Lewis, 2010). The MEM and the regulation of MEM tone is a component of this integrated social engagement system. Thus, MEM tone, similar to vocal prosody, should parallel autonomic state (i.e., vagal regulation of the heart). While the circuit responsible for reflexive contraction of the middle ear muscles is well defined, it does not account for the multiple synaptic projections to the motorneuron pool of either the stapedius or the tensor tympani (Mukerji, 2010). Descending pathways from the locus coeruleus, inferior colliculus, and superior olivary complex are either directly or indirectly connected to the stapedial motorneuron pool (Rouiller, 1989; Brown, 2008).

Hypersensitivity to sound, particularly to low-frequency sounds, provides an advantage to mammals in the wild by increasing the likelihood that they will detect an approaching predator (Porges & Lewis, 2010). However, in safe environments, mammals forego this defensive state and focus on the vocalizations of social communication that are characterized by low amplitude higher frequencies. Humans may maintain the ability to modulate their auditory system into this type of profile (more sensitive to frequencies below 1000 Hz, less to those above) as a response to threat. Similar to the inhibition of the sympathetic (i.e., fight or flight) component of the autonomic nervous system by the vagus in safe environments (Porges, 2007), the auditory system is ‘tuning out’ the low frequencies in safe environments.

However, a disordered neural system, due to infection, damage, or neurophysiological state, may alter the sensitivity to sound by disrupting the normal resting tone on the middle ear muscles. It should be noted that the middle ear muscles apply a tension to a constant load, due to the negative air pressure within the middle ear cavity. A middle ear with little tension would by hypersensitive to low-frequency sound and at a disadvantage for detecting the frequencies above 1000 Hz. For humans this would result in a hypersensitivity to background noises and a hyposensitivity to the frequencies associated with human voice. Research in cats on the acoustic stapedial reflex has indicated some role for context in determining the behavior of the middle ear muscles (Simmons & Beatty, 1962).

Human Vocal Communication: If context modulates MEM tone by changing the relative contribution of frequencies above and below 1000 Hz to the signal received by the cochlea, as proposed, a benefit of high MEM tone would be to facilitate comprehension of vocal communication. Human vocal communication utilizes complex acoustic signals, a combination of spectral components that change in pitch and amplitude over time, often in a multimodal fashion (i.e., the components behave independently to some extent). When a person speaks or sings, their voice contains a fundamental frequency referred to as the pitch. However, the energy of a spoken word is also spread across higher frequencies, with maximal energy near harmonics of the fundamental. The higher frequency harmonics (i.e., formants) enable the accurate detection of words.

A simple phoneme, a single syllable of only 40 ms, the sound “da”, may have a fundamental that rises along with higher formants, which will rise, fall, and maintain pitch. These language related processes (i.e., the production of a fundamental and higher frequency harmonics) occur within a frequency band from 103 Hz to 4500 Hz. The second through fifth formants span 1240 Hz to 4500 Hz (from Hornickel, 2009). The spectrograms of stimuli used in the speech intelligibility task in the current example (see below, Numbers in Noise) are even more complex than this simple syllable. The precise frequency range of any formant is impossible to define, because it is a function of the pitch of the speaker, as well as other characteristics (e.g., body size) that determine the resonances of the speaker's voice production system.

While there are differences in pitch and individual formant location (in the frequency domain) among genders, languages, and racial groups, they all fall within the range of frequencies for which auditory sensitivity is greatest. This bandwidth of interest in all vocal comprehension closely matches that reported by Hornickle (2009) in the example of a synthesized phoneme provided above. The higher formants assist the cognitive process of speech intelligibility, because they convey overlapping information from the fundamental and reside in a more sensitive region of the auditory spectrum and use downstream mechanisms that are sensitive to the slight variations in acoustic energy in this restricted frequency band. The formants serve an additional purpose in human communication by conveying contextual information, such as by placing emphasis on a word, syllable, vowel, or consonant (Erickson, 2002).

Laterality: Some functions of the auditory system are lateralized. For instance, dichotic speech-like sounds presented to each ear are more correctly identified from the right ear than the left ear (Hugdahl, 2001). The intensity difference in the dichotic pair required to overcome this bias is between 6 and 9 dB (Hugdahl, 2008). However, this advantage is known to be modulated by attention (Voyer, 2005). Interestingly, this dichotic difference changes with age, as the forced left ear response paradigm does not decline, but the forced right ear response paradigm does decline with age (Hugdahl, 2009).

Lateral differences exist before the level of word comprehension (i.e., lower in the signal transmission pathway). Auditory evoked responses show differences between subjects in left temporal lobe latencies and amplitudes (Ahonniska, 1993). These differences are seen even at the level of the auditory brainstem response (Levine, 1988; Sininger, 2006; Hornickel, 2009). The acoustic startle reflex has an asymmetric representation with brainstem recordings as well (Kofler, 2008).

Middle ear muscle tone during quiescent state is linked to autonomic state as a special visceral efferent component of the social engagement system (Porges, 2007). The autonomic nervous system is itself highly lateralized. The organs are not oriented symmetrically, and the neural networks that regulate their function are similarly lateralized. Vagal control of the heart, via myelinated pathways descending from the nucleus ambiguus, is right biased (Porges, 1994).

The separated bone structure of the mammalian middle ear is a defining feature of the genus in the fossil record (Wang, 2001). Although the evolutionary pressure responsible for this adaptation is still disputed (Rowe, 1996, Wang, 2001), the structure of the ossicles contributes to the overall transfer function of the auditory system by compression of low-frequency sound intensities and facilitating the decoding of higher frequency information (Zwislocki, 2002).

The ossicles do not vibrate with the same movement for all frequencies in the auditory bandwidth of perception (Decraemer, 1991; Willi, 2002; Stenfelt, 2006), which is roughly 20 to 20,000 Hz in humans. These separate modes of vibration impact on the transfer function of the ME, creating a mismatch between the impedance for a pure tone and the impedance for that same tone paired with another tone (if the second tone resides in a different vibration mode).

The Middle Ear Muscles: The transfer function of the middle ear defines the translation of airborne vibrations to fluid waves transmitted to the cochlea through the oval window. The stapes is the final ossicle in this transmission path, directly contacting the oval window. The stapes is bound to the middle ear cavity by the stapedial muscle, one of two muscles of the middle ear. The second muscle of the middle ear is the tensor tympani, which is considerably longer than the stapedial muscle and is attached to the first ossicle in the sound transmission path, the malleus. The tensor tympani is also implicated in the regulation of the Eustachian tubes. The tensor tympani muscle is innervated by fibers from the trigeminal nerve (CN V) and stiffens the ossicle chain during chewing, swallowing, and vocalization. The stapedius is smaller and connects the stapes to the outer wall of the middle ear cavity. The stapedius is innervated by a branch of the facial nerve (CN VII), and is known to reflexively contract in response to loud sounds. Both of the middle ear muscles are innervated bilaterally, so that contraction on one side of the head co-occurs with contraction on the other side in a healthy system.

The middle ear is connected to the sinus cavity by the Eustachian tube. Ear infections, the most common middle ear disorder among children, can occur when the Eustachian tube closes and fluid builds up behind the ear drum. While the tensor tympani does not directly regulate opening of the tube (Honjo, 1983), a branch of the trigeminal nerve also innervates the tensor veli palatini, and both muscles are implicated in Eustacian tube functioning.

The transfer function of the resting middle ear is a function of the geometry and physical characteristics (e.g., stiffness) of the component parts (e.g., bones, tendons, muscles). This transfer function is changed by the reflexive contraction of the stapedius muscle (Liberman, 1998; Pang, 1997).

Middle Ear Muscle effect on Energy transmission: It has been reported that filtering at the level of single auditory nerve fibers, due to electrical stimulation of the stapedius, is linear with relation to the amplitude of the electrical stimulation (Pang, 1989, 1997). Pang reported in the cat that electrical stimulation of the stapedius resulted in a flat attenuation for frequencies below 1000 Hz of 20 dB, a flat attenuation of 8 dB for frequencies above 6000 Hz, and a sigmoidal slope from 1000 to 6000 Hz (Pang, 1989). Contraction of the tensor tympani alters the transfer function of the middle ear in a different manner, although it also serves to attenuate the transmission of low-frequency energy into the cochlea. The attenuation provided by the contraction of the tensor tympani muscle is most effective at attenuating the transmission of bone conducted sounds, including those made by chewing (Irvine, 1976). However, the stapedius muscle is also recruited in some situations that require attenuation of bone conducted internal sounds, such as during vocalization (Borg, 1975).

Nonuniform (with respect to frequency) changes in energy transmission could facilitate the detection of higher frequency sounds in the presence of low-frequency noise (Pang, 1989, 1997; Borg 1972b). In single auditory-nerve fiber recordings in an anesthetized animal preparation, Pang showed that the contraction of the stapedius could interact with acoustic stimulation to ‘unmask’ a high tone, 6000 or 8000 Hz, in the presence of a low-frequency, broadband masker (i.e., noise). In terms of the individual fibers firing rate, the unmasking could be as great as 40 dB (Pang, 1997). This study used electrical stimulation of the stapedius muscle and further demonstrated that the degree of contraction correlated with the degree of unmasking (i.e., the greater the tone of the muscle, the greater the unmasking).

Contraction of the middle ear muscles by individuals capable of voluntary contraction indicate that the response acts as a high pass filter, reducing transmission by 30 dB at 500 Hz, with virtually no effect at 1000 Hz (Kryter, 1986). If high pass filtering is occurring within the middle ear at intensity levels typically encountered in the environment, the resting tone of the middle ear muscles could play a crucial role in extracting the frequencies associated with human voice from lower frequency noise. This type of active filtering also implies that individuals with high resting tone will perceive a difference in relative intensity of low and high notes that is greater than an individual with low resting muscle tone.

Measuring Aspects of the Acoustic Transfer Function: Psychoacoustic studies confirm the nonuniform nature of auditory perception (Fletcher & Munson, 1933). Loudness perception varies as a function of frequency, and the shape of this ‘equal loudness’ curve flattens as intensity increases (Suzuki, 2004). All mammals have an increased sensitivity (i.e., lower threshold for detection) for a range of sound frequencies used to socially communicate with conspecifics (Porges & Lewis, 2010).

Fletcher and Munson contribute to the expansion of the field of psychoacoustics. Within this discipline, studies are conducted to evaluate subjective perceptions when acoustic stimuli (e.g., pitch, intensity, etc.) are manipulated. In the discipline of psychoacoustics, perception is measured via self-report. Resting MEM tone is hypothesized to impact the functional output of the hearing system and thus the perception of loudness as a function of frequency. This was tested by evaluating the covariation between energy reflectance from the ear canal and individual differences in loudness scaling as measured by the equal loudness contour.

Clinical inspection of the middle ear has centered on identifying common conditions that disrupt the mechanical operation of the vibrating ossicles. The ossicles reside in a gas filled compartment, connected to the sinus cavities by the Eustachian tube. Tympanometry is used clinically to test the compliance of the tympanic membrane, by modulating the air pressure external to the middle ear (i.e., in the ear canal). In this way, perforations of the ear drum and the presence of fluid in the middle ear cavity (i.e., an ear infection) can be detected in most cases. Comparison of air conduction and bone conduction sound thresholds is also used to detect discontinuities in the ossicle chain (i.e., broken bones) or fixation of the ossicles, otosclerosis.

The Zwislocki Bridge (Burke, 1967) is a major advancement in studying the transfer function of the auditory system, particularly the middle ear. This device allows a researcher to balance the impedance of the middle ear with parallel impedance. Thus, with an acoustic analogue of a bridge circuit, small changes in the impedance of the ear could be detected. This allowed more reliable detection of the ASR threshold by the smallest noticeable change in the impedance of a test tone, played into the ear canal through the bridge. Advances in this type of acoustic immittance testing of ASR thresholds allowed clinicians to reliably measure threshold both contralaterally (i.e., in the ear opposite the reflex inducing sound) and ipsilaterally (Lutman, 1980).

Advances in digital signal processing allowed greater precision in wideband measurements of acoustic impedance (Keefe, 1993; Allen, 1994; Feeney, 2004). This approach determines the source impedance of a probe (consisting of a microphone and a speaker) and an estimation of the characteristic impedance of the ear canal by an estimate of the ear canal shape. Combining this information with the frequency characteristics of a reflected broadband signal, an estimate of the impedance, reactance, and reflectance of the static middle ear can be obtained.

Tympanometry employs a probe tone (usually 226 Hz), the impedance of which is measured continuously as the ear canal pressure is modulated. The clinical utility of tympanometry in detecting abnormalities in newborn middle ears is significantly worse than for adults (Rhodes, 1999). An attempt to improve the utility of the tympanometric procedure was made by employing multiple probe tones at different frequencies (Colletti, 1976, 1977). Margolis continued to use multiple probe frequencies in tympanometric analysis with greater success in children compared to single tone analyses (1985, 1993, and 1994). Both wideband measures of the static middle ear and multifrequency tympanometry have demonstrated clinical utility in identifying disordered middle ears from normal healthy one (Margolis, 1994; Shahnaz, 1997; Beers, 2010).

The existing techniques for measuring wideband energy transmission in the middle ear are sufficient for diagnosing several middle ear disorders. By establishing normative datasets, diseased middle ears can be distinguished from healthy ones by comparison. Further, the features associated with certain disordered states, such as increased stiffness in the presence of otitis media, can be distinguished by these methods. The broad question asked by clinicians using these devices is: What, if anything, is wrong with this middle ear? This example, in contrast, examines the functional impact of resting middle ear muscle tone on hearing and listening. The measurement provided herein classifies healthy middle ear systems along a continuum of resting muscle tension.

MESAS: To increase the understanding of variations in the healthy intact middle ear, this example employs continuous stimulation via probe tones (as in multifrequency tympanometry) across a wide range of frequencies that overlap with the bandwidth of increased absorption by the middle ear (as in wideband reflectance). The selection of the range of frequencies for analysis in this new measure is motivated by the spectral content of human speech and the known influence of the middle ear muscles on energy reflection at the tympanic membrane.

Existing measures of ME power flow characterize the transfer function at the point of the eardrum. By utilizing brief signals of isolated frequencies (chirps) or broadband waveforms (clicks) they measure the reflection at the eardrum of a single frequency at a time. In contrast, we consider the behavior of a middle ear vibrating at frequencies that span the range of vocal communication. In developing the probe stimuli, two physiological factors inform the decision to consider the continuous broadband signal for the reflectance measure: 1) the ear canal provides a significant amplification to acoustic information, and 2) the middle ear muscles are constantly applying tension to the ossicles (possibly only at the stapes, but likely also at the malleus through the tensor tympani).

This interaction between the middle ear muscles and the ossicle chain should alter the resonance of the ear canal. This interaction is further complicated by the multiple vibration patterns of the ossicle chain. Since the vibration mode of several ranges of frequencies apply different forces to the middle ear muscles (Decraemer, 1991), it is proposed that the impedance for a given frequency “X” depends on the presence (and likely the intensity) of vibration at frequency “Y”.

Thus, traditional measures based on the reflection of impulses, such as in wideband measurements of acoustic power flow (e.g., Keefe, 1993; Feeney, 2004; Allen, 2005) continue to be the ideal way to characterize the transfer function of the eardrum specifically (i.e. in a linear interaction with a single frequency). Those methods, by stimulating the ear canal with short duration bursts, do not allow the ear canal resonance to influence their measurements. Also, those methods eliminate information regarding the reflectance of frequency “X” that is due to tension in the middle ear muscles when vibrating at frequency “Y.” The methods provided herein contribute additional information about the energy absorption of the active middle ear. The results are not directly translatable to the measures of acoustic power flow without collecting measurements on both systems and estimating a transfer function between the two. To distinguish between this new measure of energy reflected from the occluded ear canal and other currently used measures of wideband reflectance, in this example this new measure is referred to as middle ear sound absorption (MESAS).

Specifically, it is the transfer function relating the input energy of the acoustic signal to the incident energy measured at the end of the occluded ear canal when the ear canal is continuously stimulated by a range of narrow frequency tones. The MESAS unit is decibels. The measure of gain at each frequency in the narrowband probe is normalized by the gain at 1000 to yield a ratio, the metric used in this example.

Studies based on the device and method applied here attempted to measure individual differences in the time course of MEM changes in response to acoustic stimulation at intensities below the acoustic reflex threshold. MEM tension may be modulated rapidly based on the acoustic environment and remains relevant.

The acoustic startle response includes an eyeblink component in which the muscles that close the eyelid oppose tension from the orbicularis oculi. Greater resting muscle tone in the opposing muscle reduces the latency of the reflex (Hawk, 1992). Prepulse inhibition, the classically conditioned reduction of the reflex magnitude following trials where tones precede the stimuli, is slower in autistic individuals (Perry, 2007). Autistic individuals typically have reduced muscle tone to the facial muscles, particularly the muscles of the upper face innervated by the facial nerve. The neural regulation of these facial muscles is an example of another special visceral efferent component of the social engagement system (Porges, 2007).

The middle ear muscles and startle responses are examples of responses to incoming stimuli, receiver behaviors. Other special visceral efferents are proposed to regulate laryngeal muscles responsible for aspects of vocal communication, sender behavior. Thus the social engagement system is proposed to involve feedback within and between individuals in communication (Porges, 2007). It may be possible to measure the dynamic behavior of the middle ear muscles in a social exchange with the current system. Further research with the described technology will facilitate studying the interaction between resting middle ear muscle tone and dynamic responses to acoustic and nonacoustic stimuli.

Equal Loudness contours: Individual differences in perceived loudness of pure tones (i.e., the equal loudness contour) is measured. This is justified, because psychoacoustic measures are an ideal indicator of the overall effect of the auditory system on a single parameter of a sensory stimulus. In this case, the relative loudness of various frequencies of pure tones is measured. The ME is only one stage in a multilevel filtering process within the auditory system, and as such it only conveys a portion of the overall shape of the equal loudness profile. Individual measurements on the contour are assumed to represent a significant degree of variance between subjects due to the influence of medial olivary cochlear filtering mechanisms, individual differences in auditory nerve density in the handful of fibers excited by the pure tone, and individual differences in test taking behavior. However, the average response to low-frequency tones (below 1000 Hz), in the region most attenuated by MEM tension, and the average to mid (1250 to 4000 Hz) frequency tones, in the region least attenuated by MEM tone provides information on any effect of MEM tone on the individual's perception of loudness.

Numbers in Noise: Existing tests of word intelligibility in the presence of noise are designed to explore integration of narrowband speech in broadband noise, a task that depends on the performance several complementary filters in the auditory system: ME structures, MEM tone, medial olivary cochlear filtering, sensitivity, and brainstem integration of multiple cochlear nerve unit. Above this point in signal transmission, cognitive processes determine some aspects of performance. The Numbers in Noise task is designed to specifically challenge the proposed mechanism of ME filtering as a function of variable MEM tone. Consistent with the suggestions of Liberman and Guinan (1998), the noise is band limited to the frequency range significantly attenuated by tension in the MEMs. The signal is broadband, with information in the higher formants that should aid intelligibility when the fundamental and lower formants are masked by the noise. The stair-step (or up-down) procedure of the test quickly converges on a reliable estimate of one measure of noise tolerance, the magnitude of noise at which the likelihood of correctly identifying the spoken number is 50%. This parameter is normally distributed in this healthy sample with normal hearing.

MESAS: First, within a restricted sample of normal hearing adults, an attempt is made to validate the role of the MEMs in listening. By focusing on the small range of differences encountered in a healthy population, the power of any observed relationships is reduced. However, this conservative approach means that any findings should reflect phenomena likely magnified in clinical populations with difficulties in speech recognition or hyperacusis.

Initially, the dynamic motion of the middle ear through phase changes in the continuous probe signal is measured. This measurement may be beyond the sensitivity of the devices. Next tested is the hypothesis that the magnitude of the reflected energy from the ear canal reflects individual differences in resting MEM tone. The continuous probe signal is used to fully exert the muscle components of the middle ear during measurement by fully exciting the ossicles. This reflection magnitude should mirror the absorption of energy into the sense organ of the cochlea for transduction into neural impulses.

Second, conventional existing technologies for middle ear power analysis (i.e., impedance or reflectance) operate by playing and recording short bursts of sound within the sealed ear canal. This technique bypasses the influence of resonance within the ear canal. Measurements with devices based on this technique have clinical applications in diagnosing disorders and screening newborns. However, the experience of listening to a click train of 65 dB SPL at 8 to 20 clicks per second is not pleasant. One design criterion for this measurement system was that researchers must be able to use it in a population of ASD children and adults, some of whom have severe auditory hypersensitivities. Therefore, one objective is a test experience that is as nonabrasive as possible.

Lateral Measurements: Cortical processing of speech information is not independent of the transducer of the information (i.e., which ear hears the signal). There is a clear right ear advantage for dichotic words and brainstem differences in speech encoding. There is more efficient neural transmission from the right ear auditory nerve fibers to the left hemisphere language processing centers, since the density of contralateral connections is greater for the contralateral side of the auditory cortex. The neuroanatomy, neurophysiology, and the functional lateral differences in auditory perception, suggest that information may be encoded differently in the right and left cochlea, or within the first synapses, in order to facilitate features of each hemisphere by providing the cortical structures with the most relevant information. Therefore, the same test could have a different dependence on MEM tone in each ear. Furthermore, the autonomic nervous system is highly lateralized. Therefore, this component of the auditory system (i.e., MEM) is state dependent and sensitive to social context. Thus, there may be within-subject variance in MEM tone (i.e., right or left side more tense) that can only be captured by analyzing both ears separately for all tasks.

Frequency Band of Analysis: The summary statistics span a low-frequency range below 1000 Hz and a mid-frequency range determined by the average location of the second through fifth formants of human speech of 1250 to 4500 Hz. It is the absorption of these signals that is a necessary first step in the auditory system's processing of human language. Since the equal loudness contour has been standardized as a comparison of pure tones to a reference at 1000 Hz, and since 1000 Hz is close to the highest frequency receiving the greatest attenuation by reflexive contraction of the MEMs, the measure is normalized as the magnitude relative to 1000 Hz. Other conventions may be acceptable, but this implies the domains of measurement by keeping the center point consistent. It is assumed that this provides maximum separation between MEM associated changes in energy reflectance, which should be greater below 1000 Hz than at any point above 1000 Hz. The middle ear bones have a resonance near 1000 Hz (Homma, 2009), making this spectral region efficient for vocal communication (i.e., less intensity relative to other frequencies required to be detected).

Questionnaire Data: Subjects are given two written assessments designed to screen for hyperacusis. Each measure provided a numeric score for responses to statements concerning discomfort around noise. Schutte's instrument (2007) includes a mix of both positive and negative statements, while the Khalfa's questionnaire (2002) is structured as queries. The Schutte measure is further divided into subscales based on the environment associated with the sensitivity (i.e., leisure, work, habitation, communication, and sleep). An example from the Khalfa measure (Question 5 of 14): “Do you have difficulty listening to conversations in noisy places?” No; Yes, a little; Yes, quite a lot; Yes, a lot. An example from the Schutte measure (Item 13 of 35): “I need quiet surroundings to be able to work on new tasks.” 0=strongly disagree; 1=slightly disagree; 2=slightly agree; 3=strongly agree.

Questionnaires are scored and the total used as an additional measure of perceived auditory sensitivity. Both devices are designed to make a binary decision for clinical treatment of hyperacusis, so the application of the composite score is exploratory.

Computer Based Psychoacoustic Measurements: Subjects first complete a set of psychophysical tests based on custom code written in MATLAB. Subjects sat at the PC, and were equipped with Sennheiser HDA-280 headphones. These research headphones have a flat magnitude response from 20 Hz to 800 Hz, and less than 10 dB attenuation up to 12,000 Hz, appropriate for audiometric testing. Psychoacoustic measures are designed to test two hypothesized functional outcomes of MEM tone: perception of loudness and hearing in noise. All computer based testing is performed in custom-written MATLAB® software. Each test is designed to answer a theoretically motivated question about the functional significance of resting MEM status on hearing. Since each test is performed monaurally, the procedures allow the evaluation of laterality differences in both loudness perception and the performance identifying words in noise. All tests are presented using a graphical user interface (GUI) designed in MATLAB®.

Calibration: Psychoacoustic tests are performed with over the ear headphones (Sennheiser). Prior to testing, the stimulus intensity is normalized across subjects by the presentation of a calibration signal, a pure tone at 1000 Hz. The intensity of this signal is verified to be 120 dB sound pressure level (SPL) on a sound level meter coupled to the headphone's right ear piece by a sound isolation device (i.e., modified hockey puck). The headphones are placed flat on a table with the earpiece facing upwards. The sound level meter is placed completely over the earpiece and the tone played. The sound level meter measures intensity on an A-weighting (i.e., dB(A)). However, the three loudness scales commonly used: A, C, and SPL are all normalized at 1000 Hz. So, 50 dB(A) at 1000 Hz is equivalent to 50 dB(C) and 50 dB SPL. The SPL scale represents the true intensity of the acoustic signal, while the A and C ratings are designed to bring approximate loudness as perceived by humans. The researcher makes fine adjustments to the sound intensity on the preamp (Behringer, Inc.) in order to obtain a proper calibration (less than +/−0.5 dB SPL).

Numbers in Noise test: The numbers in noise (NiN) test is designed to maximize the relationship between performance (i.e., noise tolerance) and the theorized impact of MEM tone on sound absorption. For this reason, the competing noise is band limited to frequencies below 650 Hz. Increased tension in the MEMs should decrease the absorption of this low-frequency energy. The speech component is generated by a text-to-speech program (Microsoft) with a synthesized female voice. The higher fundamental frequency of this “voice”, compared to the noise content, meant that increased tension in the MEMs should facilitate absorption of the speech signal, functionally increasing the separation between the numbers and the noise. 10 recordings of test-to-speech numerals (0-9) are saved for use by the testing program. The quality of these recordings is 44,100 Hz and are saved as uncompressed way files.

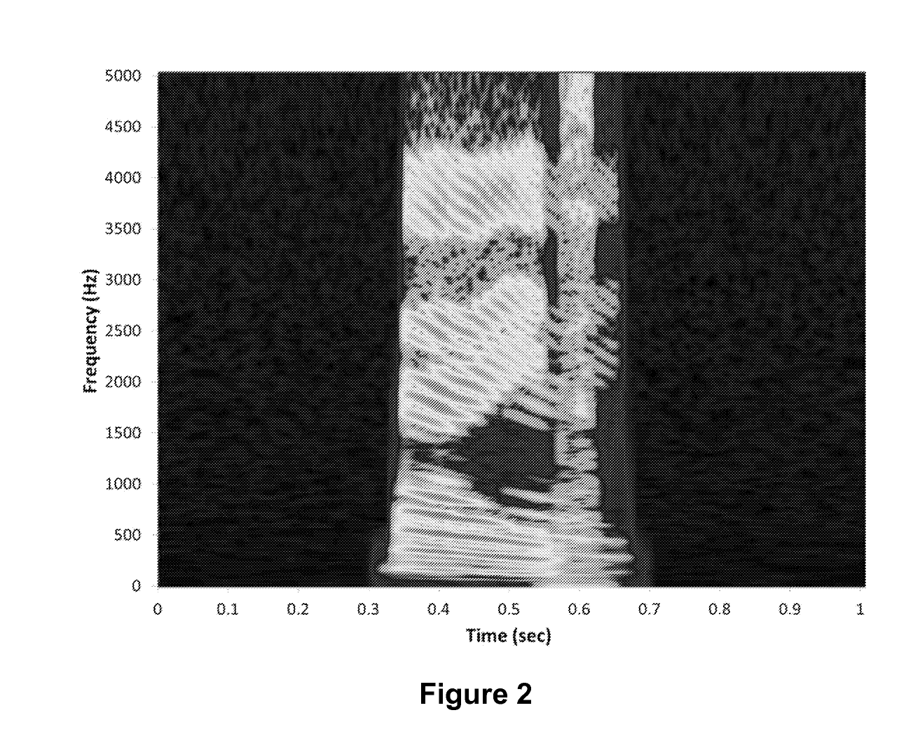

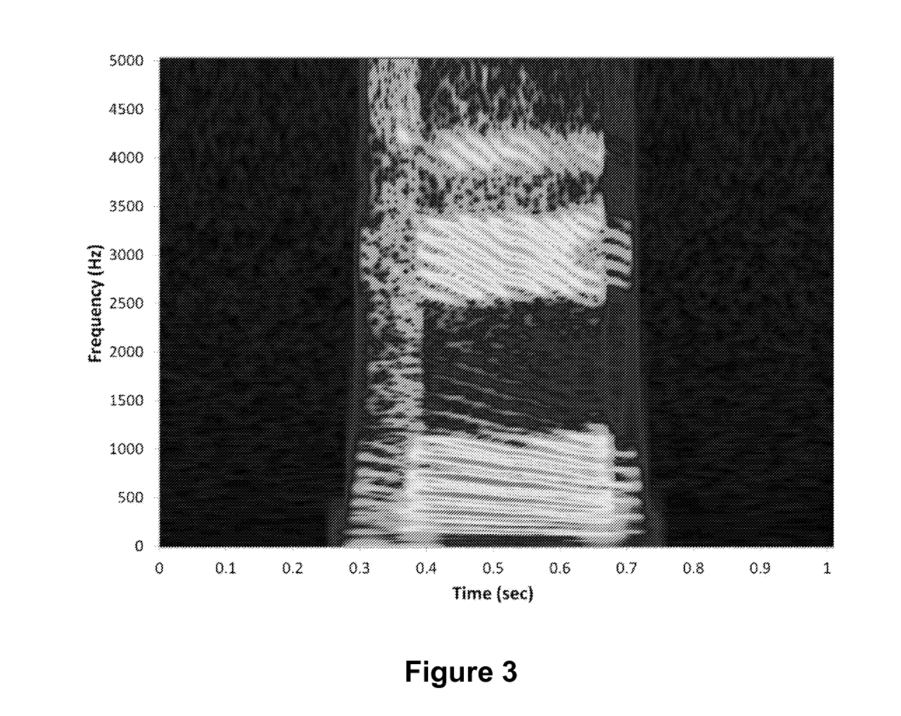

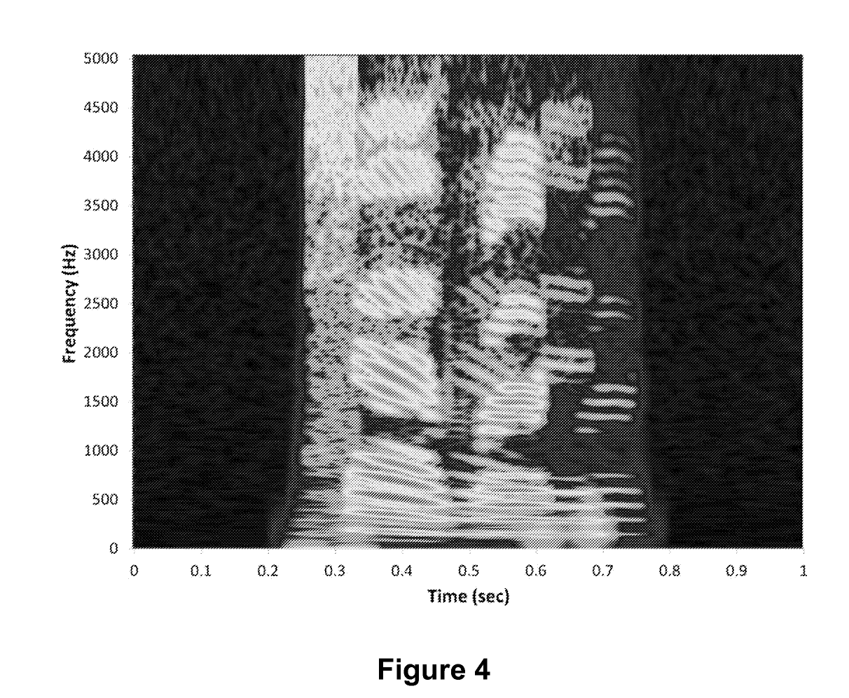

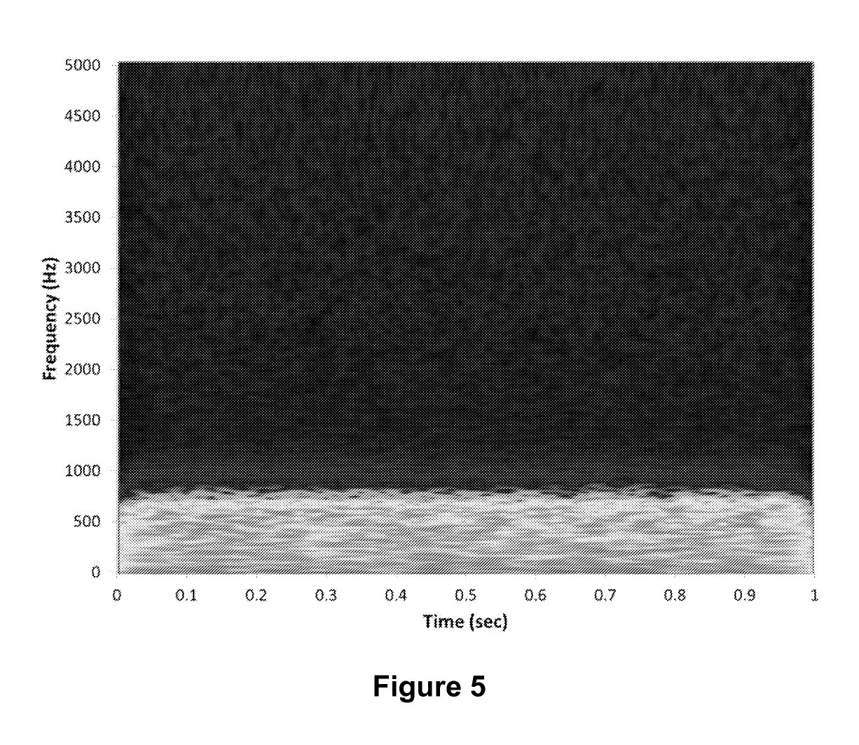

The spectrograms of FIGS. 2-5 illustrate the higher formants, which extend up to about 4500 Hz, of the synthesized speech signals in agreement with the measurement range of MESAS.

The noise component of the signal is generated by Adobe Audition® 1.5 (Adobe, Inc.). This signal is pink-noise, with a frequency content that closely matches the spectral envelope of the natural world. Pink-noise has a low frequency roll-off that approximates a 1/f distribution, where f is frequency. This is in contrast to white-noise which has a flat spectral envelope (i.e., uniform distribution) and “random-walk” brown noise which is more biased to the lowest frequencies with a 1/f² spectral envelope. The pink-noise is then low-passed filtered with a 10^{th} order Chebychev Type I filter. The final noise mask consistently covers the fundamental of the speech signal and usually the first harmonic.

At the start of the NiN test, the subject is presented a simple instruction through the GUI. The subject heard a composite of a random numeral (approximately 30 dB(A) and the initial level of noise (approximately 40 dB(A)). The subject is instructed to press the number on a keypad that they heard. Each mixed recording begins and ends with noise only. The duration of each numeral is not consistent, but the noise recording is longer than the longest numeral recording.

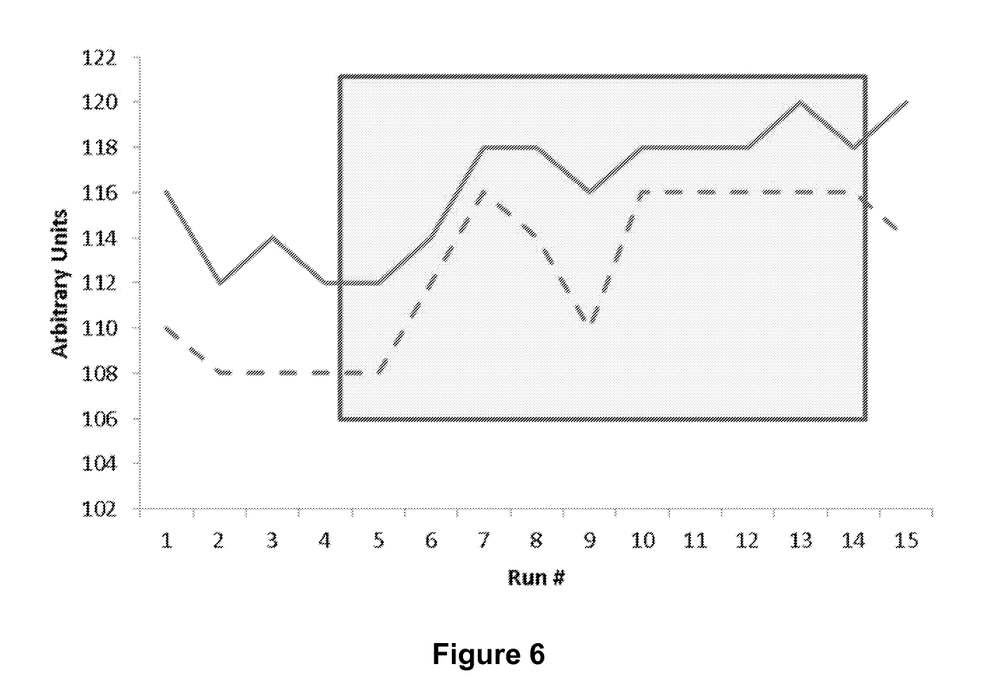

With each presentation of a number in noise, the subject was asked to press the number on a number pad, if it could be discerned. Correct responses increased the noise level in the following recording, and incorrect responses decreased the noise level, while the intensity of the computer generated voice was held constant. After an initial run of three correct responses, the level change parameter was reduced to 2 dB. At the end of each run of correct or incorrect responses the noise level was recorded. The last run was excluded from analysis due to an error in some trials that recorded this value as 0. The noise intensity level at which there was fifty percent detection was estimated from the last ten high and low levels by the up-down or staircase method (Levitt, 1970). This measure was termed NiN_{—}50. Each test lasted between five and ten minutes in each ear. The NiN_{—}50 value is the mean of the maxima and minima shown in the box of FIG. 6.

Equal Loudness contour test: This psychoacoustic test is based on the equal loudness contours described by Fletcher and Munson (1939). As is standard for this test, the perceived intensity of pure tone stimuli is compared to a calibrated 1000 Hz reference tone presented at 60 dB SPL (Suzuki and Takeshima, 2004). The computerized implementation of the equal loudness contour measurement is named EqL. In it, subjects heard the reference tone for one second, followed by the test tone for one second, repeating this pattern until the subject made an input. An indicator in the GUI informed the subject when the test tone is presented. Subjects have a choice of keyboard or mouse control over a volume slider to change the intensity of the test tone. While making adjustments to the intensity, the test tone is presented continuously. The stimulus presentation returned to the alternating pattern when the subject stopped moving the volume slider. The subject pressed a button in the GUI when satisfied that the two tones had equal loudness and received the next in a series of 17 tones (31.5 Hz to 13,500 Hz).

MESAS data are collected on a prototype system developed at the Brain-Body Center (Chicago, Ill.). The prototype incorporates commercially available hardware, custom software, and custom acoustic stimuli into a single measurement system. The main design criteria for development of the system are: (1) reliability, (2) ease of measurement, and (3) suitability for testing challenged populations (e.g., autistic individuals with auditory hypersensitivities). The frequency range of measurement and normalization procedures are adopted based on theory driven motivations.

Stimulus: The stimulus is a custom generated digital audio file (Audition 1.5, Adobe, Inc.). The recording has two parts: a synchronization pulse and a multi-frequency probe tone (also referred herein as a “non-harmonic acoustic input” or “comb input”). Each component is generated with functions built into Audition™. A single, 500 Hz sin wave is enveloped to have two instantaneous transitions from full to zero amplitude. These changes are detected by the recording software and used to truncate the data for analysis. The preceding and trailing 500 ms of the probe tone are excluded from the analysis to assist in obtaining a steady-state response.

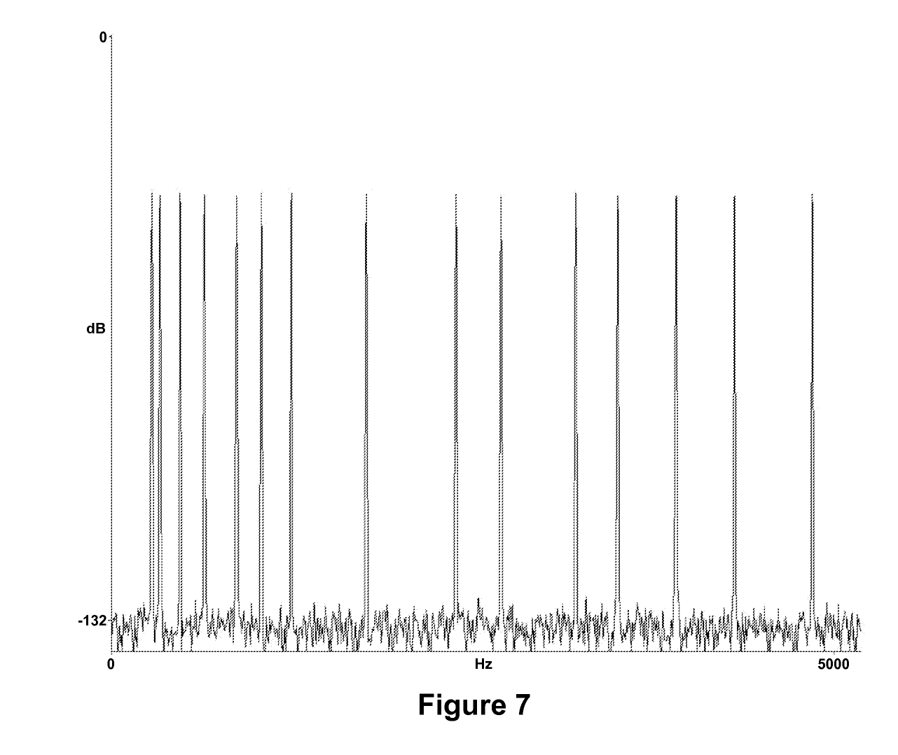

The probe tone is created by mixing three sets of five-tone chords with center frequencies chosen to avoid integer harmonics within the set (FIG. 7). Each component is mixed with equal amplitude into the chord, and the three sets merged by the mixdown procedure. The final recording is verified to contain equal amplitude at each of the 15 frequencies by spectral analysis. Although any number of components having any number of frequencies may be selected, the exemplified embodiment in this example is (in Hz): 280, 336, 476, 644, 868, 1040, 1248, 1768, 2392, 2705, 3224, 3516.5, 3922.25, 4328, 4869 (see Justification of Measures: Frequency bands of analysis). The final probe signal recording is saved as an uncompressed way file with 24 bit precision at 96,000 samples per second. The monaural audio file is 10 seconds long.

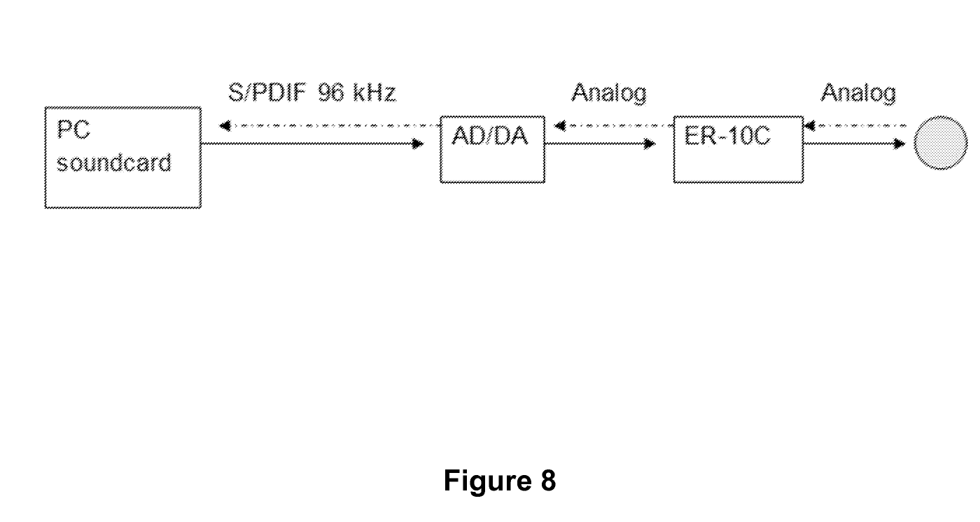

Hardware: The prototype system consisted of the following components: a PC running MATLAB® (r2009a, 64-bit), an M-Audio 192 Audiophile soundcard with 24-bit, 192,000 Hz sampled digital audio with S/PDIF encoding (2-channel, 1-in and 1-out), a Behringer AD/DA and sample rate converter, an amplifier, and an ER-100 OAE preamp and probe assembly (FIG. 8).

A probe assembly designed for distortion product otoacoustic emission stimulation and recordings is connected to the ER-100 OAE preamp. The probe tip contains two sound channels, isolated within a disposable plastic tube attachment that also contains a third larger channel to balance the pressure load on the transducers. The probe tip contains the microphone and speaker transducers.

Software: The probe tone is played through Winamp®, called as a subfunction of the testing software in MATLAB®. Winamp® is modified to apply no amplitude or spectral alterations to the recording and is used to play the probe tones through the onboard M-Audio soundcards digital output at the native sampling rate of the way file (96,000 Hz). The MATLAB® GUIDE tool is used to generate the recording software. The software provides a simple graphical user interface (GUI) in which the user initiates each session by pressing a button, which prompts the user for a unique subject ID for the session. A log file is generated for the session and time stamped with the computer clock's time at that moment. Further log entries are added for each recording initiation.

Calibration: Currently, the user calibrates the intensity of the stimulus before initiating the recording. Alternatively, the device may apply a step-up procedure to probe tone intensity, ensuring a reliable measure is obtained with every replacement of the probe. In this preliminary study, the intensity of the stimulus is calibrated once with the probe in the ear canal at the start of the measurement session. A single 500 Hz sin wave, matched to the intensity of the synchronization pulse of the probe, is continuously output to the probe through Winamp®. The recorded wave was periodically sampled from the digital audio input channel of the sound card and the spectral density plotted in a small window in the GUI. The researcher then adjusts the volume of the probe tone on the AD/DA output until the plotted intensity falls within a range selected to minimize variance in reflected probe intensity across subjects.

Recording: After calibration, the user is provided options in the GUI to initiate measurements. A toggle button allows the researcher to designate in the recording the lateral placement of the probe (i.e., right or left ear). Each recording initiated a presentation cycle consisting of: (1) playback by Winamp®, (2) placing a mark in the log file, (3) recording from the soundcard, (4) analysis of the reflected energy, and (5) visual display of the normalized reflectance curve along with normative data based on previous recordings. The complete presentation cycle lasts approximately 12 seconds. The researcher repeats the recording if the visual interpretation is abnormal or if there are concerns about the placement or seal of the probe.

Analysis: A normalized measure or relative energy reflectance is obtained by a two-step process. Using a function in the MATLAB® signal processing toolbox, spafdr, the transfer function between the output signal and recorded reflected wave is calculated. Each signal is stored in one channel of the digital recording file sent to the PC by the AD/DA device.

The spafdr function is an autoregressive based spectral density function with the ability to specify the frequencies of measurement and the tolerance of each parameter in the polynomial model used to estimate the transfer function. The probe frequencies are used with tight tolerances in order to limit the influence of bodily noise in the reflectance measurement. The transfer function gain values are normalized to create a measure of relative energy reflectance, independent of the total energy reflectance (i.e., the balance of reflected energy as opposed to the level).

In the frequency domain, the stimulus is a narrowband signal composed of 15 equal intensity signals (see, e.g., FIG. 7). The recorded reflection wave is significantly transformed three times, twice by the impedance mismatch between the probe tube and the ear canal (a relative constant between subjects) and upon reflection off the ear drum. The tolerance parameter for each of the probe signals in the transfer function analysis is set to +/−0.1 radians per second in order to minimize the influence of bodily noise on the gain parameter.

Prior to normalization, the gain at 1000 Hz is estimated by cubic spline interpolation from the gain values at the three closest frequencies, 868, 1040, and 1248 Hz. The inclusion of a probe tone at 1040 Hz decreases the variance in this estimation between recordings. Normalization is applied at 1000 Hz to standardize the reflectance magnitude with reference to the psychoacoustic measure of loudness perception (i.e., Equal Loudness contour).

MESAS=10*Log₁₀ (Gₓ/G_{1000 Hz}), where x is the frequency of measurement.

Participants: Study 1. Normative (“Reference”) Data: Twenty-two subjects are recruited through flyers and the University of Illinois at Chicago Psychology student subject pool. Subjects are excluded from the normative dataset if they failed the audiometric screening or a test-retest reliable measure of MESA could not be obtained. One subject withdrew from the study after failing the audiometric screening. One subject pool student chose to complete the protocol despite failing the audiometric screening. One subject reported severe difficulty hearing in noisy environments and was excluded from the normative dataset due to history of hearing difficulties. Two subjects passed the audiometric screening, but were excluded due to inconsistencies in their MESA measures (i.e., failed to get test-retest readings that matched). The final sample for the normative dataset included 17 subjects, with all recordings and measurements performed monaurally in each ear. The normative dataset had an even gender distribution: 8 males and 9 females. The age distribution was both young and homogenous (M=21.6, SD=4.12 years).

Protocol: After institutional review authorization, informed consent was obtained from all participants. All subjects passed an audiogram screening with, at minimum, 50% detection at 500 Hz, 1000 Hz, 2000 Hz (10 dB SPL), and 4000 Hz (5 dB SPL). These frequencies covered the measurement range of interest in this example (500 to 4000 Hz) and are typically employed in screenings for severe hearing loss, especially in the range of human voice.

After the psychoacoustic tests, the researcher adjusts the audio system to be suitable for recording of the MESA reflected energy profile. The subject was seated in front of the measurement system, and a disposable foam probe tip was attached to the ER-10C probe. The probe assembly was attached to the subject's clothing or to the chair in order to minimize movement artifacts in the recording. The researcher compressed the foam tip, asked the subject to swallow (a procedure known to normalize middle ear pressure), then inserted the compressed tip into their ear canal. The researcher only inserted the tip up to the full depth of the tip; however, if the subject was uncomfortable with this depth of insertion, or the ear canal shape made it impractical, the probe tip was only inserted to the depth available. At least a thirty second wait allows the foam to expand and secure the probe in the ear canal.

Calibration then proceeds as described above. Probe intensity is calibrated once, in the first ear measured. Data collected in developing this procedure indicates that above a threshold intensity required for measurement, there is no change in the reflectance profile as intensity increased within a range of approximately 20 dB. Based on this, the intensity is fixed at a level slightly above the average threshold determined during the pilot testing, and kept constant between all measurements in the session. In order to verify the test-retest reliability of the measure, the researcher measures the two ears in a staggered fashion (see example below). The software allows the researcher to visually verify the consistency of the recordings and make additional recordings if needed due to a failure in the recording (i.e., poor fit to probe or movement artifact).

Participants: Study 2. Flexibility in the middle ear muscle system: Response to an auditory intervention. A subject is tested under a separate protocol to evaluate the effectiveness of an auditory intervention (The Listening Project, Brain-Body Center, Chicago, Ill.) on hyperacusis in autistic individuals (“therapeutic intervention”). This subject attempted to complete the computer based training, but difficulties in understanding and following the instructions precludes inclusion of the psychoacoustic data in this example.

The Listening Project protocol is worth describing in order to understand the time course of these recordings. The subject arrived for pretesting that included continuous measurement of autonomic functions (e.g., heart rate and heart rate variability), the Peabody Picture Vocabulary Test, the Kaufman Brief Intelligence Survey, and a dynamic facial affect recognition task (DARE, BBC, Chicago, Ill.). Prior to these tests, the subject participated in the MESAS measurement. The subject was cooperative, compliant, and eager to see the results of his tests. The remainder of the pretesting was performed elsewhere. Following the pretesting, the subject received the first of five days of a therapeutic intervention auditory in nature. The intervention is a mix of music and spoken word stimuli, digitally processed to enhance the acoustic features of prosody in the original recordings. Each session lasts between 45 and 75 minutes, for a total duration of seven and n half hours of listening. The intervention is always presented in a safe, quiet environment at a low intensity. These features are theorized to provide the environmental platform necessary to engage the social engagement system. The amplified prosodic features in the auditory stimulus is theorized to trigger central feature detectors in the nervous system to facilitate pro-social neural regulation of the striated muscles of the face and head through the social engagement system (i.e., increased resting tone of the middle ear muscles).

The subject participated in a two-month follow-up visit to assess the stability of changes seen following the one-week intervention. At this one-day follow-up, the subject first received an MESAS measurement, and then repeated some of the cognitive and affective testing. After testing, the subject listened to the final day of the Listening Project intervention, and then repeated the MESAS measurement.

RESULTS: Study 1: Normative data are collected in a gender balanced sample of healthy young people without sensorineural hearing loss. Novel measures of spoken word comprehension in the presence of background noise (NiN), and energy reflectance by the middle ear (MESAS) are described. A measure of loudness scaling, based on the well-established equal loudness contour (EqL), is also collected along with two self-report measures of sensitivity to noise. Both of these measure have been validated (Khalfa, 2002; Schutte, 2007). Measures are collected monaurally as applicable to examine the interdependence of each of auditory perception: loudness, sensitivity, intelligibility, and energy transfer. All statistical analyses are conducted in PASW® Statistics 18 (IBM, Inc.).

Hyperacusis questionnaires: The two questionnaires yield total scores that are significantly correlated between subjects, r(15)=0.64, p=0.006. Each measure is normalized (i.e., transformed to an N(0,1) distribution) for inclusion in a composite. The average z-score for the subject's two questionnaires is taken as a composite hyperacusis score. The composite score, C, is:

C={Z−Score (Schutte Total)+Z−Score (Khalfa Total)}/2

These measures provide a measure of personal comfort within the auditory environment. The range of this measure is enhanced by calculating a composite score of the two interrelated measure of hearing sensitivity. Creation of a composite measure based on independent scales improves the generalizability of the hearing sensitivity measure (Shrout, 1998; Spearman, 1910; Brown, 1910). The generalizability of this measure is validated by the high correlation between the composite score and each of the original scores, r(17)=0.91, p<0.001 for Schutte and r(17)=0.91, p<0.001 for the Khalfa total scores.

Gender differences. There is no significant gender differences in the composite hyperacusis score.

Descriptive statistics: Since the composite score, C, is an average of two normalized distributions, it is also normally distributed (TABLE 1)

Numbers in Noise: Each ear yields a similar distribution of noise tolerance, as measured by the NiN task, t(16)=1.08, p=0.296. As described, the combined NiN stimulus is the summation of a spoken word and a variable intensity noise masker (i.e., noise+constant signal). The NiN_{—}50 value corresponds to the intensity of noise mixed with the signal that should yield a 50% likelihood to correctly identify the spoken number. An independent samples t-test indicated no significant differences between males and females on the NiN task (TABLE 2).

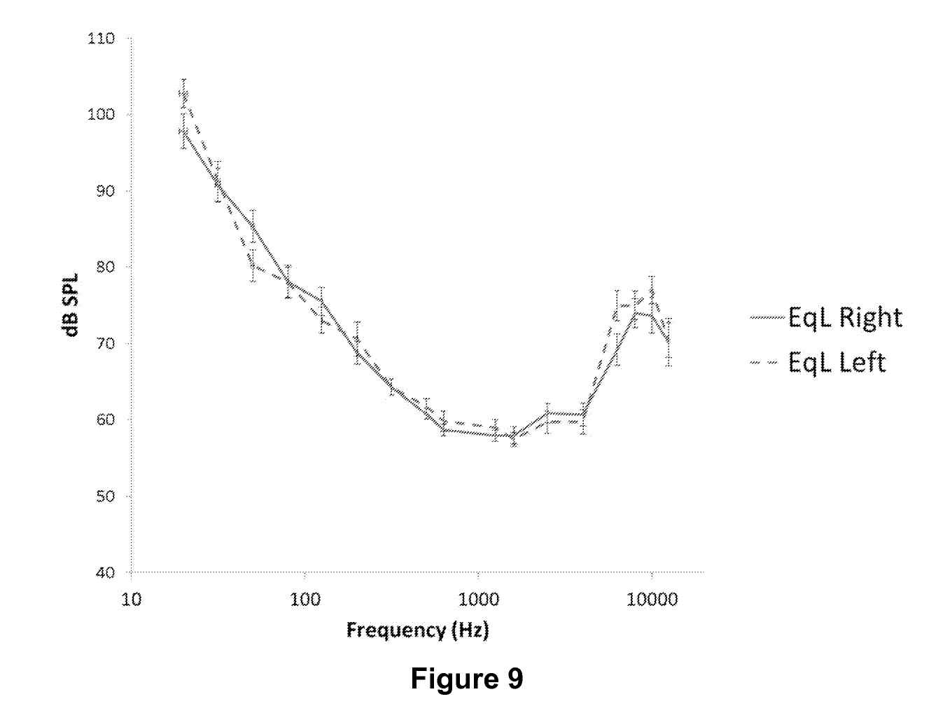

Equal Loudness contour: Laterality differences. A repeated measures ANOVA, with ear and frequency as within-subjects factors, is used to test for laterality differences in the EqL curves (FIG. 9). There is a significant violation of sphericity in this analysis based on Mauchly's test, X² (119, N=17)=193.46, p<0.001. Therefore, the degrees of freedom are adjusted by applying the Greenhouse-Geisser correction, E=0.74. The ANOVA indicates there is a significant main effect for Frequency, F(4.97, 79.5)=65.71, p<0.001. However, there is no main effect for ear, F(1,16)=0.15, p=0.70 and no significant Ear x Frequency interaction, F(6.37, 101.9)=1.92, p=0.081.

Gender differences: A repeated measures ANOVA for both right and left ear EqL responses, with gender as a between-subjects factor, is used to test for gender differences in the EqL curves. There is no main effect for gender in either ear, and no significant Frequency x Gender interaction for either the right, F(1,15)=0.401, p=0.54, or left ears, F(1,15)=0.59, p=0.81. As reported above, there is a main effect for Frequency.

Descriptive statistics: Individual differences at each frequency are normally distributed (see Table 3) with a few exceptions. Due to the broadband effect of MEM tone on energy absorption, summary statistics are calculated based on bandwidths that should show an increase or decrease in absorption relative to 1000 Hz. Since the EqL measure covers a larger range than the MESAS, a high-frequency range (greater than 4500 Hz) is also included for comparison of loudness scaling across this range. Averaging across several frequencies yields estimates that are normally distributed between subjects (see below).

MESAS. The normalized measurement of reflected energy within the ear canal is a novel measure, derived from existing techniques for measuring power flow in the ear canal. Before accepting the output of this technique as a measure of individual differences, it is verified that the recordings provided a reliable measure of individual differences by comparing the test-retest recordings of the right and left ears. As described herein, the ER-100 probe is inserted into one ear, then into another ear, with the researcher looking for a consistent right and left ear profile in the measurement interface (i.e., GUI). Irregular recordings are followed up by checking the setup (e.g., probe securely sealed in ear canal) and repeating the measurement. Subjects without reliable measures are excluded.

Test—Retest Reliability.

A written log of events during the recording is maintained by the researcher. In several instances the researcher failed to indicate the correct placement of the probe (i.e., Right or Left ear), so a feature was added to the analysis software to allow corrections of this parameter. All recordings are reviewed before making the final calculation of the subject's MESA measurement. Recordings are verified for reliability by visual inspection and only excluded if the original researcher notes a problem in the log (i.e., probe fell out) or the reviewer observes one measure that deviates from the pattern of a test-retest pair in that same ear. In the case of a mismatch, multiple MESA recordings had to show a qualitatively similar profile (i.e., maxima, minima, slope) in order to disqualify an outlier recording. This usually occurs when a disruption to the testing session was noted (i.e., probe fell out). The final MESA measure for each ear is the mean of the MESA measures in each accepted trial for that ear.

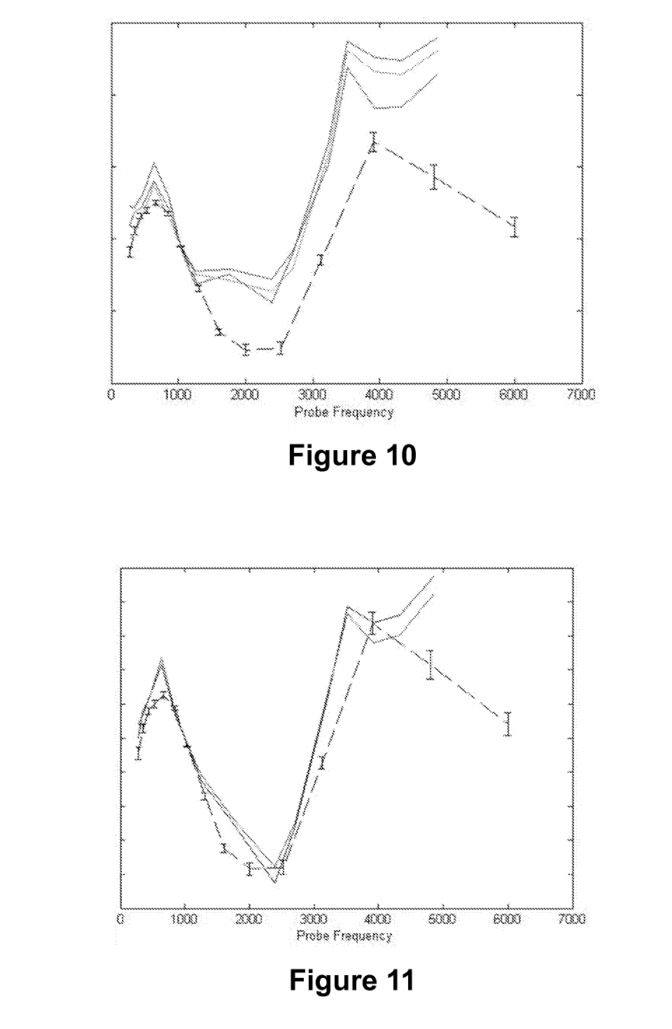

FIGS. 10 and 11 are examples of a typical recording with reliable test-retest, with probe replacement, patterns that are visually distinguishable between each ear. In this case the right ear measure deviated from the normative data, so the initial researcher repeated it. For the third trial, the probe was moved to the left ear. On moving the probe back to the right ear for the fourth trial, the researcher observed the same pattern and was satisfied that the measurement was stable. In the left ear (FIG. 11), the last recording is also a close match to the previous measure in the left ear. The same probe tip is used in both ears, so it is unlikely that this difference is due to the characteristics of the probe itself.

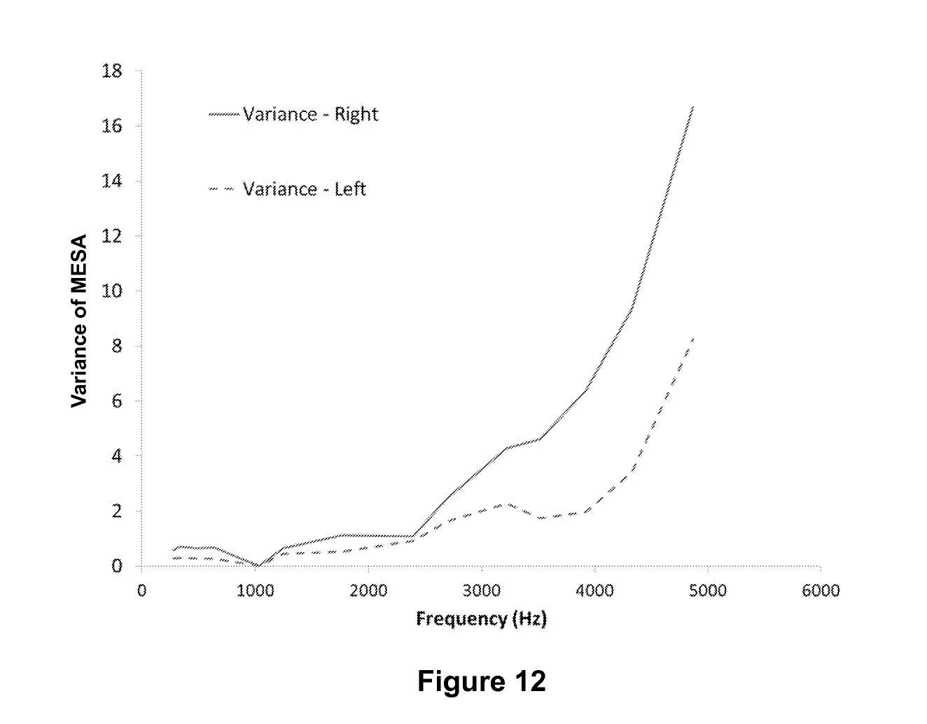

Uneven distribution of variance: The normalization of MESA by the energy reflected at 1000 Hz is adopted to magnify the theorized role of MEM tone on energy absorption in the middle ear. As discussed, contraction of the middle ear muscles stiffens the ossicle chain, increasing the impedance of the middle ear and reducing energy transmission to the cochlea. This attenuation is not consistent across frequencies, with less attenuation above 1000 Hz. The normalization is centered at 1000 Hz, close to the center of the transition point attenuation due to MEM tone (see, Background: MEM effect on energy transmission). This procedure yields a measure that is not consistent in its variance across frequencies (i.e., heteroscedastic) (see FIG. 12). The frequency 1040 Hz is excluded from summary statistics due to its very small variance, and the highest frequency of 4869 Hz is excluded based on it lying outside the frequency band critical to vocal communication. This yields a set of values that are more homogeneous in variance, particularly within the two bands from 280 to 868 Hz and from 1248 to 4328 Hz.

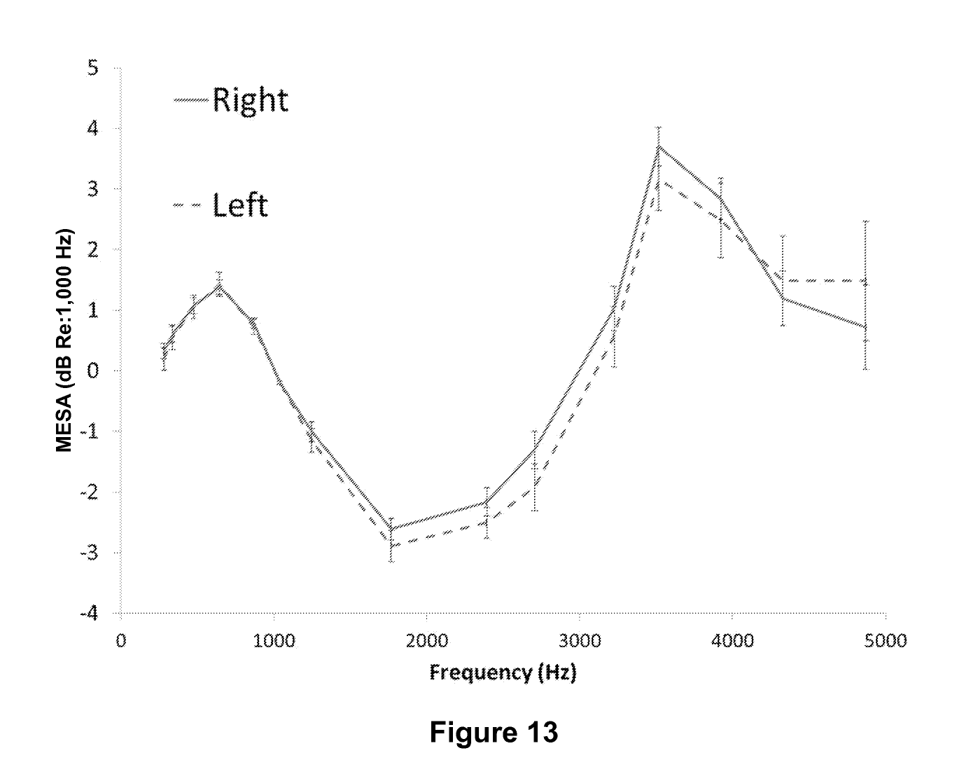

Laterality differences: Consistent with published reports of energy reflectance derived from power flow in the ear canal (Allen, 2005; Beers, 2010), no difference in energy reflectance between the right and left ears is observed (FIG. 13). Greater absorption above 1000 Hz is advantageous to speech perception. Negative values indicate greater absorption of those frequencies compared to absorption at 1000 Hz. A repeated measures ANOVA, with measurement ear as a within-subjects factor, is used to test for laterality in the MESA measure. Due to the heteroscedasticity of the measure, there is a violation of sphericity in the ANOVA (Mauchly's test, X² (104, N=17)=333.89, p<0.001), similar to the EqL analysis. The degrees of freedom are corrected by the Greenhouse-Geisser (ε=0.15) estimate. There was no main effect for ear, F(1,16)=0.34, p=0.57. The model indicated no significant Ear x Frequency interaction, F(2.161,34.6)=1.31, p=0.28.

Gender differences: A repeated measures ANOVA, with gender as a between-subjects factor, is used to test for gender differences in both the right and left ear MESA curves. Since there is no main effect for ear found in the laterality analysis, each ear is tested separately for a gender effect. As for the EqL measure, there is no main effect for gender and no significant Gender x Frequency interaction was found for either the left, F(1,15)=0.54, p=0.48, or right ears, F(1,15)=2.42, p=0.14.

Descriptive statistics: Most parameters are normal in their between-subject distributions. However, several of the distributions are kurtotic, as can be seen in Table 4. The proposed filtering mechanism of the MEMs is broadband in action (i.e., increasing energy absorption above 1000 Hz and decreasing absorption below 1000 Hz). Sensorineural hearing loss, nonlinear interaction with cochlear filtering mechanisms, and other individual differences in sensitivity within the small region of the cochlea stimulated by the pure tone stimulus all suggest a great deal of variance in sensitivity to an individual tone due to non-middle ear structures.

Summary statistics: As described, summary statistics are calculated for EqL and MESA by averaging values in two regions: below 1000 Hz and from 1000 to 4500 Hz. The MESA value at 1040 Hz was used in interpolating the gain at 1000 Hz, for normalization, but was not included in either average. Consistent with the laterality effects on the full measures of EqL and MESA, there is no laterality differences for any of the summary statistics. There is a significant difference in one summary statistic, the difference between the mean EqL level in the mid-frequency and low-frequency ranges in the right ear only, F(1,16)=9.30, p=0.008. However, there are no significant relationships between this statistic and either the MESA, questionnaire, or NiN measures. There is no need to correct for this effect in the summary analyses.

Descriptive statistics: As reported, summary statistics are generated based on the theorized broadband effect of MEM tone on energy absorption and reflectance. A beneficial side effect of this transformation is that the difference measures (e.g., Mean (mid-frequency EqL)−Mean (low-frequency EqL)) are normally distributed in this sample. The mid-frequency range for the EqL measure is from 1250 to 4000 Hz. The low-frequency region of EqL is from 31.5 to 630 Hz. For the MESA measure, the mid-frequency region is from 1248 to 4328 Hz, and the low-frequency region is from 280 to 868 Hz. There is no High-Mid value for the MESA measure because there is only one frequency higher than the mid-frequency bandwidth (Table 5).

Covariation of Numbers in Noise performance and Middle Ear Sound Absorption. Since MESA is a reliable measure of individual differences in MEM energy reflection, the interaction between MESA and NiN_{—}50 is examined. Variance in MEM tone within the sample influences the transfer function of the ME, with increased stiffness causing greater energy absorption in the frequency range of the higher formants (i.e., 1250 to 4500 Hz) and increasing noise tolerance on the NiN task. By reducing energy transmission to the cochlea and decreasing the intensity of frequencies below 1000 Hz relative to higher frequencies within the cochlea, the signal to noise ratio within the cochlea should be increased.

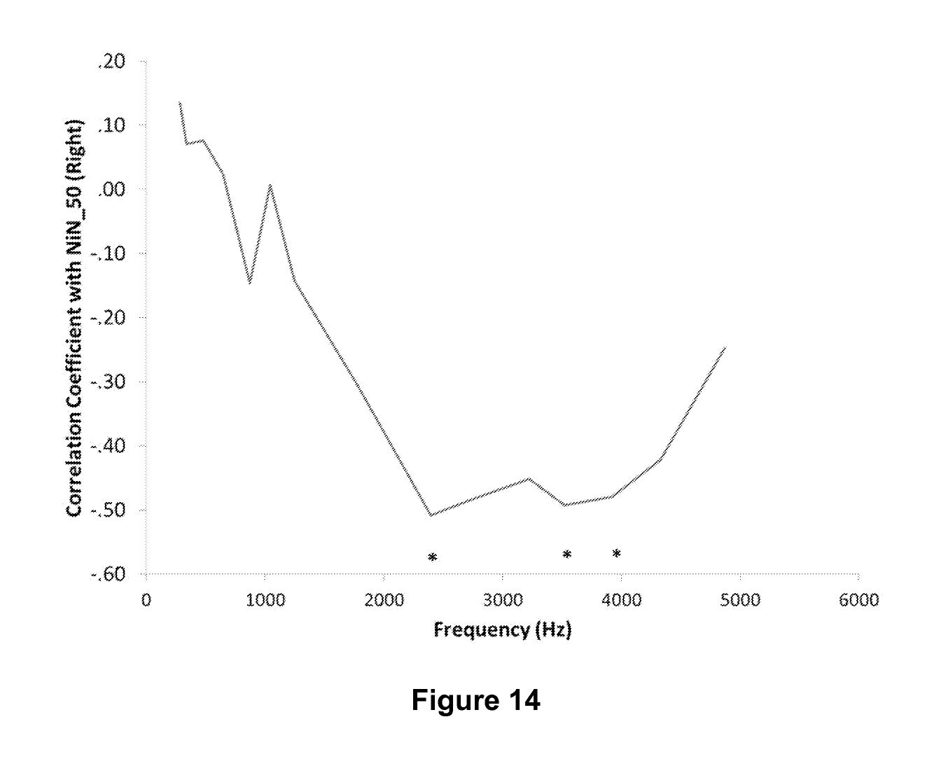

Correlation with individual frequencies of Middle Ear Sound Absorption: The right ear showed a consistent pattern of negative correlations within the frequency band of the higher formants in human speech. Subjects with the highest tolerance for noise reflected less energy in the middle ear (i.e., absorbed more energy into the inner ear) at these frequencies, relative to 1000 Hz (FIG. 14).

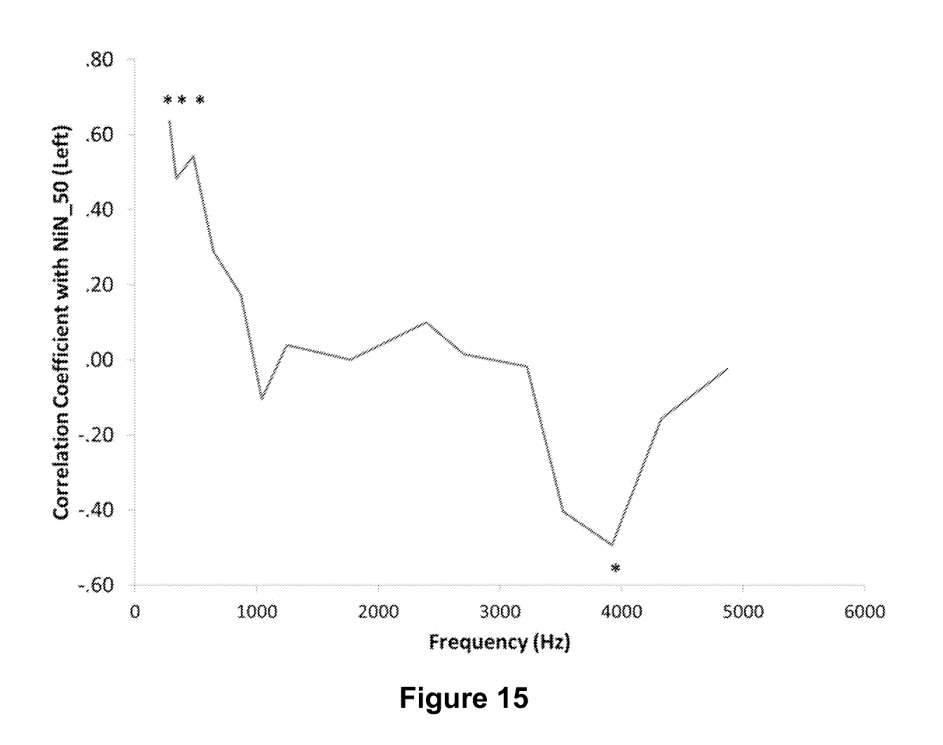

The left ear presents a profile (FIG. 15) not entirely explained by the proposed role of middle ear muscle tone. A strong relationship is found between energy reflectance at the lowest frequencies and noise tolerance. Subjects who absorb less low-frequency energy in the left ear have a higher tolerance for noise. There is also a strong correlation in the expected direction at 4000 Hz. However, there is no relationship between NiN_{—}50 in the left ear and energy reflection between 1000 and 3000 Hz.

Correlation with summary statistics: The correlation between NiN_{—}50 and the MESA summary statistics is examined (Table 6). Energy reflection at a single frequency, rather than over a range of frequencies, is a less precise measure of MEM tone due to individual differences in ear morphology. The proposed role of individual differences in MEM tone is that greater resting tone equates to a wider and deeper region of increased energy transmission to the cochlea from 1000 to 4500 Hz (i.e., the frequency band of the higher formants in human vocal communication). Greater absorption (i.e., less energy reflectance) should correlate with improved performance on the NiN task within this range. By providing the cochlea with more information in this frequency range, relative to the low-frequency masking noise, the task of identifying the spoken number should be facilitated. In essence, there should be a greater signal to noise ratio in the cochlea of individuals with higher neural tone to the MEMs. Below 1000 Hz, energy reflection relative to 1000 Hz should not change as a result of variance in MEM tone. The difference between the mid and low-frequency values is included as the most general measure of middle ear efficiency (i.e., combining the region thought to be due to MEM tone and the ‘passive’ transmission efficiency).

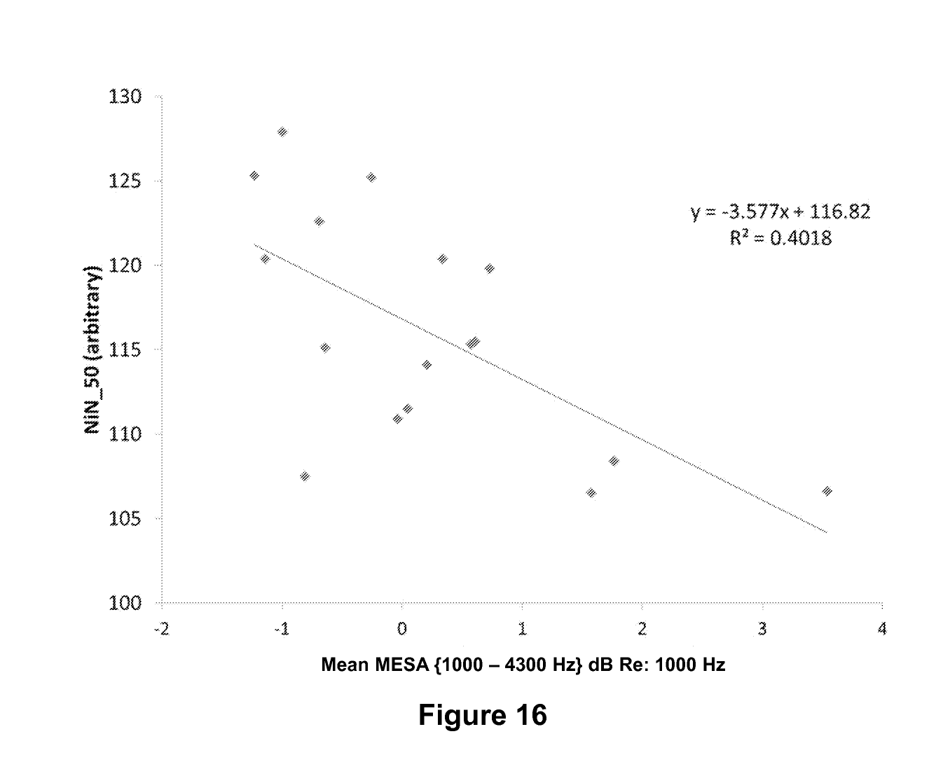

FIG. 16 is the correlation between energy reflectance in the mid-frequency bandwidth and NiN_{—}50 in the right ear illustrated as a scatter plot and left ear (Table 7).

FIG. 17 is the correlation between energy reflectance in the low-frequency bandwidth, below 1000 Hz, and NiN_{—}50 in the left ear as a scatter plot.

Significant effect of Numbers in Noise performance on the MESA curves: To test for the interaction between speech intelligibility (i.e., NiN_{—}50) and energy reflectance, an ANCOVA for the MESA distributions, with NiN_{—}50 as a covariate is applied. Each ear is tested separately, and the main effect of NiN_{—}50 from the ipsilateral ear was significant within the frequency bands used for the summary variables. The significant effects are summarized in Table 8.

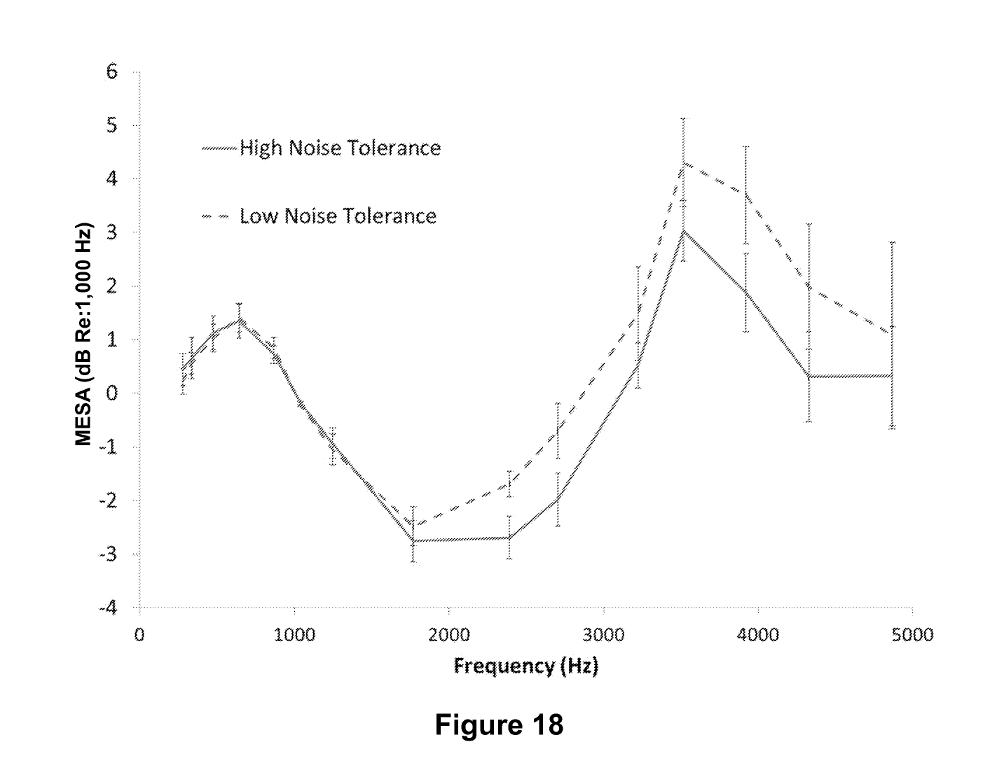

Split-half analysis of MESA as a function of Numbers in Noise performance in the Right Ear: Based on the ANCOVA findings and the significant correlations between the summary statistics and NiN_{—}50, the split-half relationship of MESA in each ear is explored. The normal hearing sample is divided into high and low noise tolerance groups. There are eight subjects in the high tolerance group and nine in the low tolerance group. Visual inspection of the curves for the two groups (FIG. 18) clearly shows that in the right ear the MESA profiles match the predicted difference in energy transfer due to resting MEM tone. Simply, the frequency region of advantage, the region associated with the higher formants of human vocal communication (i.e., 1000 to 4500 Hz) is larger and the advantage greater for the high noise tolerance group. Subjects with reduced tolerance for noise have a narrower and shallower MESAS profile in the region of the higher formants of speech.

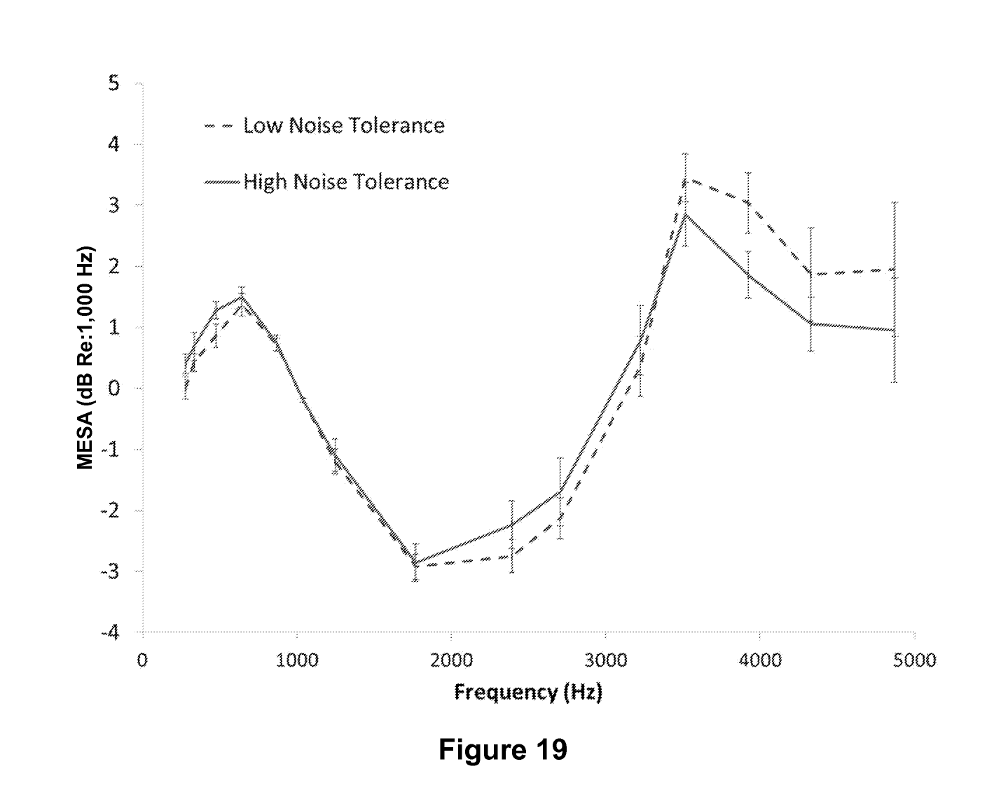

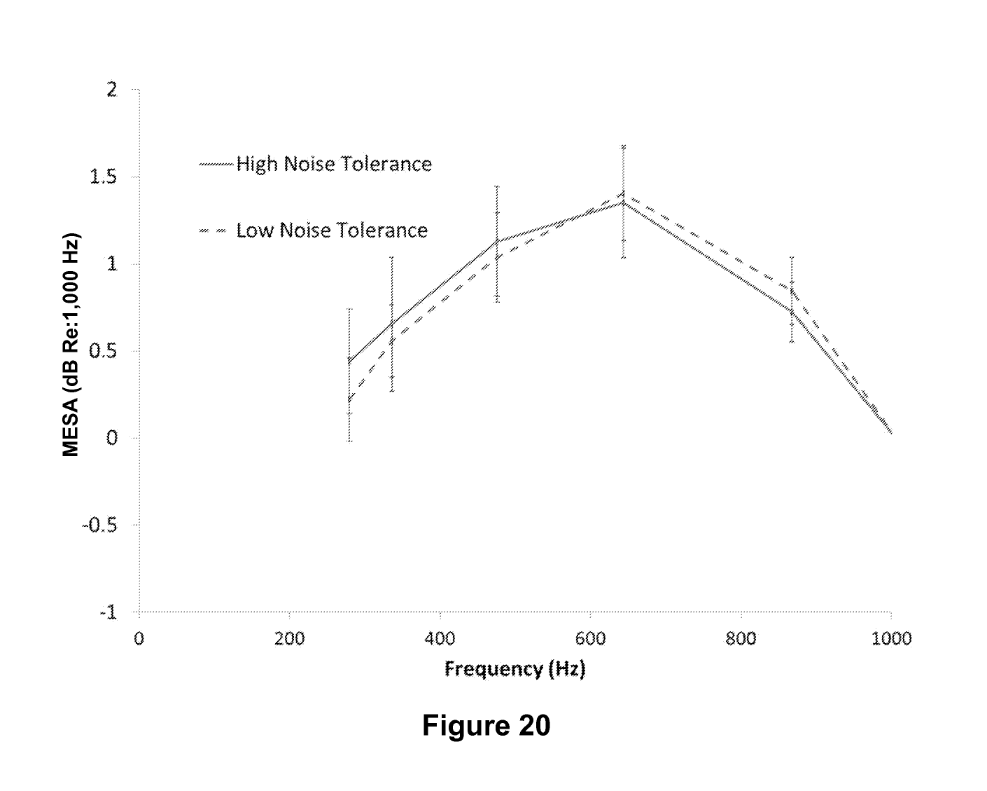

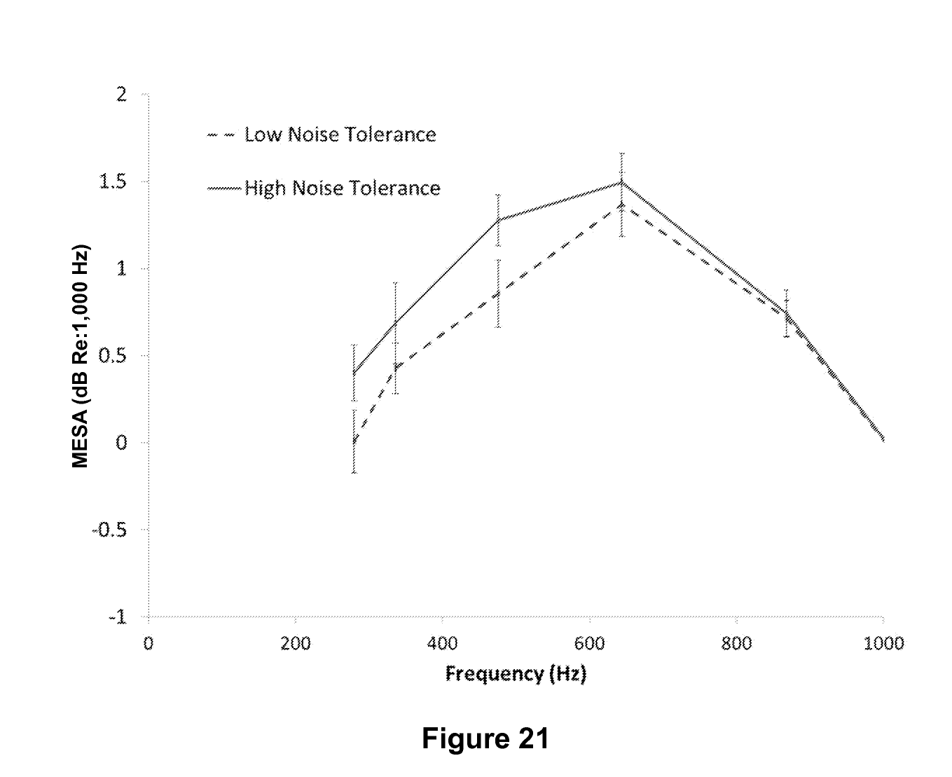

The left ear profile does not complement the model of MEM tone affecting middle ear reflectance (FIG. 19). The shape of the curve from its minimum around 1700 Hz to its next peak near 3500 Hz is not related to NiN_{—}50. The visualization suggests that it is the ability to reflect low-frequency energy and absorb frequencies above 3500 Hz that is related to speech intelligibility in the left ear system. For reference, FIGS. 20-21 are expanded views of the low-frequency limb of the MESA curve for each ear.

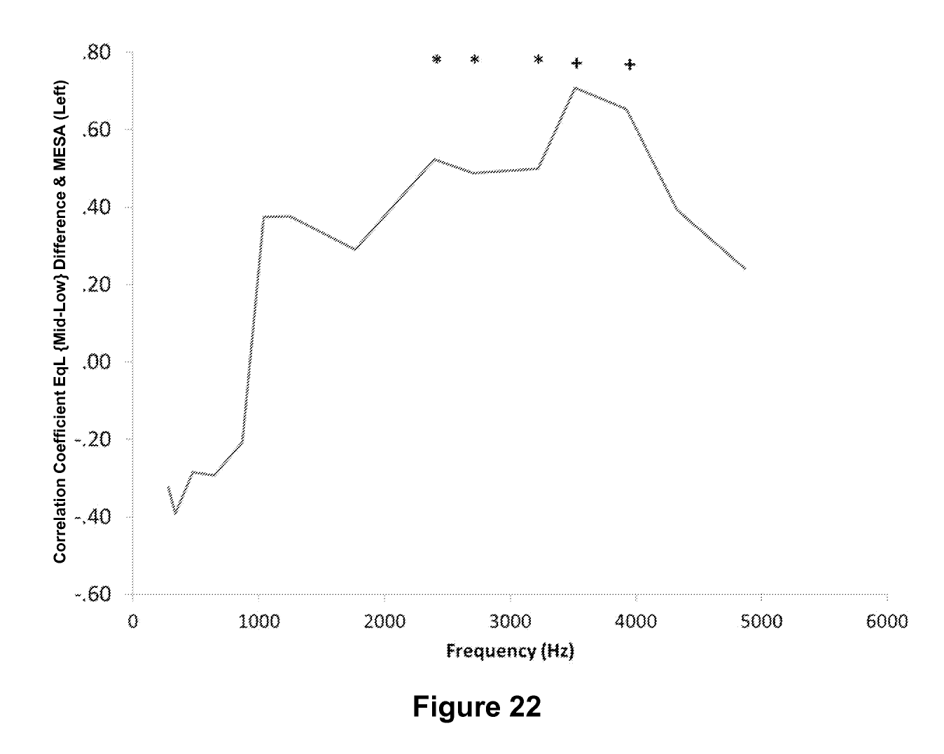

Covariation of loudness perception and MESA: There is no significant correlation between the summary statistics for EqL and MESA in the right ear. However, several strong relationships between loudness scaling (i.e., difference between EqL frequency bands) and energy reflectance in the left ear (FIG. 22) are observed.

Correlation with individual frequencies of MESA: The left ear presents a profile similar to the right ear relationship with NiN_{—}50. A strong relationship is found between energy reflectance in the frequency band of the higher formants of speech and loudness scaling. Subjects who absorb less mid-frequency energy in the left ear increase the intensity of the low-frequency tones on the EqL task less than subjects who absorb more mid-frequency energy. A flatter profile on the EqL task (i.e., smaller difference between mid and low-frequency judgments) is hypothesized to be a feature of hyperacusis. This type of profile is advantageous for detecting a threat (i.e., predator) in the wild.

Correlation with summary statistics: The relationship is quite strong, and clearly driven by the energy reflection in the mid-frequency range (Table 9). This suggests that the frequency band in the left ear that determines speech intelligibility (i.e., low frequencies) may be orthogonal to the frequency band that determines loudness perception (i.e., mid frequencies). It appears that individual differences in MEM tone correlate with loudness perception in the left ear.

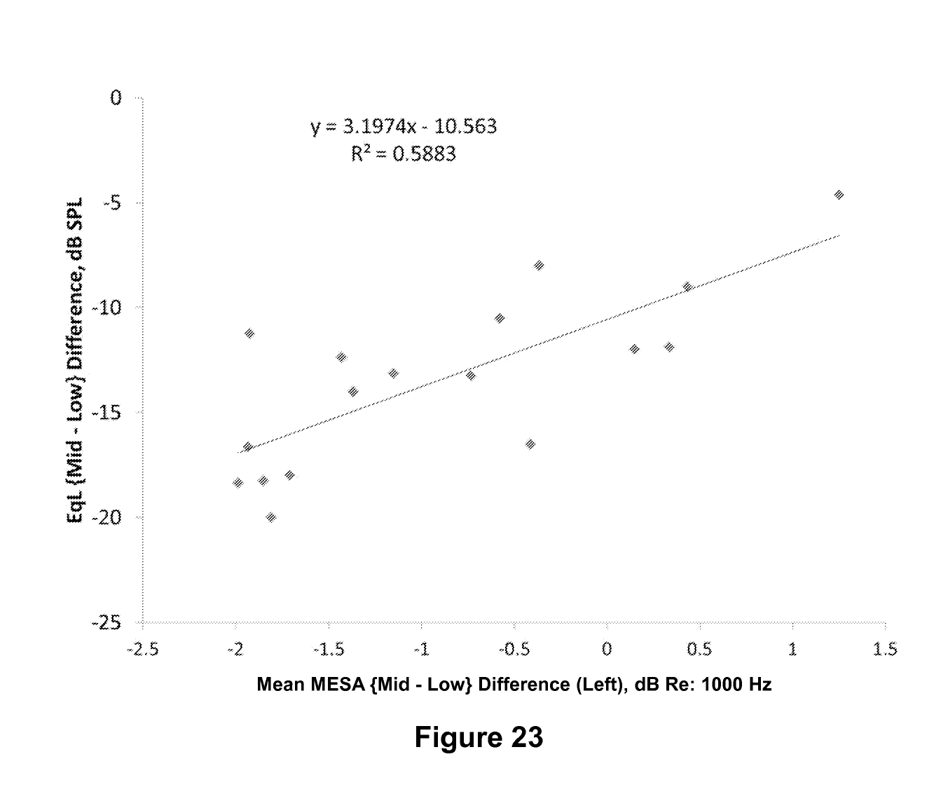

For reference, the relationship between loudness scaling and MESA scaling (i.e., the difference between the average values of each mid-frequency and low-frequency range) is plotted in FIG. 23.

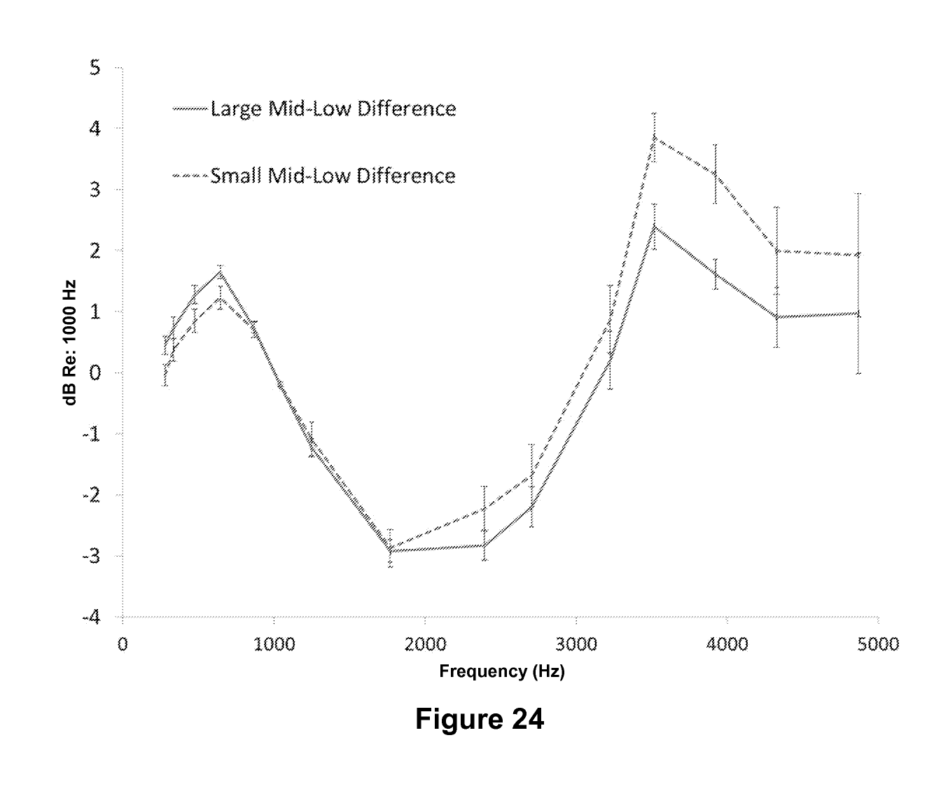

Split-half analysis of MESA as a function of Equal Loudness contour Mid-Low difference in the Left Ear: Following up on the significant correlations, for the left ear only, between EqL judgments and energy reflectance above 2000 Hz, the split-half distributions of MESA as a function of EqL Mid-Low difference is plotted (FIG. 24). Subjects are split again into groups of nine and eight. In the previous split-half grouping the smaller sample was taken from those with high tolerance for noise, assumed to be an indicator of good MEM function. In this case, the smaller sample is from those with the largest mean difference between mid-frequency and low-frequency responses to the EqL task. This is also presumed to be a feature of optimal MEM function. The response matches the hypothesized function of increased MEM tone being associated with greater separation between low-frequency and mid-frequency loudness. In other words, subjects with a smaller perceived difference between high and low frequency pure tones have a narrower MESAS profile, indicating reduced resting tension in the middle ear muscles. The curve closely matches the findings from the right ear, with the groups split based on the NiN_{—}50 score.

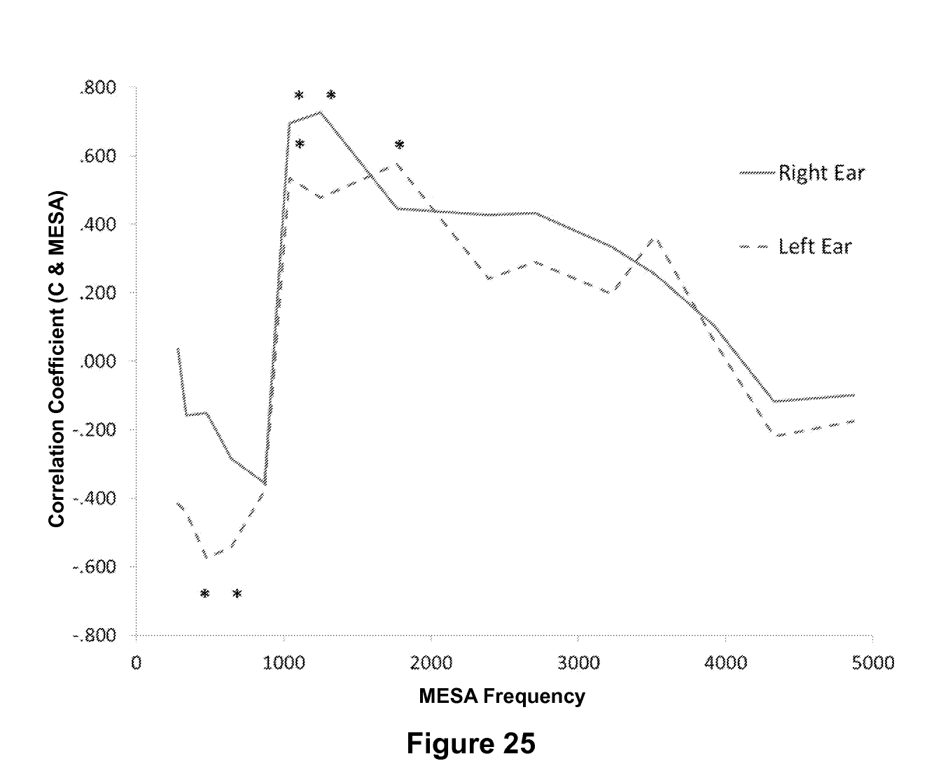

Covariation of MESA and self-reported hearing sensitivity: The composite hyperacusis score also relates to the reflectance measure. This relationship is not uniform across frequency, similar to the relationship between NiN_{—}50 and MESA. When referring to FIG. 25, it is important to remember that the measures are normalized at 1000 Hz, so the directionality of the correlations is expected to change at this point. Energy reflection in the left ear is significantly related to self-reported sensitivity from low frequencies through 2000 Hz, while the right ear showed a narrower band of significant correlations close to 1000 Hz. Both ears showed a similar profile of correlations above 1000 Hz, suggesting that a weighted average of MESA may provide a more reliable estimate of hearing sensitivity than any one frequency.

Study 2: Treatment Effect: One Week of the Listening Project: This subject is an adult male with a diagnosis of autism spectrum disorder. The subject possesses developed verbal skills and presented as a reserved but friendly individual. The subject was very interested in the computer based assessments, although the subject did fixate on several trials in the EqL task. This perseveration on the computer based tasks led to a difficult testing session with all of the participating researchers agreeing that his responses were not valid. In essence, the subject enjoyed manipulating the intensity of the tones in the EqL task, but did not appear to make any decision regarding the loudness matching portion of the task. He simply adjusted each tone until bored then moved on to the next trial.

Although unable to obtain reliable measures of listening performance from this subject, we were able to obtain reliable MESA measurements at each session. The main finding from this subject is flexibility in the MESA measurement, with one ear measurement changing while the other stays the same. This change is proposed to be a result of the auditory intervention, The Listening Project. The findings are replicated during a follow-up study performed two months after the initial auditory intervention. On this follow-up visit the subject repeated pre and post testing and received the full day 5 intervention audio between the pre and post measures.

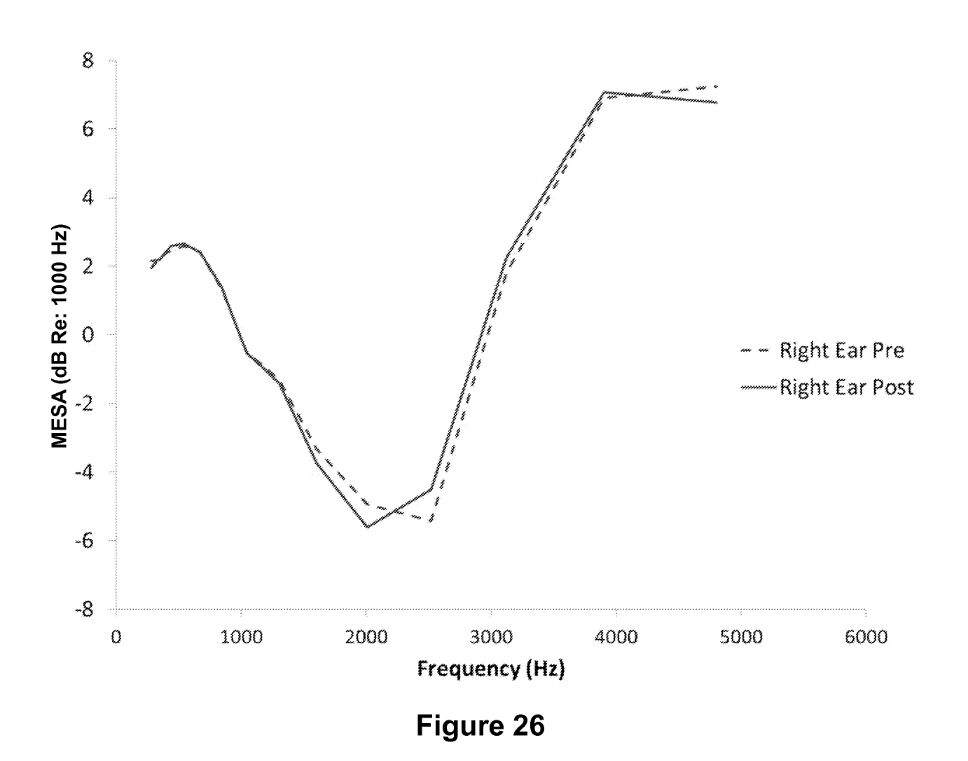

New probe tips are used on each visit to the lab: one for pretesting, one for post, and one for the full follow-up visit. FIG. 26 is the test-retest reliability in the right ear during the follow-up visit.

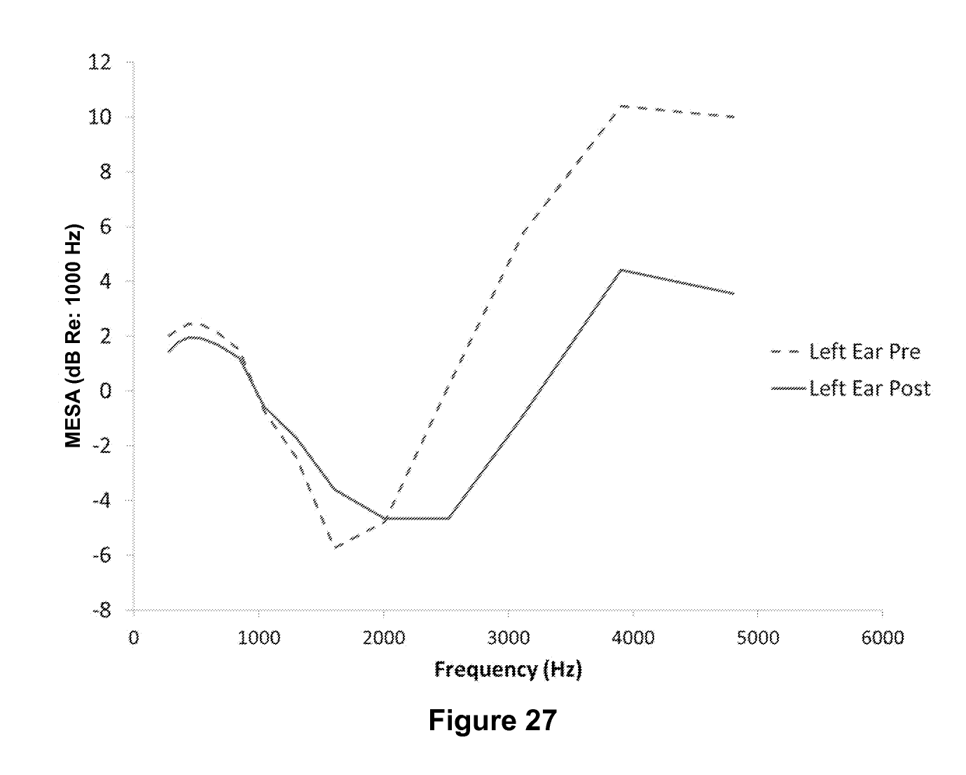

Over the course of the intervention, the subject became increasingly comfortable with the staff and research personnel. On arriving for post-testing of MESA, the subject indicated that he had listened to the same music he had been listening to on Monday. He indicated that on Friday it was easier to understand the words. Over the course of that same week, this change in MESA is observed within the left ear (FIG. 27).

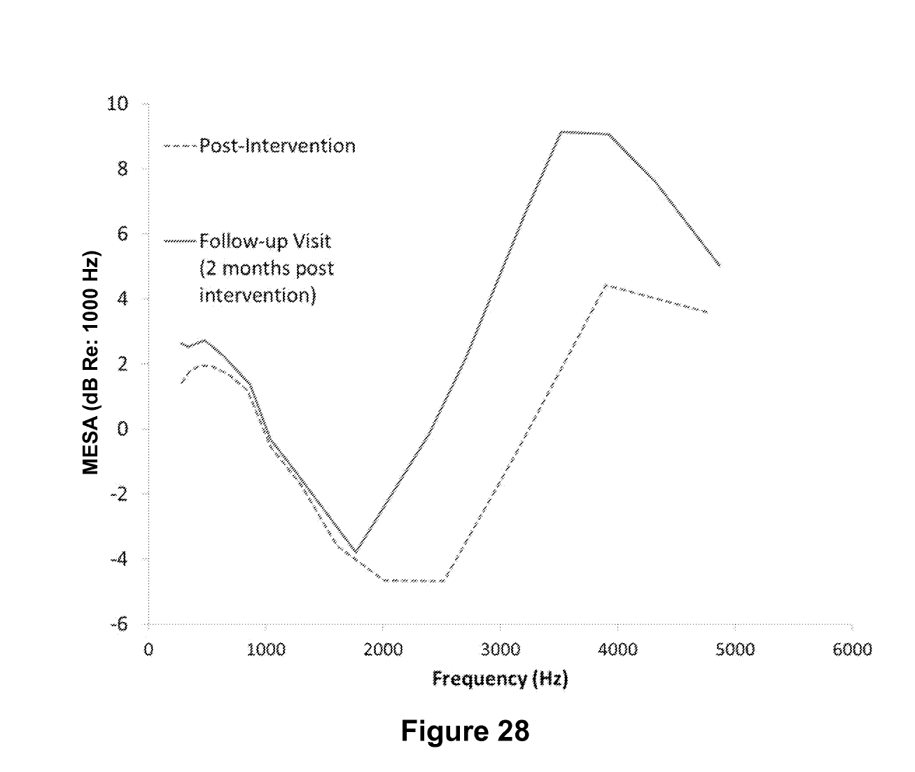

On returning to the lab two months after receiving the intervention, the subject reports several improvements in social engagement behaviors. He had taken on a job in a noisy environment, one which he previously attempted but could not tolerate. While he was friendly at the first meeting, he was noticeably more outgoing during the follow-up visit. However, in his left ear his MESA profile had reverted to the shape seen on the first day of testing (i.e., before the intervention) (FIG. 28).

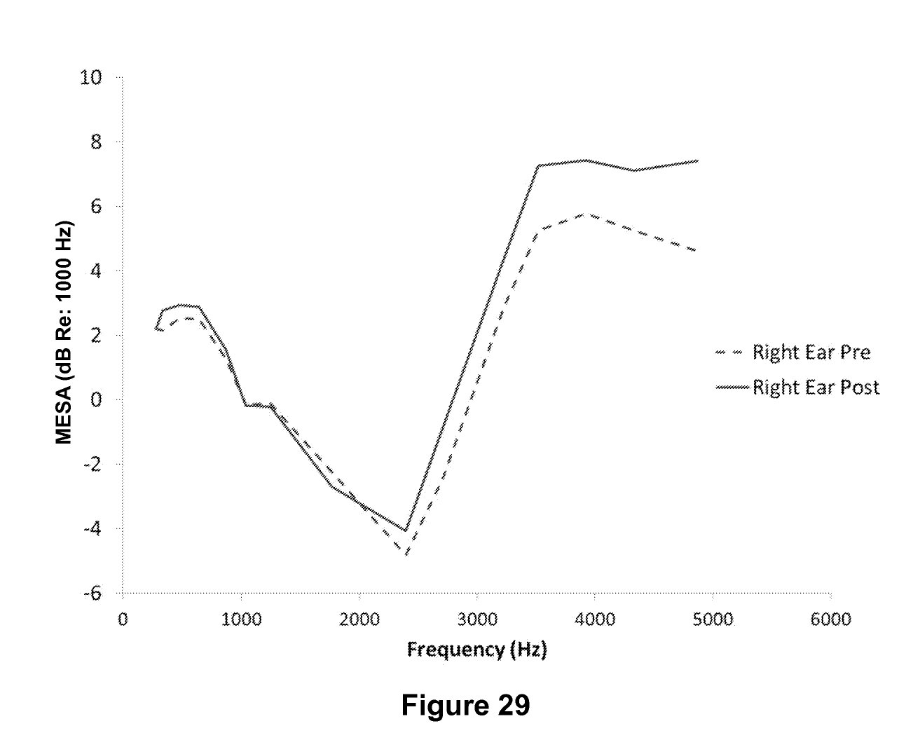

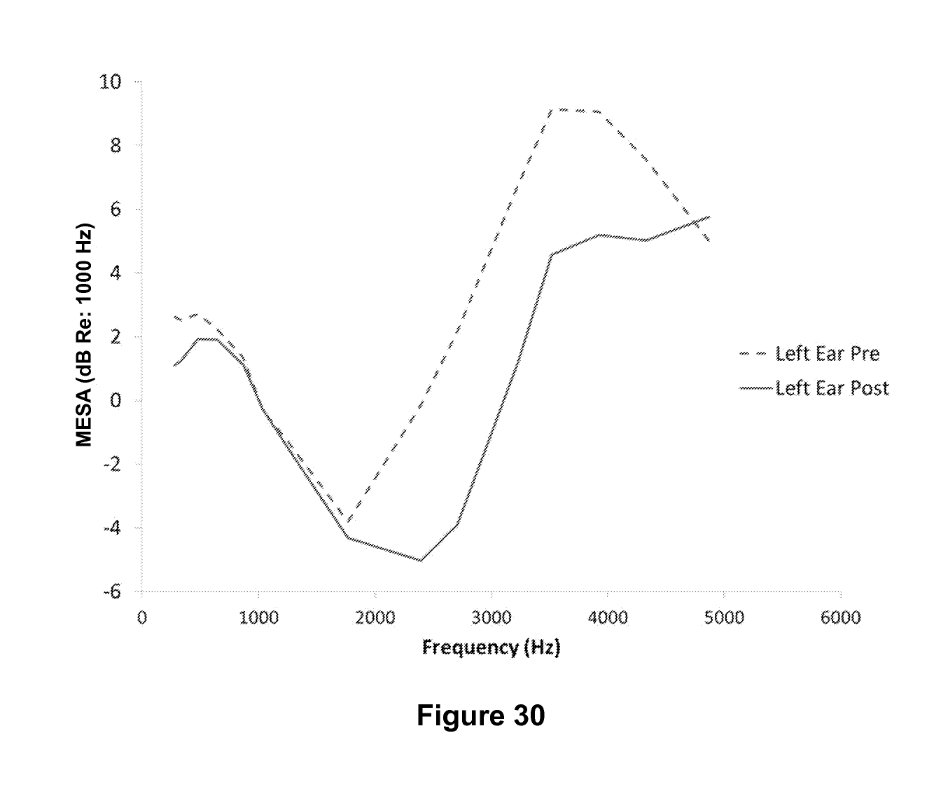

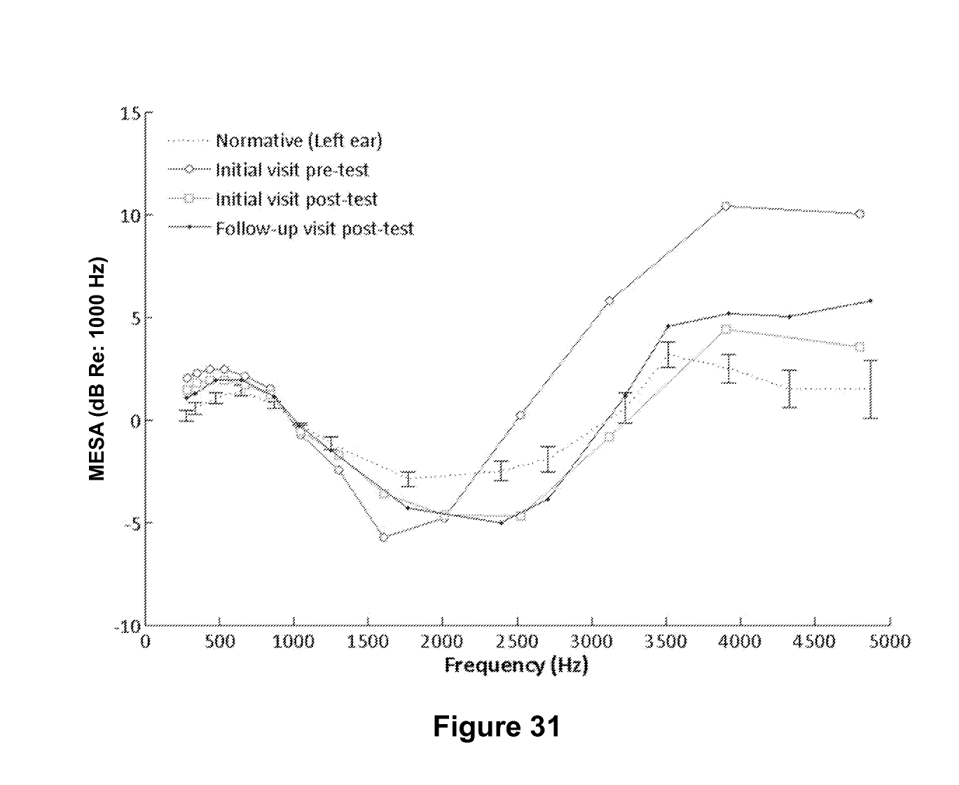

He repeated the cognitive testing and then listened to the full Listening Project intervention from day 5. After a break of about five minutes, he returned to the lab and repeated his MESA measurements. As before, the right ear showed only minimal change (FIG. 29). However, his left ear showed significant change in energy reflectance (FIG. 30), similar to the changes after the first round of the intervention.

Qualitatively, the left ear transitions from having a smaller than normal frequency band of increased energy absorption in the middle ear to having a wider and deeper than normal region of advantage (FIG. 31). The frequencies used in the probe at the follow-up visit were the same as the normative data from study 1. Based on this normal hearing sample, the subject had a left ear MESA Mid-Low difference with a z-score of 9.39 at pretesting. This changed to −1.72 at the posttest measurement, within the middle 95% of the distribution. This is the first demonstration of physiological changes in the middle ear as a result of an auditory intervention.

The primary findings demonstrate that individual differences in middle ear reflection within a normal hearing population, along the dimensions consistent with MEM tone, are related to loudness scaling in the left ear and speech intelligibility in the right. Thus, the neural regulation of the resting tone of the middle ear muscles is functionally adjusting the “gain” of the auditory system along a continuum from hypersensitivity to low-frequency noise with poor speech intelligibility at one end to normal sensitivity with good speech intelligibility (but less vigilance to external threat) at the other end (Porges & Lewis, 2010). The gain of the middle ear is greatest around the resonant frequency, approximately 1000 Hz, but the roll-off on each side of this frequency is modulated by resting tension applied by the middle ear muscles.

Within the healthy population, the functional impact of small variations in right and left middle ear muscle tone were observed by measuring loudness perception and speech intelligibility independently in each ear. The findings have broad clinical applications since hearing sensitivity and difficulty in extracting the information from voice in background noise have been reported as a feature of several psychiatric disorders (Stansfeld, 1992). Based on the findings herein, those reports can be reasonably explained by disruption in the neural regulation of the middle ear muscles on both sides of the head.

The decision to employ monaural measurements of loudness perception, speech intelligibility, and energy reflectance is validated by several significantly different relationships between right ear and left ear measures. The reflectance measure is novel and the findings from the current study provide insights into an overlooked filtering mechanism in the auditory periphery, resting tone of the middle ear muscles. The laterality of middle ear function is consistent with the laterality of the vagal regulation of the heart and the neural regulation of the striated muscles of the face (see Porges et al., 1994).

This consistency between regulation of muscles of the face and vagal regulation of the heart provides the possibility that resting middle ear tone may be part of the same neural feedback circuit that links dynamic autonomic nervous activity to expressed changes in the neural regulation of the striated muscles of the face and head controlling facial expression and vocal prosody. Thus, the ability to listen either to human voice or the low frequencies associated with predators would be dynamically coordinated with both physiological (i.e., calm to listen to human voice or activated to fight or flee) and the emotional expressions of the face and vocal intonation of voice. This supports the model of a social engagement system that adjusts special visceral efferent output based on detected social cues in the environment (Porges, 2007; Porges & Lewis, 2010). In support of this model receptive language skills, vagal regulation of the heart, and engagement behaviors (i.e., orienting towards the speaker) have recently been shown to covary within a population of children with ASD. Children with greater vagal inhibition of heart rate while being spoken to have better language and communication skills later in life (Watson, 2010).

The middle ear ossicles are regulated by two middle ear muscles. Although the literature focuses on the stapedius muscle, regulated by a branch of the facial nerve, the tensor tympani is also involved in the regulation of middle ear structures via a branch of the trigeminal nerve. Reflexive contraction of the stapedius muscle in response to intense acoustic stimulation is bilateral, as is the reflexive contraction of the tensor tympani to internal behaviors like chewing and vocalizing. This does not preclude the possibility that the tension applied by these muscles in a quiescent state could vary between the right and left sides. In the current study, a relatively homogeneous group of “normal” young adults were tested. It is assumed that with a larger, more heterogeneous population, the range of differences in the variables (sensitivity, loudness perception, speech intelligibility and energy reflectance) will increase. Similarly, it is also assumed that as clinical populations are tested, the defining features of pathology will be identified, including the parameters of dysregulation of the middle ear muscles that may or may not be bilateral.

The autonomic regulation of the resting MEM tone may be lateralized, as the autonomic system is in general (Porges, Roosevelt, Maiti, 1994). This difference in neuromuscular tone may represent an individual difference that is constant (e.g., greater density of neural connection from one hemisphere) or dynamic (e.g., a balance adjusted depending on context). In either case, within this restricted sample (young and healthy with good hearing), these laterality differences can be used to examine the functional role of MEM tone on hearing in each ear. Research on individuals with concomitant hyperacusis and difficulty hearing in noise can explore the lateral differences in MEM tone. Some disorders associated with autonomic dysregulation will impact the resting tone of the muscles in both ears, while other conditions (or possibly other individuals with the same diagnosis) will show impairment in only one ear, such as seen in the case study of the Listening Project intervention.

Consistent with these findings, click evoked brainstem auditory evoked potentials have reliably been reported to indicate greater amplitude responses in the right ear (Levine, 1983; Levine, 1988). More recent work on the brainstem level encoding of acoustic information has indicated lateral differences specific to speech-like information at the level of the thalamus (King, 1999) and the brainstem (Abrams, 2006). These laterality differences for language comprehension are largely independent of handedness, as they arise from the efficiency of neural communication between the ears and cortical structures that process language and these structures are consistently lateralized regardless of handedness. Nevertheless, the potential for handedness to interact with regulation of resting middle ear muscle tone should be explored in future research using MESA.

Left ear measures are sensitive to features of hyperacusis: There is evidence of a significant laterality in noise induced sensorineural hearing loss, with the left ear more likely to suffer both permanent (Nageris, 2007; Boger, 2009) and temporary (Pirila, 1991a; Pirila, 1991b) threshold shifts in response to noise exposure at damaging intensity. However, reports of acoustic power flow in the ear indicate no right/left differences in tympanometry (Feeney, 2004), impedance (Allen, 2005), or reflectance (Beers, 2010) at the eardrum, which is in agreement with the MESA measure. It is plausible that the increased prevalence of noise induced hearing loss reflects a laterality difference in the transduction mechanisms of the cochlea. The reported relationships between perceived loudness (EqL) and left ear reflection suggests that loudness growth in the low-frequency end of the left ear system is related to middle ear muscle tone.

This does not hold true for the right ear. The transduction mechanism that maintains this loudness scaling (from the middle ear to the final perception of loudness) is possibly also responsible for the increased susceptibility to noise induced damage within the cochlea. Findings reported herein suggest laterality in the processing of speech in noise, since the ears are sensitive to different portions of the frequency spectrum with regards to this task. This adds to the body of evidence that identical information received at the cochlea is not compressed into auditory nerve firings in the same manner (Hornickel, 2009).

In addition to the left ear relationship between loudness perception and low-frequency reflection in the region of 1250 to 4500 Hz, a relationship between left ear reflection of low-frequency energy and self-reported sensitivity to noise (i.e., the hyperacusis composite score) is reported. Those differences are not likely due to variance in middle ear muscle tone, since the middle ear muscles should have a flat attenuation of absorption for frequencies below 1000 Hz (Pang, 1989). However, there is the possibility of an interaction between middle ear muscle tone and the resonance of the ear canal that accounts for this difference. In addition, the observed difference in the lowest frequencies may represent structural abnormalities in the middle ear (e.g., perforated ear drum). This can be explored through existing instruments (i.e., tympanometry, acoustic impedance), and, can be examined in studies that incorporate those more traditional clinical devices with the MESA measurement.

The left ear system is theorized to have a more direct connection to the neuroceptive circuits of the right vagus (see Porges et al. 1994). Darwin observed that monkeys communicate “anger and impatience by low” tones. Environmental dangers are also associated with low-frequency noise (e.g., earthquakes, approaching footsteps). All of these signals trigger reflexive responses to flee the source of the noise. Perhaps a left ear system characterized by over absorption of low-frequency energy biases the individual to hyperarousal or increased vigilance to the surroundings. This explains the strong left ear relationship between energy reflection and reported sensitivity to environmental noise.

Right ear measures are related to speech intelligibility: The predicted relationship between MESA and speech intelligibility in the right ear is found. The right ear auditory system may be more sensitive to the relative change in energy transmission within the mid-frequency range than that of the left ear system. This is likely due to the compression of acoustic information as it travels from the cochlea to the language processing centers, maintaining information that was amplified by MEM tone that the left ear system discards. There is an established right ear advantage for processing language in binaural stimulation (Hugdahl, 2001). This is possibly a function of more direct neural connection with fewer synapses between the right ear auditory nerve and the language processing centers of the brain. The compression of information as it travels along this pathway shows speech specific encoding differences between right and left ear information at the level of the brainstem (Hornickel, 2009). These effects include increased spectral resolution in the region of the first and higher formants for the right ear as well as decreased latency to brainstem responses for right ear speech. A right ear system that has become specialized for processing complex language stimuli may maintain the flexibility to attend to this information only in safe settings by regulating the middle ear muscles as hypothesized.

The compression of left ear information could complement the right ear system by accurately reflecting the perceived acoustic environment with respect to the spectral envelope. The compression of information in the left ear would then reliably convey the amount of low, mid, and high frequency energy received by the cochlea while the right ear system would sacrifice this intensity information in exchange for greater fidelity in the pitch differences at the mid frequencies (as transduced by the cochlea).

Regardless of the mechanism, there is a difference in signal compression at the cochlear level for nonspeech information (Sininger, 2004) and the brainstem (Sininger, 2006). This is just the first processing difference in the right ear signal transmission pathway and is also evident in measures from the cortex (Abrams, 2008). The differences continue throughout the processing with the overall transmission path from the right ear to the left hemispheric speech regions being more direct for at least parts of the auditory information (Heffner, 1990).

Clinical application: a neural component to conductive hearing loss. The measurement of middle ear muscle tone described herein, in addition to static middle ear power flow, provides a clinician or researcher with tools to more fully determine the conductive component of any hearing difficulties. The test is quick and reliable, with consistent measurements in both ears being obtained in less than five minutes with most subjects. By using hardware that is already approved for measurements of otoacoustic emissions, in both adults and infants, the measurement can be translated into clinical practice easily. Subjects across the full age and functional range can now have their middle ear status assessed efficiently.

Currently, middle ear power analysis (e.g., Keefe, Margolis, Feeney, etc.) is useful to screen for gross abnormalities in energy transmission due to blockages in the middle ear. Complex tone energy reflection, measured within a bandwidth influenced by middle ear muscle tone, provides information on a potentially critical feedback system within the middle ear. This information is currently ignored as changes in middle ear muscle tone are not considered to occur outside contractions due to acoustic stimuli (i.e., the acoustic stapedial reflex) or internal events like chewing or vocalizing. However, the individual differences reported in each ear provide evidence that this peripheral filter is being tuned, and this tuning is playing a significant role in the comprehension of speech and the perception of loudness.

Any impairment of this feedback system will lead to difficulty modulating the resting tension on the middle ear muscles. One individual may develop spasticity, which would increase the relative amplitude of frequencies above 1000 Hz within the cochlea. Another may have atrophy and decreased stiffness in the ossicle chain regardless of context. Both may present with acceptable audiometric levels due to intact middle ears and healthy cochlea; however, the individual with atrophy will have more difficulty hearing in a noisy environment where the relative amplification of frequencies above 1000 Hz would facilitate speech comprehension.

Further, from a clinical perspective, otoacoustic emissions (0AEs) are rapidly becoming an integral component in the assessment of cochlear function. Variance in MEM tone will influence the reverse transmission along the ossicle chain of this information. Changes in OAE amplitude may represent changes in MEM tone due to social context or another mechanism regulating the resting tone on the MEMs. Consider the situation: A child is referred to the clinic, and on the first visit, distortion product OAEs (DPOAEs) cannot be measured in either ear. On a subsequent visit, the DPOAEs are recorded with normal signal to noise levels. Does this reflect a change in cochlear filtering mechanisms, or could the subject simply be more relaxed during the follow-up visit, with greater tension being applied to the MEMs, and thus, greater amplitude DPOAEs being transmitted through the stiffened ossicle chain?

Clearly, more needs to be determined about the temporal aspects of MEM tone. How does it change over the course of a normal day? The final case study suggests that it is dynamic, but measures from typically hearing individuals are relatively stable (data not shown). Does it change with age? The sample was homogenous and young, but the autonomic nervous system undergoes significant age related changes in almost all aspects.

The most obvious clinical application of the technology is as an objective screening tool for hyperacusis. While the results from this normative sample should be extended to subjects who exceed the clinical threshold for hyperacusis, the correlation between left ear reflectance measures and established self-report measures of hearing sensitivity indicate that MESA is potentially the first objective measure of hyperacusis symptoms. Currently, there is no accepted physiological model that explains the features of hyperacusis. Failure to properly regulate the left ear muscle tensions is a mechanism for hyperacusis that has not fully been explored but shows promise. These studies should include traditional measures of ME power flow in order to determine if the effect is structural or neural.

Finally, by further refining the sensitivity of existing measures of the conductive hearing system, the technology brings clinical diagnosis of sensorineural hearing loss forward. The ability to identify pathological middle ear systems, both with structural abnormalities and now with neural regulation deficits, means that clinicians, for example, can exhaustively test and then eliminate any conductive component to detected hearing losses. Thus, the location of the deficit can more reliably be placed in the central hearing structures (e.g., the cochlea, the brainstem).

Research tool: a new measure of physiological state. These results demonstrate the applicability of the device for testing interventions that purport to treat language processing deficits. Particularly, this MESA measurement is designed to provide a fast and reliable measure of individual differences in MEM tone. This is most difficult in clinical populations that include aggressive behaviors and acoustic and/or tactile hypersensitivities. While other measures of acoustic power flow through the middle ear may also be employed, it may not be possible for extremely sensitive individuals to withstand the pulse train of clicks or chimes necessary for those measures. We can establish the transformation between MESA and those measures of acoustic power flow across the full frequency spectrum. Once this relationship is established in normal hearing individuals, it will not be necessary for hypersensitive subjects to participate in both tests. Since the test provided herein does not rely on subject response, the measure of hearing sensitivity is objective.

A rigorous test of auditory interventions based on objective physiological data is desperately needed in the ASD community (as well as other clinical populations). Interventions are varied, costly, and often advertised with bold claims that are not founded in empirical research. The results presented with the Listening Project intervention suggest that such a quantitative test can be derived from energy reflectance in the ear canal.

The normative data also support the theoretical model on which the Listening Project was developed. Even within the restricted range of individual differences (none of the subjects exceeded Khalfa's hyperacusis threshold of 26) there was a strong relationship between low-frequency energy reflection in the left ear and the composite hyperacusis score. Left ear reflectance in the mid-frequency band was also related to the EqL profile, with “low MEM tone” individuals having a flatter EqL profile. This is a unique contribution to the understanding of the interaction between psychophysical perceptions, physiological state, and hearing sensitivity. The Listening Project was designed around a theoretical model that physiological state modulates both sensitivity to noise and the perception of loudness through regulation of the middle ear muscles.

The right ear shows a relationship between mid-frequency energy reflectance (hypothesized to be under the influence of MEM tone) and speech intelligibility. This finding suggests that individuals with chronically heightened vigilance (i.e., increased sympathetic activation) may be at a disadvantage for understanding human voice. Additionally, emotional information is conveyed through the higher formants of human speech (i.e., in the frequency range above 1000 Hz). Therefore, a deficit in speech intelligibility due to the middle ear transfer function should reduce emotional intelligibility as well. Thus, the findings suggest a potential link between MEM tone, physiological state regulation, language development, and vocal affect comprehension.

Another dimension of variance not addressed by the current study is the relative contribution of the two middle ear muscles to the energy reflectance of the middle ear. Since the two muscles (stapedius and tensor tympani) differ considerably in size and temporal characteristics of their responses (Borg 1972a; Borg, 1973), their contributions to middle ear tension should be different at steady-state and during dynamic changes. Research involving unique cases with disruptions to specific middle ear muscles will determine the parameters of this relative contribution from each muscle.

Provided herein is a novel measurement of energy reflected from the activated ear canal. Measurements are optimized to maximize individual differences in energy reflectance from the ear canal due to variance in resting MEM tone. MESA magnitude was differentially related to loudness perception and speech intelligibility in each ear. In the right ear, the hypothesized relationship between increased absorption of frequencies corresponding to the higher formants correlated with improved speech intelligibility. In the left ear, the hypothesized relationship existed between increased loudness differences between high and low tones and energy absorption in the frequency range of the higher formants. These findings have implications for the autonomic regulation of the quiescent state of the middle ear muscles, which may be lateralized in a normal hearing population.

### Example 2

### Atypical Hearing Evaluation

This example investigates covariation between neural regulation of middle ear muscles and functional measure of hearing in a population of normal hearing young adults and atypical subjects. One measure of “hearing” relates to the ability to understand spoken words in the presence of noise. We use measurements from a novel device, referred herein as a “middle ear sound absorption system” (MESA). The MESA device as a number of advantages, including being a fast screening tool, with a reliable trial taking about 10 seconds, and at least two trials are provided per ear. The MESA device and procedure has a high test-retest reliability, including with probe replacement.

As discussed, the device and methods relate to measuring the absorption at the ear drum as a function of frequency, such as be detecting the reflected energy from an acoustic sound-wave input. In an embodiment, the input is a non-harmonic acoustic input comprising a comb input that impacts the middle ear in a manner that is fundamentally different than pure tones or other conventional inputs. In this manner, a frequency-dependent absorption is obtained, with the plot providing the ability to pinpoint potential concerns related to the middle ear. For example, increased resting tension in the middle ear muscles increase absorption at frequencies above about 1250 Hz. Greater absorption of higher frequencies, relative to those about 1000 Hz and below facilitate the “unmasking” of speech in noisy environments. In addition, wider and deeper bowls in the frequency spectrum by the MESAS device is expected between about 1200 and 3500 Hz.

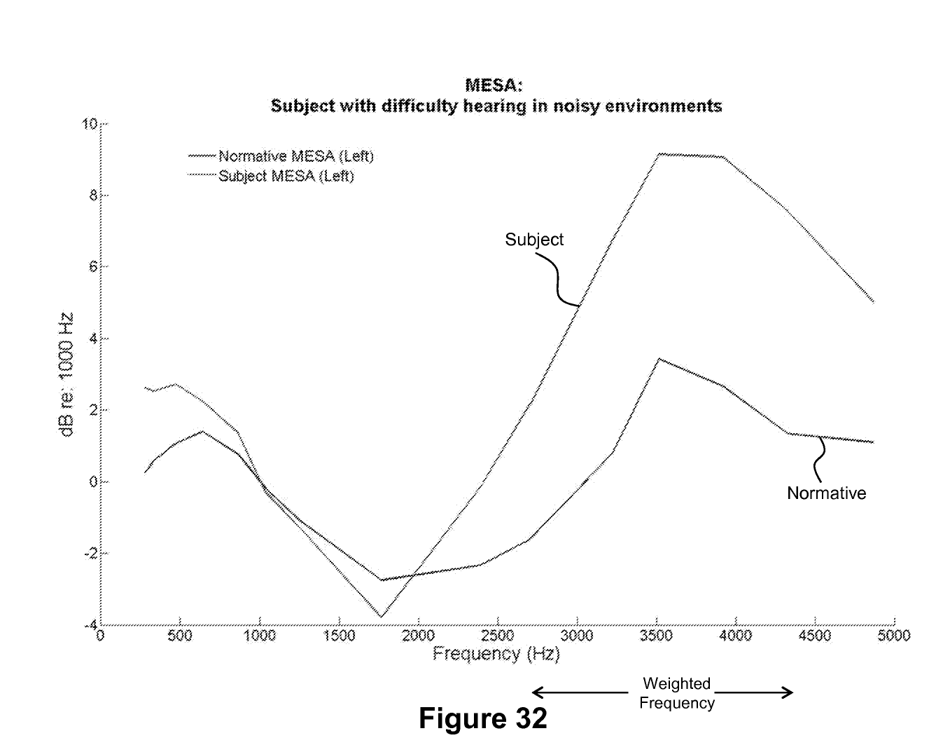

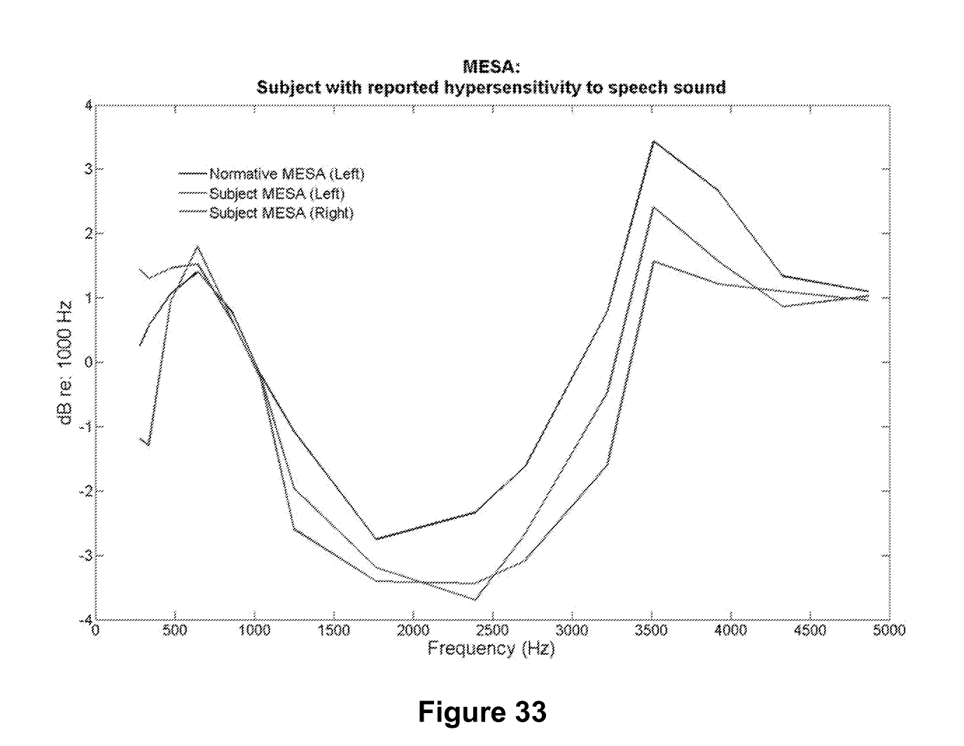

As discussed, the reflected energy has been measured for an autistic subject, along with effect of a therapeutic treatment (see, e.g., FIGS. 26-31) FIG. 32 shows the measured reflected energy for an individual with difficulty in hearing in a noisy environment (labeled “subject”), relative to a reference (labeled “normative”) The upward shift of the spectrum at higher frequencies indicate the test subject has difficulty hearing in noisy environments. FIG. 33 is the reflected energy for a subject with a reported hypersensitivity to speech sound (labeled “subject”) for each of the left and right ear. For comparison and assessment purposes, a reference is provided from a normal or typical individual.

Individuals having difficulty hearing show increased level of reflected energy (e.g., less absorption) over certain frequency range (see FIG. 32). In contrast, an individual with hypersensitivity to sound show showed a decreased level of reflected energy (e.g., greater absorption) over certain frequency range (see FIG. 33). These measures of reflected energy also illustrate the applicability of various algorithms to assist in quantifying and assessing a subject for one or more atypical hearing states or conditions. For example, an algorithm mate be employed to assess difficulty hearing in a noisy environment and another for hypersensitivity to speech sound. The “weighted frequency” label in FIG. 32 represents a region where a frequency may be weighted in an algorithm to assist in assessing subject status. In this manner, differences from a reference can be rapidly calculated and quantified, thereby assisting in assessment in a pass (result is typical)/fail (e.g., likelihood that the measurement is associated with an “atypical” state) that is not subjective.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

Whenever a range is given in the specification, for example, an intensity range, a time range, or a sensitivity range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The definitions provided herein are to clarify their specific use in the context of the invention.

One skilled in the art readily appreciates that the present invention is well adapted to carry out the objects and obtain the ends and advantages mentioned, as well as those inherent in the present invention. The methods, components, materials and dimensions described herein as currently representative of preferred embodiments are provided as examples and are not intended as limitations on the scope of the invention. Changes therein and other uses which are encompassed within the spirit of the invention will occur to those skilled in the art, are included within the scope of the claims.

Although the description herein contains certain specific information and examples, these should not be construed as limiting the scope of the invention, but as merely providing illustrations of some of the embodiments of the invention. Thus, additional embodiments are within the scope of the invention and within the following claims.

### TABLES

TABLE 1
Distribution parameters of the composite hearing sensitivity
score, C.

| | Mean | SD | Skewness | Kurtosis |
|---|---|---|---|---|
| C | 0 | 0.91 | −0.53 | 0.99 |

TABLE 2
Distribution parameters of the noise tolerance variable.

| Variable | Mean | SD | Skewness | Kurtosis |
|---|---|---|---|---|
| NiN_50_Right | 116.06 | 6.935 | 0.143 | −1.177 |
| NiN_50_Left | 114.46 | 6.300 | 1.113 | .427 |

Note:
NiN_50 is unitless, but the value is linearly related to intensity in dB SPL.

TABLE 3
Distribution parameters of EqL

| Frequency (Hz) | Mean | SD | Skewness | Kurtosis |
|---|---|---|---|---|
| RE 31.5 | 90.76 | 9.169 | .237 | −.547 |
| RE 50 | 85.35 | 8.732 | .871 | 1.605 |
| RE 80 | 78.12 | 8.817 | 1.337 | 1.732 |
| RE 125 | 75.53 | 7.451 | .757 | .537 |
| RE 200 | 68.82 | 6.217 | .837 | .089 |
| RE 315 | 64.29 | 4.398 | .955 | .583 |
| RE 500 | 60.82 | 2.963 | .472 | −.640 |
| RE 630 | 58.71 | 3.077 | .897 | .758 |
| RE 1250 | 57.94 | 3.092 | .465 | −.927 |
| RE 1600 | 58.00 | 4.528 | −.247 | −.237 |
| RE 2500 | 60.88 | 5.243 | .190 | .460 |
| RE 4000 | 60.71 | 6.049 | .369 | .823 |
| RE 6300 | 69.24 | 8.511 | .188 | −.967 |
| RE 8000 | 74.00 | 7.961 | −.101 | −.417 |
| RE 10000 | 73.71 | 9.674 | −.578 | .125 |
| RE 12500 | 70.18 | 12.812 | −.174 | −.587 |
| LE 31.5 | 91.24 | 10.802 | −.193 | −.534 |
| LE 50 | 80.24 | 8.519 | .842 | 2.400 |
| LE 80 | 78.06 | 8.257 | 1.032 | .410 |
| LE 125 | 73.06 | 6.878 | 1.002 | .817 |
| LE 200 | 70.59 | 9.056 | 2.791 | 9.783 |
| LE 315 | 64.29 | 4.398 | .705 | .038 |
| LE 500 | 61.76 | 4.116 | .349 | .090 |
| LE 630 | 59.82 | 5.626 | 1.649 | 2.271 |
| LE 1250 | 59.00 | 4.301 | .523 | −.526 |
| LE 1600 | 57.47 | 3.693 | −.349 | 1.196 |
| LE 2500 | 59.71 | 5.903 | .224 | 1.256 |
| LE 4000 | 59.76 | 6.505 | .460 | .601 |
| LE 6300 | 74.94 | 8.151 | .793 | .976 |
| LE 8000 | 75.00 | 7.657 | −.816 | .258 |
| LE 10000 | 77.06 | 7.327 | −1.120 | 1.102 |
| LE 12500 | 70.47 | 9.375 | −.046 | −.536 |

Note,
RE = Right Ear and LE = Left Ear.

TABLE 4
Distribution parameters of MESAS

| Frequency (Hz) | Mean | SD | Skewness | Kurtosis |
|---|---|---|---|---|
| RE 280 | .32 | .76 | −.35 | .33 |
| RE 336 | .60 | .84 | −1.59 | 3.77 |
| RE 476 | 1.08 | .80 | −1.05 | .96 |
| RE 644 | 1.38 | .82 | −1.49 | 1.76 |
| RE 868 | .79 | .52 | −2.17 | 4.22 |
| RE 1040 | −.19 | .11 | 1.16 | .78 |
| RE 1248 | −1.00 | .81 | .40 | −.48 |
| RE 1768 | −2.61 | 1.06 | .49 | −.30 |
| RE 2392 | −2.16 | 1.04 | −.67 | −.66 |
| RE 2705 | −1.31 | 1.58 | .28 | −.27 |
| RE 3224 | 1.03 | 2.07 | .86 | .87 |
| RE 3516.5 | 3.70 | 2.15 | 1.22 | 2.14 |
| RE 3922.25 | 2.84 | 2.53 | 1.11 | 2.07 |
| RE 4328 | 1.20 | 3.06 | .84 | 1.98 |
| RE 4869 | .72 | 4.09 | −.37 | 2.46 |
| LE 280 | .19 | .53 | −.50 | 1.34 |
| LE 336 | .55 | .55 | .00 | .36 |
| LE 476 | 1.05 | .54 | −.38 | .30 |
| LE 644 | 1.43 | .50 | −.47 | −1.16 |
| LE 868 | .73 | .33 | −.89 | .94 |
| LE 1040 | −.19 | .10 | .37 | −1.17 |
| LE 1248 | −1.15 | .67 | .94 | .54 |
| LE 1768 | −2.89 | .73 | −.06 | −.37 |
| LE 2392 | −2.51 | .96 | .73 | −.43 |
| LE 2705 | −1.92 | 1.28 | .94 | −.36 |
| LE 3224 | .56 | 1.51 | .36 | −.69 |
| LE 3516.5 | 3.17 | 1.32 | .19 | −1.48 |
| LE 3922.25 | 2.48 | 1.40 | .66 | −.51 |
| LE 4328 | 1.49 | 1.85 | −.06 | −.69 |
| LE 4869 | 1.48 | 2.87 | −1.05 | .99 |

Note,
RE = Right Ear and LE = Left Ear.

TABLE 5
Distribution parameters of the difference
measures based on the summary statistics

| Difference | | Frequencies | | | Skew- | |
|---|---|---|---|---|---|---|
| Measure | Ear | compared | Mean | SD | ness | Kurtosis |
| EqL | Left | Mid-Low | −16.77 | 4.11 | 0.43 | −0.61 |
| | | (dB SPL) | | | | |
| | Right | Mid-Low | −16.20 | 4.66 | −0.93 | 1.09 |
| | | (dB SPL) | | | | |
| | Left | High-Mid | 12.40 | 6.55 | −0.11 | .456 |
| | | (dB SPL) | | | | |
| MESAS | Right | High-Mid | 15.38 | 4.43 | 0.18 | −1.079 |
| | | (dB SPL) | | | | |
| | Left | Mid-Low | −0.71 | 0.97 | 0.82 | −0.13 |
| | | (dB Re: 1000 Hz) | | | | |
| | Right | Mid-Low | −0.57 | 1.69 | 1.35 | 1.79 |
| | | (dB Re: 1000 Hz) | | | | |

Note that all of the difference measures have skewness and kurtosis parameters within the range (i.e., −2 to 2) acceptable for parametric analysis.

TABLE 6
Correlation between noise tolerance and MESAS
summary statistics (right ear)

| | Low, | Mid, | Mid-Low |
|---|---|---|---|
| Right Ear | 280 to 868 Hz | 1248 to 4328 Hz | difference |
| NiN_50 | 0.047 | −0.64** | −0.54* |

**p < 0.01,
*p < 0.05.
N = 17.

TABLE 7
Correlation between noise tolerance and MESAS
summary statistics (left ear)

| | Low, | Mid, | Mid-Low |
|---|---|---|---|
| Left Ear | 280 to 868 Hz | 1248 to 4328 Hz | difference |
| NiN_50 | 0.51* | −0.22 | −0.39 |

*indicates p < 0.05.
N = 17.

TABLE 8
ANCOVA between-subjects main effect for MESAS × NiN_50

| Ear | |
|---|---|
| Left | Right |

MESAS Range(Hz)

| | 280-868 | 1248-4328 | 280-868 | 1248-4328 |
|---|---|---|---|---|
| F(1,15) | 5.36 | 0.76 | 0.034 | 10.07 |
| p | 0.035 | 0.40 | 0.86 | 0.0063 |
| ηₚ² | 0.26 | 0.048 | 0.002 | 0.40 |

Note:
The full range MESAS analysis excluded the 1040 Hz component.

TABLE 9
Correlation between left ear loudness scaling and
MESAS summary statistics

| | Low-frequency | Mid-frequency | Mid-Low |
|---|---|---|---|
| | band MESA | band MESA | MESA |
| Left Ear | (280-868 Hz) | (1248-4328 Hz) | difference |
| EqL (Mid-Low) | −0.35 | 0.77** | 0.77** |

**p < 0.01,
*p < 0.05.
N = 17.

TABLE 10
References in Example 1:

Abrams, D. A., Nicol, T., Zecker, S. G., & Kraus, N. (2006). Auditory brainstem timing
predicts cerebral asymmetry for speech. The Journal of Neuroscience, 26(43), 11131-11137.
Abrams, D. A., Nicol, T., Zecker, S., & Kraus, N. (2008). Right-hemisphere auditory
cortex is dominant for coding syllable patterns in speech. The Journal of Neuroscience,
28(15), 3958-3965.
Ahonniska, J., Cantell, M., Tolvanen, A., & Lyytinen, H. (1993). Speech perception and
brain laterality: the effect of ear advantage on auditory event-related potentials. Brain
and Language, 45(2), 127-146.
Aibara, R., Welsh, J. T., Puria, S., & Goode, R. L. (2001). Human middle-ear sound
transfer function and cochlear input impedance. Hearing Research, 152(1-2), 100-109.
Allen, J. B., Jeng, P. S., & Levitt, H. (2005). Evaluation of human middle ear function via
an acoustic power assessment. Journal of Rehabilitation Research and Development,
42(4, s2), 63-78.
Beers, A. N., Shahnaz, N., Westerberg, B. D., & Kozak, F. K. (2010). Wideband
reflectance in normal caucasian and chinese school-aged children and in children with
otitis media with effusion. Ear and Hearing, 31(2), 221-233.
Boger, M. E. E., Barbosa-Branco, A., & Ottoni, A. C. C. (2009). The noise spectrum
influence on noise-induced hearing loss prevalence in workers. Brazilian Journal of
Otorhinolaryngology, 75(3), 328-334.
Borg, E. (1972a). Acoustic middle ear reflexes: a sensory-control system. Acta Oto-
laryngologica. Supplementum, 304, 1-34.
Borg, E. (1972b). On the change in the acoustic impedance of the ear as a measure of
middle ear muscle reflex activity. Acta Oto-laryngologica, 74(3), 163-171.
Borg, E. (1973). On the neuronal organization of the acoustic middle ear reflex. A
physiological and anatomical study. Brain Research, 49(1), 101-123.
Borg, E., & Counter, S. A. (1989). The middle-ear muscles. Scientific American, 261(2),
74-80.
Borg, E., & Zakrisson, J. E. (1975). The activity of the stapedius muscle in man during
vocalization. Acta Oto-laryngologica, 79(5-6), 325-333.
Brown W. (1910). Some experimental results in the correlation of mental abilities. British
Journal of Psychology, 3, 296-322
Brown, M. C., & Levine, J. L. (2008). Dendrites of medial olivocochlear neurons in
mouse. Neuroscience, 154(1), 147-159.
Burke, K. S., Shutts, R. E., & Milo, A. P. (1967). On the Zwislocki acoustic bridge. The
Journal of the Acoustical Society of America, 41(5).
Colletti, V. (1976). Tympanomety from 200 to 2000 Hz probe tone. Audiology, 15(2),
106-119.
Colletti, V. (1977). Multifrequency tympanometry. Audiology, 16(4), 278-287.
Culling, J. F., & Darwin, C. J. (1993). Perceptual separation of simultaneous vowels:
within and across-formant grouping by f0. The Journal of the Acoustical Society of
America, 93(6), 3454-3467.
Darwin, C. (1901). The descent of man and selection in relation to sex. London: J. Murray.
Decraemer, W. F., Khanna, S. M., & Funnell, W. R. (1991). Malleus vibration mode
changes with frequency. Hearing research 54(2), 305-318.
Erickson, D. (2002). Articulation of extreme formant patterns for emphasized vowels.
Phonetica, 59(2-3), 134-149.
Feeney, M. P., & Sanford, C. A. (2004). Age effects in the human middle ear: wideband
acoustical measures. The Journal of the Acoustical Society of America, 116(6), 3546-3558.
Feeney, M. P., Keefe, D. H., & Sanford, C. A. (2004). Wideband reflectance measures
of the ipsilateral acoustic stapedius reflex threshold. Ear and Hearing, 25(5), 421-430.
Fletcher, H., & Munson, W. A. (1933). Loudness, its definition, measurement and
calculation. The Journal of the Acoustical Society of America, 5(2), 82-108.
Gordon, A. G. (1986). Abnormal middle ear muscle reflexes and audiosensitivity. British
Journal of Audiology, 20(2), 95-99.
Hawk, L. W., Stevenson, V. E., & Cook, E. W., III (1992). The effects of eyelid closure
on affective imagery and eyeblink startle. Journal of Psychophysiology, 6, 299-310.
Heffner, H. E., & Heffner, R. S. (1990). Effect of bilateral auditory cortex lesions on
absolute thresholds in japanese macaques. Journal of Neurophysiology, 64(1), 191-205.
Homma, K., Du, Y., Shimizu, Y., & Puria, S. (2009). Ossicular resonance modes of the
human middle ear for bone and air conduction. The Journal of the Acoustical Society of
America, 125(2), 968-979.
Honjo, I., Ushiro, K., Haji, T., Nozoe, T., & Matsui, H. (1983). Role of the tensor tympani
muscle in eustachian tube function. Acta Otolaryngol, 95(1-4), 329-332.
Hornickel, J., Skoe, E., & Kraus, N. (2009). Subcortical laterality of speech encoding.
Audiology and Neurotology, 14(3), 198-207.
Hugdahl, K., Carlsson, G., & Eichele, T. (2001). Age effects in dichotic listening to
consonant-vowel syllables: interactions with attention. Developmental Neuropsychology,
20(1), 445-457.
Hugdahl, K., Carlsson, G., & Eichele, T. (2001). Age effects in dichotic listening to
consonant-vowel syllables: interactions with attention. Developmental Neuropsychology,
20(1), 445-457.
Hugdahl, K., Westerhausen, R., Alho, K., Medvedev, S., & Hämäläinen, H. (2008). The
effect of stimulus intensity on the right ear advantage in dichotic listening. Neuroscience
Letters, 431(1), 90-94.
Hunter, L. L., Feeney, M. P., Lapsley Miller, J. A., Jeng, P. S., & Bohning, S. (2010).
Wideband reflectance in newborns: Normative regions and relationship to hearing-
screening results. Ear and Hearing, 31(5), 599-610.
Irvine, D. R. (1976). Effects of reflex middle-ear muscle contractions on cochlear
responses to bone-conducted sound. Audiology, 15(5), 433-444.
Katzenell, U., & Segal, S. (2001). Hyperacusis: review and clinical guidelines. Otology &
Neurotology, 22(3).
Keefe, D. H., Bulen, J. C., Arehart, K. H., & Burns, E. M. (1993). Ear-canal impedance
and reflection coefficient in human infants and adults. The Journal of the Acoustical
Society of America, 94(5), 2617-2638.
Khalfa, S., Bruneau, N., Roge, B., Georgieff, N., Veuillet, E., Adrien, J., Barthelemy, C.,
& Collet, L. (2004). Increased perception of loudness in autism. Hearing Research,
198(1-2), 87-92.
Khalfa, S., Dubal, S., Veuillet, E., Perez-Diaz, F., Jouvent, R., & Collet, L. (2002).
Psychometric normalization of a hyperacusis questionnaire. ORL; Journal for Oto-rhino-
laryngology and its Related Specialties, 64(6), 436-442.
King, C., Nicol, T., McGee, T., & Kraus, N. (1999). Thalamic asymmetry is related to
acoustic signal complexity. Neuroscience Letters, 267(2), 89-92.
Kofler, M., Muller, J., Rinnerthaler-Weichbold, M., & Valls-Solé, J. (2008). Laterality of
auditory startle responses in humans. Clinical Neurophysiology, 119(2), 309-314.
Koike, T., Wada, H., & Kobayashi, T. (2002). Modeling of the human middle ear using
the finite-element method. The Journal of the Acoustical Society of America, 111(3),
1306-1317.
Kryter, K. D. (1985). The effects of noise on man. New York: Academic Press.
Lange, N., Dubray, M. B., Lee, J. E. E., Froimowitz, M. P., Froehlich, A., Adluru, N.,
Wright, B., Ravichandran, C., Fletcher, P. T., Bigler, E. D., Alexander, A. L., & Lainhart, J. E.
(2010). Atypical diffusion tensor hemispheric asymmetry in autism. Autism
Research, 3(6), 350-358.
Levine, R. A., Liederman, J., & Riley, P. (1988). The brainstem auditory evoked potential
asymmetry is replicable and reliable. Neuropsychologia, 26(4), 603-614.
Liberman, M. C., & Guinan, J. J. (1998). Feedback control of the auditory periphery:
anti-masking effects of middle ear muscles vs. olivocochlear efferents. Journal of
Communication Disorders, 31(6).
Lutman, M. E. (1980). Real-ear calibration of ipsilateral acoustic reflex stimuli from five
types of impedance meter. Scandinavian Audiology, 9(3), 137-145.
Lutman, M., & Martin, A. (1979). Development of an electroacoustic analogue model of
the middle ear and acoustic reflex. Journal of Sound and Vibration, 64(1), 133-157.
Margolis, R. H., & Goycoolea, H. G. (1993). Multifrequency tympanometry in normal
adults. Ear and Hearing, 14(6), 408-413.
Margolis, R. H., Hunter, L. L., & Giebink, G. S. (1994). Tympanometric evaluation of
middle ear function in children with otitis media. The Annals of Otology, Rhinology &
Laryngology. Supplement, 163, 34-38.
Margolis, R. H., Van Camp, K. J., Wilson, R. H., & Creten, W. L. (1985). Multifrequency
tympanometry in normal ears. Audiology, 24(1), 44-53.
Mukerji, S., Windsor, A. M. M., & Lee, D. J. (2010). Auditory brainstem circuits that
mediate the middle ear muscle reflex. Trends in Amplification, 14(3), 170-191.
Nageris, B. I., Raveh, E., Zilberberg, M., & Attias, J. (2007). Asymmetry in noise-induced
hearing loss: relevance of acoustic reflex and left or right handedness. Otology &
Neurotology, 28(4), 434-437.
Pang, X. D. (1989). Effects of stapedius-muscle contractions on masking of tone
responses in the auditory nerve. Research Laboratory of Electronics, Massachusetts
Institute of Technology.
Pang, X. D., & Guinan, J. J. (1997). Effects of stapedius-muscle contractions on the
masking of auditory-nerve responses. The Journal of the Acoustical Society of America,
102(6), 3576-3586.
Perry, W., Minassian, A., Lopez, B., Maron, L., & Lincoln, A. (2007). Sensorimotor
gating deficits in adults with autism. Biological Psychiatry, 61(4), 482-486.
Pirila, T. (1991a). Left-right asymmetry in the human response to experimental noise
exposure. I. Interaural correlation of the temporary threshold shift at 4 kHz frequency.
Acta oto-laryngologica, 111(4), 677-683.
Pirila, T. (1991b). Left-right asymmetry in the human response to experimental noise
exposure. II. Pre-exposure hearing threshold and temporary threshold shift at 4 kHz
frequency. Acta oto-laryngologica, 111(5), 861-866.
Porges, S. W. (2007). The polyvagal perspective. Biological psychology, 74(2), 116-143.
Porges, SW and Lewis, GF (2010). The polyvagal hypothesis: common mechanisms
mediating autonomic regulation, vocalizations and listening. In, Handbook of
mammalian vocalization: An integrative neuroscience approach. Ed: Brudzynski, S.
Amsterdam: Elsevier/Academic Press.
Porges, SW, JA Doussard-Roosevelt, Maiti, AK (1994). Vagal tone and the
physiological regulation of emotion. In The Development of Emotion Regulation:
Biological and Behavioral Considerations, N.A. Fox, Ed. Monographs of the Society for
Reseach in Child Development, 59(2-3, Serial No. 240), 167-188.
Rhodes, M., Margolis, R., Hirsch, J., & Napp, A. (1999). Hearing screening in the
newborn intensive care nursery: Comparison of methods. Otolaryngology - Head and
Neck Surgery, 120(6), 799-808.
Roberts, B., Summers, R. J., & Bailey, P. J. (2010). The intelligibility of noise-vocoded
speech: spectral information available from across-channel comparison of amplitude
envelopes. Proceedings. Biological sciences/The Royal Society.
Rouiller, E. M., Capt, M., Dolivo, M., & De Ribaupierre, F. (1989). Neuronal organization
of the stapedius reflex pathways in the rat: a retrograde HRP and viral transneuronal
tracing study. Brain Research, 476(1), 21-28.
Rowe, T. (1996). Coevolution of the mammalian middle ear and neocortex. Science,
273(5275), 651-654.
Russo, N., Nicol, T., Trommer, B., Zecker, S., & Kraus, N. (2009a). Brainstem
transcription of speech is disrupted in children with autism spectrum disorders.
Developmental Science, 12(4), 557-567.
Russo, N., Zecker, S., Trommer, B., Chen, J., & Kraus, N. (2009b). Effects of
background noise on cortical encoding of speech in autism spectrum disorders. Journal
of Autism and Developmental Disorders, 39(8), 1185-1196.
Schutte, M., Marks, A., Wenning, E., & Griefahn, B. (2007). The development of the
noise sensitivity questionnaire. Noise and Health, 9(34), 15+.
Shahnaz, N., & Polka, L. (1997). Standard and multifrequency tympanometry in normal
and otosclerotic ears. Ear and Hearing, 18(4), 326-341.
Shrout, P. E. (1998). Measurement reliability and agreement in psychiatry. Statistical
Methods in Medical Research, 7(3), 301-317.
Simmons, F. B., & Beatty, D. L. (1962). A theory of middle ear muscle function at
moderate sound levels. Science, 138(3540), 590-592.
Sininger, Y. S., & Cone-Wesson, B. (2004). Asymmetric cochlear processing mimics
hemispheric specialization. Science (New York, N.Y.), 305(5690).
Sininger, Y. S., & Cone-Wesson, B. (2006). Lateral asymmetry in the ABR of neonates:
evidence and mechanisms. Hearing Research, 212(1-2), 203-211.
Sininger, Y., & Cone, B. (2008). Comment on “ear asymmetries in middle-ear, cochlear,
and brainstem responses in human infants”. The Journal of the Acoustical Society of
America, 124(3), 1401-1403.
Spearman C. (1910). Correlation calculated from faulty data. British Journal of
Psychology, 3, 271-95.
Stansfeld, S. A. (1992). Noise, noise sensitivity and psychiatric disorder: epidemiological
and psychophysiological studies. Psychological Medicine, Supplement 22, 1-44.
Stenfelt, S. (2006). Middle ear ossicles motion at hearing thresholds with air conduction
and bone conduction stimulation. The Journal of the Acoustical Society of America,
119(5, p1), 2848-2858.
Summers, R. J., Bailey, P. J., & Roberts, B. (2010). Effects of differences in
fundamental frequency on across-formant grouping in speech perception. The Journal
of the Acoustical Society of America, 128(6), 3667-3677.
Suzuki, Y., & Takeshima, H. (2004). Equal-loudness-level contours for pure tones. The
Journal of the Acoustical Society of America, 116(2), 918-933.
Tartter, V. C., & Braun, D. (1994). Hearing smiles and frowns in normal and whisper
registers. The Journal of the Acoustical Society of America, 96(4), 2101-2107.
Vallejo, L. A. A., Hidalgo, A., Lobo, F., Tesorero, M. A. A., Gil-Carcedo, E., Sánchez, E.,
& Gil-Carcedo, L. M. (2010). Is the middle ear the first filter of frequency selectivity?
Acta Otorrinolaringologica Espa\tilde{n}ola, 61(2), 118-127.
Voyer, D., & Ingram, J. (2005). Attention, reliability, and validity of perceptual
asymmetries in the fused dichotic words test. Laterality: Asymmetries of Body, Brain and
Cognition, 10(6), 545-561.
Wang, Y., Hu, Y., Meng, J., & Li, C. (2001). An ossified meckel's cartilage in two
cretaceous mammals and origin of the mammalian middle ear. Science (New York,
N.Y.), 294(5541), 357-361.
Watson, L. R., Baranek, G. T., Roberts, J. E., David, F. J., & Perryman, T. Y. (2010).
Behavioral and physiological responses to child-directed speech as predictors of
communication outcomes in children with autism spectrum disorders. Journal of
Speech, Language, and Hearing Research, 53(4), 1052.
Willi, U. B., Ferrazzini, M. A., & Huber, A. M. (2002). The incudo-malleolar joint and
sound transmission losses. Hearing Research, 174(1-2), 32-44.
Zwislocki, J. J. (2002). Auditory system: Peripheral nonlinearity and central additivity, as
revealed in the human stapedius-muscle reflex. Proceedings of the National Academy of
Sciences of the United States of America, 99(22), 14601-14606.

1. A method of evaluating dynamic middle ear muscle activity in a subject having ossicles, said method comprising the steps of:
introducing a non-harmonic acoustic input to an ear of the subject, wherein said non-harmonic acoustic input comprises a comb input that includes frequencies in each of a low frequency range, a middle frequency range and high frequency range, wherein the three frequency ranges span an input frequency range that is at least greater than or equal to 100 Hz and less than or equal to 10,000 Hz, wherein the ear has an intact ossicle chain having ossicles capable of movement in ossicle directions, and said non-harmonic acoustic input generates movement of the ossicles in all available ossicle directions; and
measuring reflected energy from the ear during said non-harmonic acoustic input that generates movement of the ossicles in all available direction, thereby evaluating dynamic middle ear muscle activity.

2. The method of claim 1, wherein:
the low frequency range is less than or equal to approximately 1000 Hz;
the middle frequency range is greater than approximately 1000 Hz and less than approximately 3000 Hz; and
the high frequency range is greater than or equal to approximately 3000 Hz.

3. The method of claim 1, wherein the measuring step has a measuring time period and the non-harmonic acoustic input is continuously introduced to the ear during the measuring time period.

4. The method of claim 1, wherein the non-harmonic acoustic input is continuously introduced to the ear for a time that is greater than or equal to 0.5 second.

5. The method of claim 1, wherein the measuring comprises:
measuring said reflected energy over a measuring frequency range and obtaining dynamic middle ear muscle activity as a function frequency.

6. The method of claim 5, wherein the measuring frequency range is selected from a range that is greater than or equal to 200 Hz and less than or equal to 5000 Hz.

7. The method of claim 1, wherein said evaluating is by obtaining a magnitude of the reflected energy at a measured frequency.

8. The method of claim 7, further comprising comparing the obtained magnitude against a reference from a normal subject.

9. The method of claim 8, wherein the comparing is for the magnitude of the reflected energy over a range of measured frequency.

10. The method of claim 9, further comprising calculating a difference between the obtained magnitude and the reference magnitude at a one or more measured frequency that is within the range of measured frequency.

11. The method of claim 10, further comprising calculating a composite measure by weighting at a one or more weighted frequency value.

12. The method of claim 11, wherein the weighted frequency value corresponds to a frequency associated with an atypical hearing condition or a sound processing defect.

13. The method of claim 12, wherein the atypical hearing defect is:
difficulty in hearing speech in a noisy environment and the weighted frequency value is selected from a frequency that is greater than 1300 HZ;
hypersensitivity to speech and the weighted frequency value is selected from a frequency that is between about 1300 Hz and 4000 Hz;
hearing loss and the weighted frequency value is selected from a frequency that is between about 1000 Hz and 5000 Hz;
hypersensitivity to noise and the weighted frequency value is between about 50 Hz and 1000 Hz; or
impaired language development and the weighted frequency value is greater than 1300 Hz.

14. The method of claim 1, wherein said comb input comprises a plurality of components each having a non-harmonic frequency, said components spanning a frequency range that is greater than or equal to about 50 Hz and less than or equal to about 15000 Hz.

15. The method of claim 14, wherein at least two components are provided in each of the low, middle and high frequency ranges.

16. The method of claim 14, wherein said components have a total number selected from a range that is greater than or equal to 3 and less than or equal to 100.

17. (canceled)

18. The method of claim 14, wherein said comb input comprises components that are not integer harmonics.

19. The method of claim 14, wherein each of said components have substantially equivalent power levels to the other components, and said power levels remain substantially constant during said introducing step.

20. The method of claim 1, further comprising selecting the comb input to minimize or avoid generating standing waves of air pressure on the reflected energy.

21. The method of claim 14, wherein each of said components is a non-square wave having a full-width at half-maximum that is less than or equal to 5 Hz.

22. The method of claim 5, wherein said evaluating comprises determining the difference between the measured reflected energy and a normal reflected energy from a normal subject.

23. The method of claim 1, wherein said middle ear muscle activity is identified as atypical.

24. The method of claim 1, further comprising obtaining information useful for diagnosing a middle-ear related abnormality, wherein said abnormality is selected from the group consisting of: conductive hearing loss; auditory processing deficits; noise hypersensitivity; speech hypersensitivity and speech hyposensitivity.

25. The method of claim 24, wherein said information corresponds to higher reflected energy at a higher frequency, wherein said higher frequency is greater than or equal to 2000 Hz.

26. The method of claim 1, further comprising quantifying dynamic middle ear muscle activity for a subject suspected of a clinical disorder or under a therapeutic treatment of a clinical disorder.

27. The method of claim 26, wherein the clinical disorder is selected from the group consisting of autism, post-traumatic stress disorder, language delay, language disorder, and hearing disorder.

28. The method of claim 1, further comprising presenting a middle ear muscle acoustic challenge to an ear contralateral to the ear in sound-wave communication with the non-harmonic acoustic input.

29. The method of claim 23, further comprising providing the subject with a therapeutic intervention and monitoring the effectiveness of the therapeutic intervention by repeating the evaluation of dynamic middle ear activity after the therapeutic intervention.

30. The method of claim 1, further comprising introducing a probe tone to the ear at a frequency and intensity selected to elicit an acoustic response contraction of the middle ear muscles.

31. A method of measuring a resting tension of middle ear muscles in a subject having an intact ossicle chain, said method comprising the steps of:
exciting each ossicle of said ossicle chain by introducing a non-harmonic acoustic input to an ear of the subject, thereby causing each of the ossicles to move in all available ossicle movement directions; and
measuring reflected energy from the ear during said non-harmonic acoustic input that generates movement of the ossicles in all available directions, thereby measuring the resting tension of middle ear muscles.

32. The method of claim 31, wherein the measured resting tension of the middle ear muscle provides information useful in diagnosing a hearing or psychiatric condition.

33. The method of claim 1, wherein a high-reliability status of the middle ears of both ears of the subject is assessed in an assessment time that is less than or equal to five minutes.

34. The method of claim 1, wherein the comb input is provided at an intensity that is insufficient to generate an acoustic reflex response in the subject.

35. A device for measuring a resting tension of middle ear muscles in an active ear of a subject, said device comprising:
a. a signal generator for generating a steady-state non-harmonic acoustic input comprising a comb input;
b. a speaker for emitting a sound wave that is generated from the signal generator;
c. a probe containing the speaker for positioning the speaker in sound-communication with an ear, wherein the emitted sound wave vibrates ossicles of an intact ossicle chain of the ear in all available ossicle directions;
d. a microphone in sound wave communication with the speaker for detecting a reflected sound wave of the emitted sound wave during ossicle vibration in all available ossicle directions; and
e. a processor for calculating changes in an acoustic transfer function from middle ear muscle movement based on a reflectance phase shift or magnitude change between the emitted sound wave and the reflected sound wave, wherein the emitted sound wave, detected reflected sound wave, and calculated acoustic transfer function are continuous and synchronized with the emitted sound wave.

36. The device of claim 35, wherein the acoustic transfer function is calculated by spectral analysis with frequency dependent resolution having a tolerance for each component of the comb signal within 0.1 radians per second, thereby minimizing effects of bodily noise.

37. The device of claim 35, wherein the comb input comprises a plurality of components each having a non-harmonic frequency, said components spanning a frequency range that is greater than or equal to about 50 Hz and less than or equal to about 15000 Hz, and at least one component is in each of a low frequency range that is less than or equal to about 1000 Hz, a middle frequency range greater than approximately 1000 Hz and less than approximately 3000 Hz; and high frequency range greater than or equal to approximately 3000 Hz.
